SEQUENCE LISTING (53 sequences)

SEQ ID NO: 1 - protein, 454 aa, Homo sapiens
  MISC_FEATURE: Human B7-H6
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 2 - protein, 121 aa, Artificial Sequence
  DeltaB7-H6 wt
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPAS

SEQ ID NO: 3 - protein, 121 aa, Artificial Sequence
  DeltaB7-H6 sequence with modification sites X1-X8
  SITE (36)..(36): Any amino acid
  SITE (38)..(38): Any amino acid
  SITE (58)..(58): Any amino acid
  SITE (59)..(59): Any amino acid
  SITE (101)..(101): Any amino acid
  SITE (103)..(103): Any amino acid
  SITE (105)..(105): Any amino acid
  SITE (106)..(106): Any amino acid
DLKVEMMAGGTQITPLNDNVTIFCNIFYSQPLNITXMXITWFWKSLTFDKEVKVFEFXXDHQEAFRPGAIVSPWRLKSGDASLRLPGIQLEEAGEYRCEVXVXPXXAQGTVQLEVVASPAS

SEQ ID NO: 4 - protein, 121 aa, Artificial Sequence
  DeltaB7-H6_S3#1
DLKVEMMAGGTQITPLNDNVTIFCNIFYSQPLNITTMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 5 - protein, 121 aa, Artificial Sequence
  DeltaB7-H6_S3#2
DLKVEMMAGGTQITPLNDNVTIFCNIFYSQPLNITWMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 6 - protein, 121 aa, Artificial Sequence
  DeltaB7-H6_S3#3
DLKVEMMAGGTQITPLNDNVTIFCNIFYSQPLNITHMGITWFWKSLTFDKEVKVFEFFGDHQEAFRPGAIVSPWRLKSGDASLRLPGIQLEEAGEYRCEVFVTPYKAQGTVQLEVVASPAS

SEQ ID NO: 7 - protein, 121 aa, Artificial Sequence
  DeltaB7-H6_S3#4
DLKVEMMAGGTQITPLNDNVTIFCNIFYSQPLNITIMGITWFWKSLTFDKEVKVFEFWGDHQEAFRPGAIVSPWRLKSGDASLRLPGIQLEEAGEYRCEVVVTPLKAQGTVQLEVVASPAS

SEQ ID NO: 8 - protein, 121 aa, Artificial Sequence
  DeltaB7-H6_S3#5
DLKVEMMAGGTQITPLNDNVTIFCNIFYSQPLNITVMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 9 - protein, 121 aa, Artificial Sequence
  DeltaB7-H6_S3#6
DLKVEMMAGGTQITPLNDNVTIFCNIFYSQPLNITHMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 10 - protein, 121 aa, Artificial Sequence
  DeltaB7-H6_S3#7
DLKVEMMAGGTQITPLNDNVTIFCNIFYSQPLNITHMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 11 - protein, 121 aa, Artificial Sequence
  DeltaB7-H6_S3#8
DLKVEMMAGGTQITPLNDNVTIFCNIFYSQPLNITHMGITWFWKSLTFDKEVKVFEFWGDHQEAFRPGAIVSPWRLKSGDASLRLPGIQLEEAGEYRCEVIVTPLKAQGTVQLEVVASPAS

SEQ ID NO: 12 - protein, 121 aa, Artificial Sequence
  DeltaB7-H6_S3#9
DLKVEMMAGGTQITPLNDNVTIFCNIFYSQPLNITHMGITWFWKSLTFDKEVKVFEFFGDHQEAFRPGAIVSPWRLKSGDASLRLPGIQLEEAGEYRCEVVVTPYKAQGTVQLEVVASPAS

SEQ ID NO: 13 - protein, 121 aa, Artificial Sequence
  DeltaB7-H6_S3#10
DLKVEMMAGGTQITPLNDNVTIFCNIFYSQPLNITSMGITWFWKSLTFDKEVKVFEFYGDHQEAFRPGAIVSPWRLKSGDASLRLPGIQLEEAGEYRCEVFVTPYKAQGTVQLEVVASPAS

SEQ ID NO: 14 - protein, 121 aa, Artificial Sequence
  DeltaB7-H6_S3#11
DLKVEMMAGGTQITPLNDNVTIFCNIFYSQPLNITLMGITWFWKSLTFDKEVKVFEFWGDHQEAFRPGAIVSPWRLKSGDASLRLPGIQLEEAGEYRCEVIVTPFKAQGTVQLEVVASPAS

SEQ ID NO: 15 - protein, 121 aa, Artificial Sequence
  DeltaB7-H6_S3#12
DLKVEMMAGGTQITPLNDNVTIFCNIFYSQPLNITEMGITWFWKSLTFDKEVKVFEFFGDHQEAFRPGAIVSPWRLKSGDASLRLPGIQLEEAGEYRCEVVVTPYKAQGTVQLEVVASPAS

SEQ ID NO: 16 - protein, 121 aa, Artificial Sequence
  DeltaB7-H6_S3#13
DLKVEMMAGGTQITPLNDNVTIFCNIFYSQPLNITSMIITWFWKSLTFDKEVKVFEFYGDHQEAFRPGAIVSPWRLKSGDASLRLPGIQLEEAGEYRCEVVVTPLKAQGTVQLEVVASPAS

SEQ ID NO: 17 - protein, 121 aa, Artificial Sequence
  DeltaB7-H6_S3#14
DLKVEMMAGGTQITPLNDNVTIFCNIFYSQPLNITTMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 18 - protein, 121 aa, Artificial Sequence
  DeltaB7-H6_S3#15
DLKVEMMAGGTQITPLNDNVTIFCNIFYSQPLNITIMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 19 - protein, 121 aa, Artificial Sequence
  DeltaB7-H6_S3#16
DLKVEMMAGGTQITPLNDNVTIFCNIFYSQPLNITLMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 20 - protein, 121 aa, Artificial Sequence
  DeltaB7-H6_S3#17
DLKVEMMAGGTQITPLNDNVTIFCNIFYSQPLNITSMGITWFWKSLTFDKEVKVFEFYGDHQEAFRPGAIVSPWRLKSGDASLRLPGIQLEEAGEYRCEVFVTPLKAQGTVQLEVVASPAS

SEQ ID NO: 21 - protein, 121 aa, Artificial Sequence
  DeltaB7-H6_S3#18
DLKVEMMAGGTQITPLNDNVTIFCNIFYSQPLNITYMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 22 - protein, 121 aa, Artificial Sequence
  DeltaB7-H6_S3#19
DLKVEMMAGGTQITPLNDNVTIFCNIFYSQPLNITQMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 23 - protein, 121 aa, Artificial Sequence
  DeltaB7-H6_S3#20
DLKVEMMAGGTQITPLNDNVTIFCNIFYSQPLNITHMGITWFWKSLTFDKEVKVFEFWGDHQEAFRPGAIVSPWRLKSGDASLRLPGIQLEEAGEYRCEVFVTPLKAQGTVQLEVVASPAS

SEQ ID NO: 24 - protein, 121 aa, Artificial Sequence
  DeltaB7-H6_S3#21
DLKVEMMAGGTQITPLNDNVTIFCNIFYSQPLNITLMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 25 - protein, 121 aa, Artificial Sequence
  DeltaB7-H6_S3#22
DLKVEMMAGGTQITPLNDNVTIFCNIFYSQPLNITEMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 26 - protein, 121 aa, Artificial Sequence
  DeltaB7-H6_S3#23
DLKVEMMAGGTQITPLNDNVTIFCNIFYSQPLNITEMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 27 - protein, 121 aa, Artificial Sequence
  DeltaB7-H6_S3#24
DLKVEMMAGGTQITPLNDNVTIFCNIFYSQPLNITIMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 28 - protein, 121 aa, Artificial Sequence
  DeltaB7-H6_S3#25
DLKVEMMAGGTQITPLNDNVTIFCNIFYSQPLNITWMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 29 - protein, 121 aa, Artificial Sequence
  DeltaB7-H6_S3#26
DLKVEMMAGGTQITPLNDNVTIFCNIFYSQPLNITTMGITWFWKSLTFDKEVKVFEFWDDHQEAFRPGAIVSPWRLKSGDASLRLPGIQLEEAGEYRCEVVVTPYKAQGTVQLEVVASPAS

SEQ ID NO: 30 - protein, 121 aa, Artificial Sequence
  DeltaB7-H6_S3#27
DLKVEMMAGGTQITPLNDNVTIFCNIFYSQPLNITTMGITWFWKSLTFDKEVKVFEFWGDHQEAFRPGAIVSPWRLKSGDASLRLPGIQLEEAGEYRCEVIVTPYKAQGTVQLEVVASPAS

SEQ ID NO: 31 - protein, 121 aa, Artificial Sequence
  DeltaB7-H6_S3#28
DLKVEMMAGGTQITPLNDNVTIFCNIFYSQPLNITHMGITWFWKSLTFDKEVKVFEFWGDHQEAFRPGAIVSPWRLKSGDASLRLPGIQLEEAGEYRCEVVVTPLKAQGTVQLEVVASPAS

SEQ ID NO: 32 - protein, 121 aa, Artificial Sequence
  DeltaB7-H6_S3#29
DLKVEMMAGGTQITPLNDNVTIFCNIFYSQPLNITVMGITWFWKSLTFDKEVKVFEFWGDHQEAFRPGAIVSPWRLKSGDASLRLPGIQLEEAGEYRCEVIVTPYKAQGTVQLEVVASPAS

SEQ ID NO: 33 - protein, 121 aa, Artificial Sequence
  DeltaB7-H6_S3#30
DLKVEMMAGGTQITPLNDNVTIFCNIFYSQPLNITEMGITWFWKSLTFDKEVKVFEFWGDHQEAFRPGAIVSPWRLKSGDASLRLPGIQLEEAGEYRCEVIVTPLKAQGTVQLEVVASPAS

SEQ ID NO: 34 - protein, 121 aa, Artificial Sequence
  DeltaB7-H6_S3#31
DLKVEMMAGGTQITPLNDNVTIFCNIFYSQPLNITIMGITWFWKSLTFDKEVKVFEFYGDHQEAFRPGAIVSPWRLKSGDASLRLPGIQLEEAGEYRCEVVVTPLKAQGTVQLEVVASPAS

SEQ ID NO: 35 - protein, 121 aa, Artificial Sequence
  DeltaB7-H6_S3#3
DLKVEMMAGGTQITPLNDNVTIFCNIFYSQPLNITEMGITWFWKSLTFDKEVKVFEFWGDHQEAFRPGAIVSPWRLKSGDASLRLPGIQLEEAGEYRCEVVVTPVKAQGTVQLEVVASPAS

SEQ ID NO: 36 - protein, 121 aa, Artificial Sequence
  DeltaB7-H6_S2#1
DLKVEMMAGGTQITPLNDNVTIFCNIFYSQPLNITSMGITWFWKSLTFDKEVKVFEFYGDHQEAFRPGAIVSPWRLKSGDASLRLPGIQLEEAGEYRCEVVVTPYKAQGTVQLEVVASPAS

SEQ ID NO: 37 - protein, 121 aa, Artificial Sequence
  DeltaB7-H6_S2#2
DLKVEMMAGGTQITPLNDNVTIFCNIFYSQPLNITSMGITWFWKSLTFDKEVKVFEFWDDHQEAFRPGAIVSPWRLKSGDASLRLPGIQLEEAGEYRCEVVVTPYKAQGTVQLEVVASPAS

SEQ ID NO: 38 - protein, 121 aa, Artificial Sequence
  DeltaB7-H6_S2#3
DLKVEMMAGGTQITPLNDNVTIFCNIFYSQPLNITTMGITWFWKSLTFDKEVKVFEFWGDHQEAFRPGAIVSPWRLKSGDASLRLPGIQLEEAGEYRCEVVVTPLKAQGTVQLEVVASPAS

SEQ ID NO: 39 - protein, 121 aa, Artificial Sequence
  DeltaB7-H6_S2#4
DLKVEMMAGGTQITPLNDNVTIFCNIFYSQPLNITHMGITWFWKSLTFDKEVKVFEFYADHQEAIRPGAIVSPWRLKSGDASLRLPGIQLEEAGEYRCEVVVTPYKAQGTVQLEVVASPAS

SEQ ID NO: 40 - protein, 121 aa, Artificial Sequence
  DeltaB7-H6_S2#5
DLKVEMMAGGTQITPLNDNVTIFCNIFYSQPLNITSMGITWLWKSLTFDKEVKVFEFWGDHQEAFRPGAIVSPWRLKSGDASLRLPGIQLEEAGEYRCEVFVTPYKAQGTVQLEVVASPAS

SEQ ID NO: 41 - protein, 121 aa, Artificial Sequence
  DeltaB7-H6_S2#6
DLKVEMMAGGTQITPLNDNVTIFCNIFYFQPLNITEMGITWFWKSLTFDKEVKVFEFFGDHQEAFRPGAIVSPWRLKSGDASLRLPGIQLEEAGEYRCEVIVTPLKAQGTVQLEVVASPAS

SEQ ID NO: 42 - protein, 121 aa, Artificial Sequence
  DeltaB7-H6_S2#7
DLKVEMMAGGTQITPLNDNVTIFCNIFYSQPLNITSMGITWFWKSLTFDKEVKVFEFYGDHQEAFRPGAIVSPWRLKSGDASLRLPGIQLEEAGEYRCEVIVTPYKAQGTVQLEVVTSPAS

SEQ ID NO: 43 - protein, 121 aa, Artificial Sequence
  DeltaB7-H6_S2#8
DLKVEMMAGGTQITPLNDNVTIFCNIFYSQPLNITTMGITWFWKSLTFDKEVKVFEFWGDHQEAFRPGAIVSPWRLKSGDASLRLPGIQLEEAGEYRCGVVVTPVKAQGTVQLEVVASPAS

SEQ ID NO: 44 - protein, 121 aa, Artificial Sequence
  DeltaB7-H6_S2#9
DLKVEMMAGGTQITPLNDNVTIFCNIFYSQPLNITSMGITWFWKSLTFDKEVKVFEFYGDHQEAFRPGAIVSPWRLKSGDASLRLPGIQLEEAGEYRCEVVVTPLKAQGTAQLEVVASPAS

SEQ ID NO: 45 - protein, 121 aa, Artificial Sequence
  DeltaB7-H6_S2#10
DLKVEMMAGGTQITPLNDNVTIFCNIFYSQPLNITLMGITWFWKSLTFDKEVKVFEFWGDHQEAFRPGAIVSPWGLKSGDASLRLPGIQLEEAGEYRCEVVVTPLKAQGTVQLEVVASPAS

SEQ ID NO: 46 - protein, 121 aa, Artificial Sequence
  DeltaB7-H6_S2#11
DLKVEMMAGGTQITPLNDNVTIFCNIFYSQPLNITTMGITWFWKSLTFDKEVKVFEFWQDHQEAFRPGAIVSPWRLKSGDASLRLPGIQLEEAGEYRCEVVVTPLKAQGTVQLEVVASPAS

SEQ ID NO: 47 - protein, 121 aa, Artificial Sequence
  DeltaB7-H6_S2#12
DLKVEMMAGGTQITPLNDNVTIFCNIFYSQPLNITHMGITWLWKSLTFDKEVKVFEFYGDHQEAFRPGAIVSPWRLKSGDASLRLPGIQLEEAGEYRCEVVVTPLKAQGTVQLEVVASPAS

SEQ ID NO: 48 - protein, 121 aa, Artificial Sequence
  DeltaB7-H6_S2#13
DLKVEMMAGGTQITPLNDNVTIFCNIFYSQPLNITSMGITWFWKSLTFDKEVKVFEFWGDHQEAFRPGAIVSPWRLKSGDASLRLPGIQLEEAGEYRCEVVVTPYKAQGTVQLEVVASPAS

SEQ ID NO: 49 - protein, 121 aa, Artificial Sequence
  DeltaB7-H6_S2#14
DLKVEMMAGGTQITPLNDNVTIFCNIFYSQPLNITTMGITWFWKSLTFDKEVKVFEFFGDHQEAFRPGAIVSPWRLKSGDASLRLPGIQLEEAGEYRCEVVVTPYKAQGTVQLEVVASPAS

SEQ ID NO: 50 - protein, 121 aa, Artificial Sequence
  DeltaB7-H6_S2#15
  SITE (66)..(66): Any amino acid
  SITE (84)..(84): Any amino acid
DLKVEMMAGGTQITPLNDNVTIFCNIFYSQPLNITEMGITWFWKSLTFDKEVKVFEFYGDHQEAFXPGAIVSPWRLKSGDASLXLPGIQLEEAGEYRCEVVVTPLKAQGTVQLEVVASPAS

SEQ ID NO: 51 - protein, 121 aa, Artificial Sequence
  DeltaB7-H6 sequence with modification sites X1, X3, X4, X5 and X7
  SITE (36)..(36): Any amino acid
  SITE (58)..(58): Any amino acid
  SITE (59)..(59): Any amino acid
  SITE (101)..(101): Any amino acid
  SITE (105)..(105): Any amino acid
DLKVEMMAGGTQITPLNDNVTIFCNIFYSQPLNITXMGITWFWKSLTFDKEVKVFEFXXDHQEAFRPGAIVSPWRLKSGDASLRLPGIQLEEAGEYRCEVXVTPXKAQGTVQLEVVASPAS

SEQ ID NO: 52 - protein, 121 aa, Artificial Sequence
  DeltaB7-H6 sequence with modification sites X6 and X8
  SITE (103)..(103): Any amino acid
  SITE (106)..(106): Any amino acid
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVXPLXAQGTVQLEVVASPAS

SEQ ID NO: 53 - protein, 121 aa, Artificial Sequence
  DeltaB7-H6 sequence with modification sites X2, X6 and X8
  SITE (38)..(38)
  SITE (103)..(103)
  SITE (106)..(106)
DLKVEMMAGGTQITPLNDNVTIFCNIFYSQPLNITSMXITWFWKSLTFDKEVKVFEFFGDHQEAFRPGAIVSPWRLKSGDASLRLPGIQLEEAGEYRCEVVVXPLXAQGTVQLEVVASPAS

(19) United States
(12) Patent Application Publication
(10) Pub. No.: US 2023/0287082 A1
(43) Pub. Date: Sep. 14, 2023

(54) NKP30 BINDERS

(71) Applicant: Merck Patent GmbH, Darmstadt (DE)

(72) Inventors: Stefan Zielonka, Darmstadt (DE); Lars Toleikis, Darmstadt (DE); Simon Krah, Darmstadt (DE); Lukas Pekar, Darmstadt (DE)

(73) Assignee: Merck Patent GmbH, Darmstadt (DE)

(21) Appl. No.: 18/024,057

(22) PCT Filed: Aug. 31, 2021
(86) PCT No.: PCT/EP2021/073990

§ 371 Date: Mar. 1, 2023

(30) Foreign Application Priority Data
Sep. 1, 2020 (EP) 20193930.3
Oct. 14, 2020 (EP) 20201860.2

(51) Int. Cl. C07K 14/705

(52) U.S. Cl. CPC C07K 14/70532; A61K 38/00; C07K 2319/30

(57) ABSTRACT

B7-H6-based compounds disclosed herein have favorable characteristics. Pharmaceutical compositions containing such a compound, the use of such compounds, and treatment with such pharmaceutical compositions are also disclosed. Methods for preparing a compound with an increased affinity for NKp30 are provided.

### FIELD OF THE INVENTION

The present disclosure relates to B7-H6-based compounds with favorable characteristics. Moreover, the present disclosure relates to pharmaceutical compositions comprising such a compound and the use of such compounds and such pharmaceutical compositions in medical treatment methods. Moreover, the present disclosure relates to methods for preparing a compound with an increased affinity for NKp30.

### BACKGROUND OF THE INVENTION

Despite advances in clinical treatment in the past decades, cancer is still one of the leading causes of death in the developed world. In recent years, hope for considerable progress has been raised by novel approaches that harness the power of the immune system, in particular by activating immune cells in the human body and directing them against the tumor cells. One cell type that shows great potential in this regard are natural killer cells (NK cells).

NK cells play a pivotal role in early host defense against infections and tumors. NK cells are innate immune cells that were discovered in the 1970s based on their ability to exert antitumor cell cytotoxicity without prior sensitization of the host. In contrast to T-cells that recognize distinct antigens via their variable T-cell receptors, the discrimination between healthy and stressed cells and consequently the antitumor response of NK cells is based on a sophisticated interplay between a multitude of germline-encoded activating and inhibitory receptors (Gonzales-Rodriguez et al., 2019; Chiossone et al., 2018).

Natural killer cells are innate lymphocytes that recognize discontinuity and danger in multiple tissue compartments by integrating positive and negative signals. The negative signals are generally mediated by the interaction between self MHC-I on tissues and either Killer-Immunoglobulin-like Receptor (KIR) family members or Natural Killer Group 2A (NKG2A) (Carlsten et al., 2019; Vivier et al., 2008). Positive signals are transduced via the interaction of an array of NK activation receptors including the Natural Cytotoxicity Receptors (NCRs; NKp30, NKp46, NKp44), NKG2D and DNAM-1 as well as costimulatory molecules including 4-1BB and their ligands (Koch et al., 2017; Morgado et al., 2011). For the NCRs and NKG2D, many of the ligands are ‘danger signals’ that are upregulated on stressed and diseased tissues including viral infected cells and tumor cells. Another mechanism by which NK cells are activated is the bridging of the low affinity activating FcγRIIIa (CD16a) on NK cells with cells opsonized with IgG antibodies or bispecific antibodies. Unlike the NCRs and NKG2D, signaling through FcγRIIIa is often more robust in resting NK cells but is modulated by multiple variables including functionally distinct polymorphic variants of FcγRIIIa as well as competition for binding with circulating IgG. Ultimately, the balance of activation and inhibitory signal determines whether an NK cell will become activated. As such, NK cells have an endogenous capacity to differentiate between healthy and diseased tissues.

Ultimately, NK cell activation results in target cell lysis via degranulation i.e. release of cytotoxic substances such as perforin and granzymes as well as in the production of proinflammatory cytokines and chemokines.

NK cells have shown great potential for the treatment of cancer by different approaches.

Several early clinical trials employing the adoptive transfer of wild-type or genetically modified (e.g. CAR) NK cells either alone or in combination with antibodies as a therapeutic modality for cancer have shown encouraging early results for hematological malignancies (Gonzales-Rodriguez et al., 2019; Burger et al., 2019; Rezvani et al., 2019).

Although adoptive cell therapy with ex vivo activated NK cells represents a promising approach, the logistic complexity has also driven the development of NK directed antibody-based approaches to cancer immunotherapy. In this respect antibodies have been developed that block the interaction between inhibitory receptors on NK cells, e.g. NKG2A or KIR2DL1, KIR2DL2 or KIR2DL3, and their ligands enabling immune cell activation (André et al., 2018; Kohrt et al., 2014; Benson et al., 2015). Furthermore, the vast majority of NK cells express the low affinity Fcγ receptor CD16a. CD16a-ligation of an antibody bound to its target cell induces potent NK cell degranulation (Bryceson et al., 2005). This process, referred to as antibody-dependent cellular cytotoxicity (ADCC) is considered as one important mode of action of many therapeutic antibodies (Seidel et al., 2013).

The capability of an antibody to elicit ADCC however, is affected by antigen densities on target cells. Due to the low affinity interaction of antibodies with CD16a, low antigen densities typically result in minor degrees of opsonization and consequently in limited induction of ADCC (Koch et al., 2017). Moreover, CD16a polymorphisms in humans have been described that result in different levels of ADCC, depending on the patient’s genotype. Finally, conventional therapeutic antibodies have to compete with serum immunoglobulins for CD16a binding, resulting in confined CD16a occupancy and restricted ADCC capacities (Ellwanger et al., 2019).

To overcome these inherent limitations of classical antibody therapies, bi- and multispecific NK cell engagers have been developed, in which one paratope binds to activating receptor CD16a with high affinities, while the other paratope is directed against a tumor-associated antigen (Koch et al., 2017; Rothe et al., 2015). In 2019, Vivier and co-workers described the efficient generation of trifunctional NK cell engagers (Gauthier et al., 2019). In their work, the authors employed two activating receptors of NK cells, NKp46 as well as CD16 (Fc-mediated) for effector cell engagement. In direct comparison with rituximab and Fc-engineered obinutuzumab in mouse in vivo studies, the developed NK cell engagers were more potent supporting the notion that this class of molecules might be promising therapeutic entities for tumor treatment.

A similar approach to target activating receptors on NK cells via antibodies relies on the exploitation of biological counterparts i.e. natural ligands of NK cell receptors. Combined with a tumor-targeting moiety in a bispecific format, effector cell engagers can readily be constructed. Such bispecific or trifunctional entities that form a bridge between an activating receptor on NK cells and a tumor associated antigen (TAA) on the tumor cell are referred to as NK cell engagers or immunoligands (Koch et al., 2017). Bispecific antibodies targeting a TAA (e.g. CD20) and NKp46, NKG2D and NKp30 (Peipp et al., 2015; Kellner et al., 2016) either via an antibody moiety or a recombinant form of the ectodomain of a ligand (e.g. ULBP2) (von Strandmann et al., 2006) have demonstrated potent target dependent cytotoxicity and cytokine release in vitro.

NKp30 is an activation receptor expressed on the majority of NK cells. Its cell bound ligand, B7-H6, is upregulated on tumor cells and absent on most normal cells. The other less well characterized ligand is HLA-B-associated transcript 3 (BAT3)/Bcl2-associated athanogene 6 (BAG6), which is expressed in the nucleus and can be transported to the plasma membrane or released in exosomes. Importantly, decreased NKp30 expression has been correlated with reduced survival in AML, and a lower number of NK cells expressing NKp30 were found in patients with gastric or breast cancer compared to healthy donors. Together, these data suggest that the NKp30 receptor axis may play an important role in tumor surveillance of different tumor entities. Therefore, potent strategies modulating the NKp30 axis may represent promising approaches to promote antitumor NK cell responses.

Despite the great potential of NK cell engagers based on natural ligands for NK cell activating receptors, in practice their use has been limited because the affinity of naturally available activating ligands for NK cells is insufficient for effective NK cell activation.

Accordingly, there is a need in the art for improved ways to treat cancer. Moreover, there is a need in the art for improved ways to activate NK cells. Moreover, there is a need in the art for NK cell activating ligands, such as ligands of NKp30, in particular for B7-H6-based ligands of NKp30, with improved characteristics, such as improved affinity, improved specificity, improved potency and/or efficacy for the killing of tumor cells, increased effects in the release of proinflammatory cytokines, improved pharmacokinetics, reduced side effects, increased therapeutic window and/or increased patient safety. Moreover, there is a need in the art to address the above-described needs by a “standardized” approach that can be widely used for activating NK cells (such as in combination with different immunoligands) and/or that is inexpensive and allows for fast synthetic access.

The present disclosure overcomes the above-described problems and addresses the above-described needs.

### SUMMARY OF THE INVENTION

The present disclosure addresses the needs described above in the section “Background of the Invention” by the different aspects and embodiments described below.

The present invention is, in part, based on the surprising observation that compounds comprising an affinity-matured, B7-H6-based domain as described in the present disclosure exhibit various advantageous effects. For example, advantageous effects can include (but are not limited to) a high affinity for NKp30, a high Kon rate for NKp30 binding, a low Koff rate for NKp30 binding, a high efficiency in activating NK cells, high efficiency in inducing cytokine release (interferon-y, TNF-α), (in particular in an immunoligand context) enhanced cytotoxicity (e.g. with regard to potency and/or efficacy) and improved serial killing (i.e. tumor cell killing per time) and improved manufacturability. If used in combination with an Fc region capable of FcyRIIIa binding, this cytotoxicity is further enhanced.

In one aspect, the present disclosure relates to a compound comprising

(a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3),

wherein
X₁ is I, L, T, V, H, W or Y;
X₂ is G;
X₃ is W or Y;
X₄ is G;
X₅ is V or I;
X₆ is T;
X₇ is Y, F or L;
X₈ is K;

(b) a protein domain which consists of an amino acid sequence that is at least 75% identical to the amino acid sequence of the protein domain of (a); or
(c) a protein domain which is a fragment of the protein domain of (a) or (b).

In another aspect, the present disclosure relates to a compound comprising

(a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3),

wherein
X₁ is H, E, S, T, I or W;
X₂ is G;
X₃ is Y, W or F;
X₄ is G or D;
X₅ is V, F or I;
X₆ is T;
X₇ is Y or L;
X₈ is K;

(b) a protein domain which consists of an amino acid sequence that is at least 75% identical to the amino acid sequence of the protein domain of (a); or
(c) a protein domain which is a fragment of the protein domain of (a) or (b).

In another aspect, the present disclosure relates to a compound comprising

(a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3),

wherein
X₁ is E, S, H, T, L or Q;
X₂ is G;
X₃ is W, Y or F;
X₄ is G, A, D or Q;
X₅ is V, I or F;
X₆ is T;
X₇ is Y, L or V;
X₈ is K;

(b) a protein domain which consists of an amino acid sequence that is at least 75% identical to the amino acid sequence of the protein domain of (a); or
(c) a protein domain which is a fragment of the protein domain of (a) or (b).

In another aspect, the present disclosure relates to a compound comprising

(a) a protein domain which consists of the amino acid sequence of any one of the following sequences:

| | |
|---|---|
| SEQ ID NO | Amino acid sequence |
| SEQ ID NO: 4 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 5 | DLKVEMMAGGTQITPLNDNVTIFCNIFYSQPLNITWMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 6 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVFVTPYKAQGTVQLEVVASPAS |
| SEQ ID NO: 7 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 8 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 9 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 10 | DLKVEMMAGGTQITPLNDNVTIFCNIFYSQPLNITHMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 11 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVIVTPLKAQGTVQLEVVASPAS |
| SEQ ID NO: 12 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 13 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPYKAQGTVQLEVVASPAS |
| SEQ ID NO: 14 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVIVTPFKAQGTVQLEVVASPAS |
| SEQ ID NO: 15 | DLKVEMMAGGTQITPLNDNVTIFCNIFYSQPLNITEMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 17 | DLKVEMMAGGTQITPLNDNVTIFCNIFYSQPLNITTMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 18 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 19 | DLKVEMMAGGTQITPLNDNVTIFCNIFYSQPLNITLMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 20 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPLKAQGTVQLEVVASPAS |
| SEQ ID NO: 21 | DLKVEMMAGGTQITPLNDNVTIFCNIFYSQPLNITYMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 22 | DLKVEMMAGGTQITPLNDNVTIFCNIFYSQPLNITQMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 23 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVFVTPLKAQGTVQLEVVASPAS |
| SEQ ID NO: 24 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 25 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 26 | DLKVEMMAGGTQITPLNDNVTIFCNIFYSQPLNITEMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 27 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 28 | DLKVEMMAGGTQITPLNDNVTIFCNIFYSQPLNITWMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 29 | DLKVEMMAGGTQITPLNDNVTIFCNIFYSQPLNITTMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 30 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 31 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 32 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 33 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVIVTPLKAQGTVQLEVVASPAS |
| SEQ ID NO: 34 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 35 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVVVTPVKAQGTVQLEVVASPAS |
| SEQ ID NO: 36 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 37 | DLKVEMMAGGTQITPLNDNVTIFCNIFYSQPLNITSMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 38 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 39 | DLKVEMMAGGTQITPLNDNVTIFCNIFYSQPLNITHMGITW FWKSLTFDKEVKVFEFYADHQEAIRPGAIVSPWRLKSGDAS LRLPGIQLEEAGEYRCEVVVTPYKAQGTVQLEVVASPAS |
| SEQ ID NO: 40 | DLKVEMMAGGTQITPLNDNVTIFCNIFYSQPLNITSMGITW LWKSLTFDKEVKVFEFWGDHQEAFRPGAIVSPWRLKSGDAS LRLPGIQLEEAGEYRCEVFVTPYKAQGTVQLEVVASPAS |
| SEQ ID NO: 41 | DLKVEMMAGGTQITPLNDNVTIFCNIFYFQPLNITEMGITW FWKSLTFDKEVKVFEFFGDHQEAFRPGAIVSPWRLKSGDAS LRLPGIQLEEAGEYRCEVIVTPLKAQGTVQLEVVASPAS |
| SEQ ID NO: 42 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVIVTPYKAQGTVQLEVVTSPAS |
| SEQ ID NO: 43 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCGVVVTPVKAQGTVQLEVVASPAS |
| SEQ ID NO: 44 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVVVTPLKAQGTAQLEVVASPAS |
| SEQ ID NO: 45 | DLKVEMMAGGTQITPLNDNVTIFCNIFYSQPLNITLMGITW FWKSLTFDKEVKVFEFWGDHQEAFRPGAIVSPWGLKSGDAS LRLPGIQLEEAGEYRCEVVVTPLKAQGTVQLEVVASPAS |
| SEQ ID NO: 46 | DLKVEMMAGGTQITPLNDNVTIFCNIFYSQPLNITTMGITW FWKSLTFDKEVKVFEFWQDHQEAFRPGAIVSPWRLKSGDAS LRLPGIQLEEAGEYRCEVVVTPLKAQGTVQLEVVASPAS |
| SEQ ID NO: 47 | DLKVEMMAGGTQITPLNDNVTIFCNIFYSQPLNITHMGITW LWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 48 | DLKVEMMAGGTQITPLNDNVTIFCNIFYSQPLNITSMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 49 | DLKVEMMAGGTQITPLNDNVTIFCNIFYSQPLNITTMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 50 | DLKVEMMAGGTQITPLNDNVTIFCNIFYSQPLNITEMGITW FWKSLTFDKEVKVFEFYGDHQEAFXPGAIVSPWRLKSGDAS LXLPGIQLEEAGEYRCEVVVTPLKAQGTVQLEVVASPAS |

(b) a protein domain which consists of an amino acid sequence that is identical to at least one sequence listed under (a) with the only difference that in up to 30 occasions an amino acid is individually added, replaced by another amino acid or deleted in the amino acid sequence of the protein domain of (b) compared to said at least one sequence listed under (a).

(c) a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 30 occasions an amino acid is individually added, replaced by another amino acid or deleted in the amino acid sequence of the protein domain of (c) compared to said at least one sequence listed under (a).

In another aspect, the present disclosure relates to a pharmaceutical composition comprising the compound according to the present disclosure.

In another aspect, the present disclosure relates to a compound according to the present disclosure or a pharmaceutical composition according to the present disclosure for use as a medicament or for use in the treatment of a disease as defined below.

In another aspect, the present disclosure relates to a method for treating a disease in a patient in need thereof, comprising the step of administering to said patient a therapeutically effective amount of the compound according to the present disclosure or the pharmaceutical composition according to the present disclosure.

In another aspect, the present disclosure relates to the use of the compound according to the present disclosure or of the pharmaceutical composition according to the present disclosure for the manufacture of a medicament, preferably for the manufacture of a medicament for the treatment of a disease or disorder as defined below.

In another aspect, the present disclosure relates to a method for preparing a compound with an increased affinity for NKp30 compared to a compound comprising a protein domain with the amino acid sequence of SEQ ID NO: 2:

DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPAS (SEQ ID N
O: 2),

wherein said method comprises preparing a compound comprising a variant of the protein domain with the amino acid sequence of SEQ ID NO: 2 in which at least one of the following amino acid replacements has been carried out compared to the sequence of SEQ ID NO: 2:
replacement of X₁ by I, L, T, V, H, W, Y, E or Q;
replacement of X₃ by W or Y;
replacement of X₄ by D, A or Q;
replacement of X₅ by I or F;
replacement of X₇ by Y, F or V,
wherein X₁, X₃, X₄, X₅ and X₇ refer to the following positions within the sequence of SEQ ID NO: 2:

DLKVEMMAGGTQITPLNDNVTIFCNIFYSQPLNITX₁ MGITWFWKSLTFD
KEVKVFEFX₃X₄ DHQEAFRPGAIVSPWRLKSGDASLRLPGIQLEEAGEYR
CEVX₅ VTPX₇ KAQGTVQLEVVASPAS.

### BRIEF DESCRIPTION OF THE FIGURES

In the following, reference is made to the figures. All methods referred to in the figure descriptions below were carried out as described in detail in the examples.

FIG. 1 shows data from an experiment with bispecific molecules comprising a targeting domain (antibody fragment directed against a tumor-associated antigen) and a variant B7H6 IgV-like domain for testing if the B7-H6 IgV-like domain is sufficient to elicit NK cell-mediated tumor cell lysis. (A) Scheme of generated immunoligands for NK cell redirection consisting of the B7-H6 IgV-like domain (ΔB7-H6; dark blue) and the humanized Fab of Cetuximab (hu225) in an effector-silenced IgG1 SEED backbone comprising amino acid exchanges L234A, L235A, P329G. VH and VL colored in greencyan, antibody backbone in green, SEED GA and AG in CH3 domains indicated by deepteal coloring. (B) Schematic depiction of the design of B7-H6-derived immunoligands. The N-terminal Ig-like V-type domain of B7-H6 was fused to the AG chain of a SEED-derived heavy chain, while humanized Cetuximab was utilized on the GA chain as well as the light chain. Sequences of respective chains are given for the wild type B7-H6-derived SEEDbody. (C) BLI (biolayer interferometry) analysis of wild-type (wt) ΔB7-H6 immunoligand binding to NKp30. (D) Killing property of AB7-H6_wt-SEED-PGLALA (■) was compared to one-armed (oa)_hu225-SEED activating FcγRIIIa (◆) and Fc silenced oa_hu225-SEED-PGLALA lacking the B7-H6 IgV-like domain (◇) as a control molecule in standard 4 h ⁵¹Cr release experiments using freshly isolated human NK cells of healthy donors and A431 tumor cells in an effector-to-target cell (E:T) ratio of 10:1. Normalized percent tumor cell lysis of 3 independent experiments with different donors is shown as mean ± SEM. *** p < 0.001; ** p < 0.01 of oa_hu225-SEED vs. ΔB7-H6_wt-SEED-PGLALA.

FIG. 2 is a graphic depiction of the frequency of different amino acids at specific positions of the affinity-maturated ΔB7-H6 variants, split up into groups of variants based on their affinity for NKp30. The sequences in the subpanels (A)-(C) are based on ΔB7-H6 variants with an affinity for NKp30 of (A) KD < 40 nM, (B) KD 40-120 nM and (C) KD > 120 nM, respectively. The amino acids at the different positions show the amino acid exchange (or maintenance) of the variants compared to ΔB7-H6 wt at the respective position (Ser60, Gly62, Phe82, Gly83, Val125, Thr127, Leu129, Lys130), wherein the size of the amino acid encoding letter-code correlates with the mutation frequency for the specific amino acid at this position.

FIG. 3 shows data from an experiment to test the cytotoxic activity of affinity-optimized ΔB7-H6-based NK cell engagers. Standard 4h ⁵¹Cr release assays were performed with A431 cells expressing high EGFR levels (left) and A549 cells expressing low EGFR levels (right) using human PBMCs at an E:T ratio of 80:1 (A) or NK cells at an E:T ratio of 10:1 (B) to analyze dose-dependent killing of the leading ΔB7-H6 SEED-PGLALA NK cell engagers. The affinity-matured ΔB7-H6 variants were compared to wild-type ΔB7-H6 wt_hu225-SEED-PGLALA (grey) and a control molecule lacking ΔB7-H6, but still binding one-armed (oa) to EGFR via the humanized Cetuximab Fab-fragment (oa_hu225-SEED-PGLALA; dotted black line). To allow comparison of the results, data of each experiment were normalized. Graphs show normalized means ± SEM of three experiments performed with PBMCs or NK cell from different donors. (C) Specificity of A431 lysis mediated by the leading 4 NK cell engagers (applied at 0.6 nM) was verified by either blocking EGFR binding by pre-incubation of A431 cells with 50 µg/ml of the Fc-silenced oa_hu225-SEED-PGLALA (left graph) or NKp30 binding by pre-incubation of NK cells with 50 µg/ml anti-NKp30 antibody (right graph). Graphs show percentage of inhibited lysis as means ± SEM of 4 individual experiments. *** p < 0.001; * p < 0.05 compared to NK cells only (left graph) or Cetuximab (right graph).

FIG. 4 is a graph in which the affinity of the different ΔB7-H6 variants for NKp30 was plotted against the killing efficacy (EC50_{A431}). Every data point depicts a single ΔB7-H6 variant. Outliers marked in light grey.

FIG. 5 shows data of NK cell activation, IFN-γ release and TNF-α release for selected ΔB7-H6 SEED-PGLALA immunoligands. Human NK cells were co-cultured for 24 h with A431 cells at an E:T ratio of 5:1 prior to analysis of NK cell activation (A), IFN-γ (B) and TNF-α (C) release. Affinity-matured ΔB7-H6 NK cell engagers and oa_hu225-SEED-PGLALA were compared to wild-type ΔB7-H6_wt-SEED-PGLALA (all at 85 nM). Percentage of CD69-positive NK cells was determined by double staining of viable NK cells with CD56-PE and CD69-APC using flow cytometric analyses. Human cytokine HTRF kits were utilized for the quantification of IFN-γ and TNF-α release of NK cells. Graphs show box and whiskers plots as superimposition with dot plots of six (A) and eight (B, C) individual experiments, respectively. *** p < 0.001; ** p < 0.01, * p < 0.05, n.s. not significant compared to ΔB7-H6_wt-SEED-PGLALA.

FIG. 6 shows representative dose response curves of one healthy donor for NK cell mediated IFN-γ release obtained with various ΔB7-H6 SEED-PGLALA immunoligands.

FIG. 7 shows representative dose response curves of one healthy donor for NK cell mediated TNF-α release obtained with various ΔB7-H6 SEED-PGLALA immunoligands.

FIG. 8 shows data obtained in an experiment to examine synergistic cytotoxic activity by concomitant NKp30 and FcγRIIIa engagement of ΔB7-H6-based immunoligands with functional IgG1 Fc. The highest affinity variant for NKp30 binding, S3#18, was tested in 4h ⁵¹Cr release assays with A431 cells and NK cells at an E:T ratio of 10:1 to compare dose-dependent killing of (A) the Fc-silenced ΔB7-H6 S3#18-SEED-PGLALA (dark green, marked with #), oa_hu225-SEED (grey, marked with †) lacking ΔB7-H6 and ΔB7-H6 S3#18-SEED with a functional Fc (light green) or (B) Cetuximab (black) and ΔB7-H6 S3#18 SEED both carrying a functional Fc. Fc-silenced oa_hu225-SEED-PGLALA (dotted black line) lacking also ΔB7-H6 and thus NKp30 activation was used as control. Graph shows percentage of lysis as means ± SEM of 7 independent experiments. Cytokine release of IFN-γ (C) and TNF-α (D) as well as NK cell activation (E) for effector silenced and effector competent ΔB7-H6 S3#18-SEED molecules as well as effector silenced and effector competent oa_hu225-SEED and Cetuximab (all at 85 nM). Cytokine release was analyzed after 24 h incubation of purified NK cells with A431 cells at an E:T ratio of 5:1 with human cytokine HTRF kits. Percentage of CD69-positive NK cells was determined by double staining of viable NK cells with CD56-PE and CD69-APC using flow cytometry. Human cytokine HTRF kits were utilized for the quantification of IFN-γ and TNF-α release of NK cells. Profiles show box and whiskers plots as superimpositions with dot plots of eight independent experiments. *** p < 0.001; ** p < 0.01, * p < 0.05, n.s. not significant compared to the ΔB7-H6 S3#18-SEED-PGLALA.

SUMMARY OF SEQUENCES

| | | |
|---|---|---|
| SEQ ID NO | Identifier/Description | Amino acid sequence |
| SEQ ID NO: 1 | Human B7-H6* | MTWRAAASTCAALLILLWALTTEGDLKVEMMAGGTQ ITPLNDNVTIFCNIFYSQPLNITSMGITWFWKSLTF DKEVKVFEFFGDHQEAFRPGAIVSPWRLKSGDASLR LPGIQLEEAGEYRCEVVVTPLKAQGTVQLEVVASPA SRLLLDQVGMKENEDKYMCESSGFYPEAINITWEKQ TQKFPHPIEISEDVITGPTIKNMDGTFNVTSCLKLN SSQEDPGTVYQCVVRHASLHTPLRSNFTLTAARHSL SETEKTDNFSIHWWPISFIGVGLVLLIVLIPWKKIC NKSSSAYTPLKCILKHWNSFDTQTLKKEHLIFFCTR AWPSYQLQDGEAWPPEGSVNINTIQQLDVFCRQEGK |
| | | WSEVPYVQAFFALRDNPDLCQCCRIDPALLTVTSGK SIDDNSTKSEKQTPREHSDAVPDAPILPVSPIWEPP PATTSTTPVLSSQPPTLLLPLQ |
| SEQ ID NO: 2 | ΔB7-H6 wt^{#} | DLKVEMMAGGTQITPLNDNVTIFCNIFYSQPLNITS MGITWFWKSLTFDKEVKVFEFFGDHQEAFRPGAIVS PWRLKSGDASLRLPGIQLEEAGEYRCEVVVTPLKAQ GTVQLEVVASPAS |
| SEQ ID NO: 3 | ΔB7-H6 sequence with modification sites X₁-X₈^{†} | DLKVEMMAGGTQITPLNDNVTIFCNIFYSQPLNITX₁ MX₂ ITWFWKSLTFDKEVKVFEFX₃X₄ DHQEAFRPGAI VSPWRLKSGDASLRLPGIQLEEAGEYRCEVX₅ VX₆ PX₇X₈ AQGTVQLEVVASPAS |
| SEQ ID NO: 4 | ΔB7-H6_S3#1 | DLKVEMMAGGTQITPLNDNVTIFCNIFYSQPLNITT MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 5 | ΔB7-H6_S3#2 | DLKVEMMAGGTQITPLNDNVTIFCNIFYSQPLNITW MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 6 | ΔB7-H6_S3#3 | DLKVEMMAGGTQITPLNDNVTIFCNIFYSQPLNITH MGITWFWKSLTFDKEVKVFEFFGDHQEAFRPGAIVS PWRLKSGDASLRLPGIQLEEAGEYRCEVFVTPYKAQ GTVQLEVVASPAS |
| SEQ ID NO: 7 | ΔB7-H6_S3#4 | DLKVEMMAGGTQITPLNDNVTIFCNIFYSQPLNITI MGITWFWKSLTFDKEVKVFEFWGDHQEAFRPGAIVS PWRLKSGDASLRLPGIQLEEAGEYRCEVVVTPLKAQ GTVQLEVVASPAS |
| SEQ ID NO: 8 | ΔB7-H6_S3#5 | DLKVEMMAGGTQITPLNDNVTIFCNIFYSQPLNITV MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 9 | ΔB7-H6_S3#6 | DLKVEMMAGGTQITPLNDNVTIFCNIFYSQPLNITH MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 10 | ΔB7-H6_S3#7 | DLKVEMMAGGTQITPLNDNVTIFCNIFYSQPLNITH MGITWFWKSLTFDKEVKVFEFYGDHQEAFRPGAIVS PWRLKSGDASLRLPGIQLEEAGEYRCEVVVTPYKAQ GTVQLEVVASPAS |
| SEQ ID NO: 11 | ΔB7-H6_S3#8 | DLKVEMMAGGTQITPLNDNVTIFCNIFYSQPLNITH MGITWFWKSLTFDKEVKVFEFWGDHQEAFRPGAIVS PWRLKSGDASLRLPGIQLEEAGEYRCEVIVTPLKAQ GTVQLEVVASPAS |
| SEQ ID NO: 12 | ΔB7-H6_S3#9 | DLKVEMMAGGTQITPLNDNVTIFCNIFYSQPLNITH MGITWFWKSLTFDKEVKVFEFFGDHQEAFRPGAIVS PWRLKSGDASLRLPGIQLEEAGEYRCEVVVTPYKAQ GTVQLEVVASPAS |
| SEQ ID NO: 13 | ΔB7-H6_S3#10 | DLKVEMMAGGTQITPLNDNVTIFCNIFYSQPLNITS MGITWFWKSLTFDKEVKVFEFYGDHQEAFRPGAIVS PWRLKSGDASLRLPGIQLEEAGEYRCEVFVTPYKAQG TVQLEVVASPAS |
| SEQ ID NO: 14 | ΔB7-H6_S3#11 | DLKVEMMAGGTQITPLNDNVTIFCNIFYSQPLNITL MGITWFWKSLTFDKEVKVFEFWGDHQEAFRPGAIVS PWRLKSGDASLRLPGIQLEEAGEYRCEVIVTPFKAQ GTVQLEVVASPAS |
| SEQ ID NO: 15 | ΔB7-H6_S3#12 | DLKVEMMAGGTQITPLNDNVTIFCNIFYSQPLNITE MGITWFWKSLTFDKEVKVFEFFGDHQEAFRPGAIVS PWRLKSGDASLRLPGIQLEEAGEYRCEVVVTPYKAQ GTVQLEVVASPAS |
| SEQ ID NO: 16 | ΔB7-H6_S3#13 | DLKVEMMAGGTQITPLNDNVTIFCNIFYSQPLNITS MIITWFWKSLTFDKEVKVFEFYGDHQEAFRPGAIVS PWRLKSGDASLRLPGIQLEEAGEYRCEVVVTPLKAQ GTVQLEVVASPAS |
| SEQ ID NO: 17 | ΔB7-H6_S3#14 | DLKVEMMAGGTQITPLNDNVTIFCNIFYSQPLNITT MGITWFWKSLTFDKEVKVFEFYGHQEAFRPGAIVS PWRLKSGDASLRLPGIQLEEAGEYRCEVVVTPYKAQ GTVQLEVVASPAS |
| SEQ ID NO: 18 | ΔB7-H6_S3#15 | DLKVEMMAGGTQITPLNDNVTIFCNIFYSQPLNITI MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 19 | ΔB7-H6_S3#16 | DLKVEMMAGGTQITPLNDNVTIFCNIFYSQPLNITL MGITWFWKSLTFDKEVKVFEFYGDHQEAFRPGAIVS PWRLKSGDASLRLPGIQLEEAGEYRCEVVVTPYKAQ GTVQLEVVASPAS |
| SEQ ID NO: 20 | ΔB7-H6_S3#17 | DLKVEMMAGGTQITPLNDNVTIFCNIFYSQPLNITS MGITWFWKSLTFDKEVKVFEFYGDHQEAFRPGAIVS PWRLKSGDASLRLPGIQLEEAGEYRCEVFVTPLKAQ GTVQLEVVASPAS |
| SEQ ID NO: 21 | ΔB7-H6_S3#18 | DLKVEMMAGGTQITPLNDNVTIFCNIFYSQPLNITY MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 22 | ΔB7-H6_S3#19 | DLKVEMMAGGTQITPLNDNVTIFCNIFYSQPLNITQ MGITWFWKSLTFDKEVKVFEFWGDHQEAFRPGAIVS |
| | | PWRLKSGDASLRLPGIQLEEAGEYRCEVVVTPYKAQ GTVQLEVVASPAS |
| SEQ ID NO: 23 | ΔB7-H6_S3#20 | DLKVEMMAGGTQITPLNDNVTIFCNIFYSQPLNITH MGITWFWKSLTFDKEVKVFEFWGDHQEAFRPGAIVS PWRLKSGDASLRLPGIQLEEAGEYRCEVFVTPLKAQ GTVQLEVVASPAS |
| SEQ ID NO: 24 | ΔB7-H6_S3#21 | DLKVEMMAGGTQITPLNDNVTIFCNIFYSQPLNITL MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 25 | ΔB7-H6_S3#22 | DLKVEMMAGGTQITPLNDNVTIFCNIFYSQPLNITE MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 26 | ΔB7-H6_S3#23 | DLKVEMMAGGTQITPLNDNVTIFCNIFYSQPLNITE MGITWFWKSLTFDKEVKVFEFYGDHQEAFRPGAIVS PWRLKSGDASLRLPGIQLEEAGEYRCEVVVTPYKAQ GTVQLEVVASPAS |
| SEQ ID NO: 27 | ΔB7-H6_S3#24 | DLKVEMMAGGTQITPLNDNVTIFCNIFYSQPLNITI MGITWFWKSLTFDKEVKVFEFYGDHQEAFRPGAIVS PWRLKSGDASLRLPGIQLEEAGEYRCEVVVTPYKAQ GTVQLEVVASPAS |
| SEQ ID NO: 28 | ΔB7-H6_S3#25 | DLKVEMMAGGTQITPLNDNVTIFCNIFYSQPLNITW MGITWFWKSLTFDKEVKVFEFYGDHQEAFRPGAIVS PWRLKSGDASLRLPGIQLEEAGEYRCEVVVTPYKAQ GTVQLEVVASPAS |
| SEQ ID NO: 29 | ΔB7-H6_S3#26 | DLKVEMMAGGTQITPLNDNVTIFCNIFYSQPLNITT MGITWFWKSLTFDKEVKVFEFWDDHQEAFRPGAIVS PWRLKSGDASLRLPGIQLEEAGEYRCEVVVTPYKAQ GTVQLEVVASPAS |
| SEQ ID NO: 30 | ΔB7-H6_S3#27 | DLKVEMMAGGTQITPLNDNVTIFCNIFYSQPLNITT MGITWFWKSLTFDKEVKVFEFWGDHQEAFRPGAIVS PWRLKSGDASLRLPGIQLEEAGEYRCEVIVTPYKAQ GTVQLEVVASPAS |
| SEQ ID NO: 31 | ΔB7-H6_S3#28 | DLKVEMMAGGTQITPLNDNVTIFCNIFYSQPLNITH MGITWFWKSLTFDKEVKVFEFWGDHQEAFRPGAIVS PWRLKSGDASLRLPGIQLEEAGEYRCEVVVTPLKAQ GTVQLEVVASPAS |
| SEQ ID NO: 32 | ΔB7-H6_S3#29 | DLKVEMMAGGTQITPLNDNVTIFCNIFYSQPLNITV MGITWFWKSLTFDKEVKVFEFWGDHQEAFRPGAIVS PWRLKSGDASLRLPGIQLEEAGEYRCEVIVTPYKAQ GTVQLEVVASPAS |
| SEQ ID NO: 33 | ΔB7-H6_S3#30 | DLKVEMMAGGTQITPLNDNVTIFCNIFYSQPLNITE MGITWFWKSLTFDKEVKVFEFWGDHQEAFRPGAIVS PWRLKSGDASLRLPGIQLEEAGEYRCEVIVTPLKAQ GTVQLEVVASPAS |
| SEQ ID NO: 34 | ΔB7-H6_S3#31 | DLKVEMMAGGTQITPLNDNVTIFCNIFYSQPLNITI MGITWFWKSLTFDKEVKVFEFYGDHQEAFRPGAIVS PWRLKSGDASLRLPGIQLEEAGEYRCEVVVTPLKAQ GTVQLEVVASPAS |
| SEQ ID NO: 35 | ΔB7-H6_S3#32 | DLKVEMMAGGTQITPLNDNVTIFCNIFYSQPLNITE MGITWFWKSLTFDKEVKVFEFWGDHQEAFRPGAIVS PWRLKSGDASLRLPGIQLEEAGEYRCEVVVTPVKAQ GTVQLEVVASPAS |
| SEQ ID NO: 36 | ΔB7-H6_S2#1 | DLKVEMMAGGTQITPLNDNVTIFCNIFYSQPLNITS MGITWFWKSLTFDKEVKVFEFYGDHQEAFRPGAIVS PWRLKSGDASLRLPGIQLEEAGEYRCEVVVTPYKAQ GTVQLEVVASPAS |
| SEQ ID NO: 37 | ΔB7-H6_S2#2 | DLKVEMMAGGTQITPLNDNVTIFCNIFYSQPLNITS MGITWFWKSLTFDKEVKVFEFWDDHQEAFRPGAIVS PWRLKSGDASLRLPGIQLEEAGEYRCEVVVTPYKAQ GTVQLEVVASPAS |
| SEQ ID NO: 38 | ΔB7-H6_S2#3 | DLKVEMMAGGTQITPLNDNVTIFCNIFYSQPLNITT MGITWFWKSLTFDKEVKVFEFWGDHQEAFRPGAIVS PWRLKSGDASLRLPGIQLEEAGEYRCEVVVTPLKAQ GTVQLEVVASPAS |
| SEQ ID NO: 39 | ΔB7-H6_S2#4 | DLKVEMMAGGTQITPLNDNVTIFCNIFYSQPLNITH MGITWFWKSLTFDKEVKVFEFYADHQEAIRPGAIVS PWRLKSGDASLRLPGIQLEEAGEYRCEVVVTPYKAQ GTVQLEVVASPAS |
| SEQ ID NO: 40 | ΔB7-H6_S2#5 | DLKVEMMAGGTQITPLNDNVTIFCNIFYSQPLNITS MGITWLWKSLTFDKEVKVFEFWGDHQEAFRPGAIVS PWRLKSGDASLRLPGIQLEEAGEYRCEVFVTPYKAQ GTVQLEVVASPAS |
| SEQ ID NO: 41 | ΔB7-H6_S2#6 | DLKVEMMAGGTQITPLNDNVTIFCNIFYFQPLNITE MGITWFWKSLTFDKEVKVFEFFGDHQEAFRPGAIVS PWRLKSGDASLRLPGIQLEEAGEYRCEVIVTPLKAQ GTVQLEVVASPAS |
| SEQ ID NO: 42 | ΔB7-H6_S2#7 | DLKVEMMAGGTQITPLNDNVTIFCNIFYSQPLNITS MGITWFWKSLTFDKEVKVFEFYGDHQEAFRPGAIVS PWRLKSGDASLRLPGIQLEEAGEYRCEVIVTPYKAQ GTVQLEVVTSPAS |
| SEQ ID NO: 43 | ΔB7-H6_S2#8 | DLKVEMMAGGTQITPLNDNVTIFCNIFYSQPLNITT MGITWFWKSLTFDKEVKVFEFWGDHQEAFRPGAIVS |
| | | PWRLKSGDASLRLPGIQLEEAGEYRCGVVVTPVKAQ GTVQLEVVASPAS |
| SEQ ID NO: 44 | ΔB7-H6_S2#9 | DLKVEMMAGGTQITPLNDNVTIFCNIFYSQPLNITS MGITWFWKSLTFDKEVKVFEFYGDHQEAFRPGAIVS PWRLKSGDASLRLPGIQLEEAGEYRCEVVVTPLKAQ GTAQLEVVASPAS |
| SEQ ID NO: 45 | ΔB7-H6_S2#10 | DLKVEMMAGGTQITPLNDNVTIFCNIFYSQPLNITL MGITWFWKSLTFDKEVKVFEFWGDHQEAFRPGAIVS PWGLKSGDASLRLPGIQLEEAGEYRCEVVVTPLKAQ GTVQLEVVASPAS |
| SEQ ID NO: 46 | ΔB7-H6_S2#11 | DLKVEMMAGGTQITPLNDNVTIFCNIFYSQPLNITT MGITWFWKSLTFDKEVKVFEFWQDHQEAFRPGAIVS PWRLKSGDASLRLPGIQLEEAGEYRCEVVVTPLKAQ GTVQLEVVASPAS |
| SEQ ID NO: 47 | ΔB7-H6_S2#12 | DLKVEMMAGGTQITPLNDNVTIFCNIFYSQPLNITH MGITWLWKSLTFDKEVKVFEFYGDHQEAFRPGAIVS PWRLKSGDASLRLPGIQLEEAGEYRCEVVVTPLKAQ GTVQLEVVASPAS |
| SEQ ID NO: 48 | ΔB7-H6_S2#13 | DLKVEMMAGGTQITPLNDNVTIFCNIFYSQPLNITS MGITWFWKSLTFDKEVKVFEFWGDHQEAFRPGAIVS PWRLKSGDASLRLPGIQLEEAGEYRCEVVVTPYKAQ GTVQLEVVASPAS |
| SEQ ID NO: 49 | ΔB7-H6_S2#14 | DLKVEMMAGGTQITPLNDNVTIFCNIFYSQPLNITT MGITWFWKSLTFDKEVKVFEFFGDHQEAFRPGAIVS PWRLKSGDASLRLPGIQLEEAGEYRCEVVVTPYKAQ GTVQLEVVASPAS |
| SEQ ID NO: 50 | ΔB7-H6_S2#15 | DLKVEMMAGGTQITPLNDNVTIFCNIFYSQPLNITE MGITWFWKSLTFDKEVKVFEFYGDHQEAFXPGAIVS PWRLKSGDASLXLPGIQLEEAGEYRCEVVVTPLKAQ GTVQLEVVASPAS |
| SEQ ID NO: 51 | ΔB7-H6 sequence with modification sites X₁, X₃, X₄, X₅ and X₇ | DLKVEMMAGGTQITPLNDNVTIFCNIFYSQPLNITX₁ MGITWFWKSLTFDKEVKVFEFX₃X₄ DHQEAFRPGAIV SPWRLKSGDASLRLPGIQLEEAGEYRCEVX₅ VTPX₇ K AQGTVQLEVVASPAS |
| SEQ ID NO: 52 | ΔB7-H6 sequence with modification sites X₆ and X₈ | DLKVEMMAGGTQITPLNDNVTIFCNIFYSQPLNITS MGITWFWKSLTFDKEVKVFEFFGDHQEAFRPGAIVS PWRLKSGDASLRLPGIQLEEAGEYRCEVVVX₆ PLX₈ A QGTVQLEVVASPAS |
| SEQ ID NO: 53 | ΔB7-H6 sequence with modification | DLKVEMMAGGTQITPLNDNVTIFCNIFYSQPLNITS MX₂ ITWFWKSLTFDKEVKVFEFFGDHQEAFRPGAIVS PWRLKSGDASLRLPGIQLEEAGEYRCEVVVX₆ PLX₈ A QGTVQLEVVASPAS |
| | sites X₂, X₆ and X₈ | |

^{∗} Residues Ser60, Gly62, Phe82, Gly83, Val125, Thr127, Leu129 and Lys130 are shown in bold.
^{#} SEQ ID NO: 2 is a sequence comprising the IgV-like domain of human B7-H6 (plus additional 2 amino acids of the human B7-H6 sequence (DL) at the N-terminus and additional 7 amino acids of the human B7-H6 sequence (VVASPAS) at the C-terminus of the IgV like domain). Residues corresponding to Ser60, Gly62, Phe82, Gly83, Val125, Thr127, Leu129 and Lys130 of full-length human B7-H6 are shown in bold.
^{†} In the sequence of SEQ ID NO: 3, X₁ is at the position corresponding to Ser60 in human full-length B7-H6; X₂ is at the position corresponding to Gly62 in human full-length B7-H6; X₃ is at the position corresponding to Phe82 in human full-length B7-H6; X₄ is at the position corresponding to Gly83 in human full-length B7-H6; X₅ is at the position corresponding to Val125 in human full-length B7-H6; X₆ is at the position corresponding to Thr127 in human full-length B7-H6; X₇ is at the position corresponding to Leu129 in human full-length B7-H6; X₈ is at the position corresponding to Lys130 in human full-length B7-H6.

### DETAILED DESCRIPTION OF CERTAIN EMBODIMENTS

Although the present disclosure is described in detail above and below, it is to be understood that this disclosure is not limited to the particular methodologies, protocols and reagents described by the present disclosure, as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, certain elements of the present disclosure will be described in more detail, including the description of specific embodiments. However, the variously described examples and preferred embodiments should not be construed to limit the present disclosure to only the explicitly described embodiments. This description should be understood to support and encompass embodiments which combine the explicitly described embodiments with any number of the disclosed and/or preferred elements and in any manner. Furthermore, any permutations and combinations of all described elements in this application should be considered disclosed by the description of the present application except for where this leads to logical contradictions or the context indicates otherwise.

Unless defined otherwise herein, scientific and technical terms used in connection with the present disclosure shall have the meanings that are commonly understood by those of ordinary skill in the art. Generally, nomenclatures and techniques referred to in the present disclosure, e.g. nomenclatures and techniques of organic chemistry, chemical synthesis, biology, medicinal and pharmaceutical chemistry, medicine, pharmacology or toxicology, are those well-known and commonly used in the art. The methods and techniques of the present disclosure are generally performed according to conventional methods well-known in the art and as described in the references cited and discussed throughout the present disclosure unless otherwise indicated.

According to one aspect, the present disclosure relates to a compound comprising

(a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3)

wherein
X₁ is I, L, T, V, H, W or Y;
X₂ is G;
X₃ is W or Y;
X₄ is G;
X₅ is V or I;
X₆ is T;
X₇ is Y, F or L;
X₈ is K;

(b) a protein domain which consists of an amino acid sequence that is at least 75% identical to the amino acid sequence of the protein domain of (a); or
(c) a protein domain which is a fragment of the protein domain of (a) or (b).

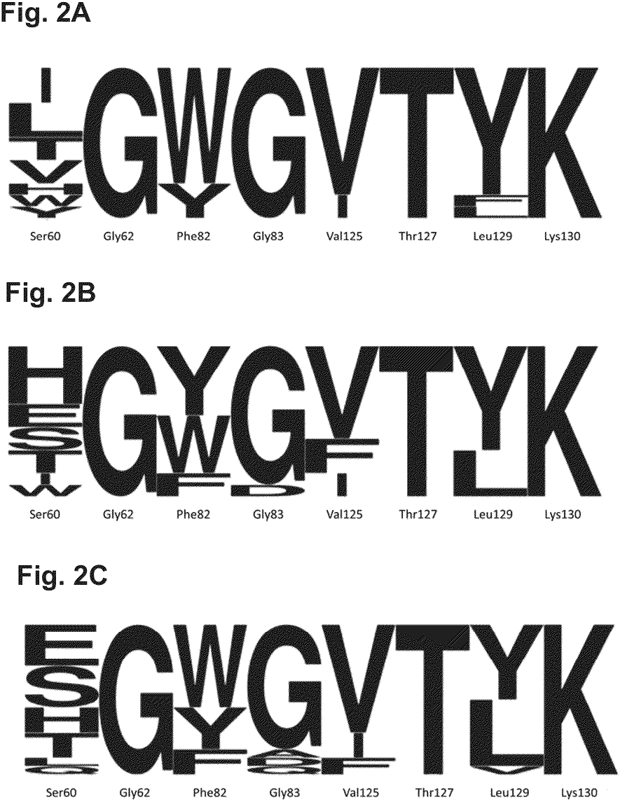

The compound according to this aspect reflects the mutations to B7-H6 that were found to result in a very strong increase in the affinity for NKp30 (KD < 40 nM, see FIG. 2A).

“Compound”, as used in the present disclosure, is not particularly limited and refers to a chemical entity of any chemical class, provided that it includes the protein domain as defined above. Thus, the compound can e.g. be an organic compound or a compound composed of an organic and an inorganic part, it can be a protein composed of a single amino acid chain, a protein composed of multiple amino acid chains that are either non-covalently or covalently associated, or a non-covalent complex including an inorganic component. The compound can consist of the amino acid sequence of the protein domain of (a), (b) or (c) alone or it can in addition include further amino acid(s) that may be covalently or non-covalently attached, or it can be associated with inorganic components. Preferably, the compound is a molecule.

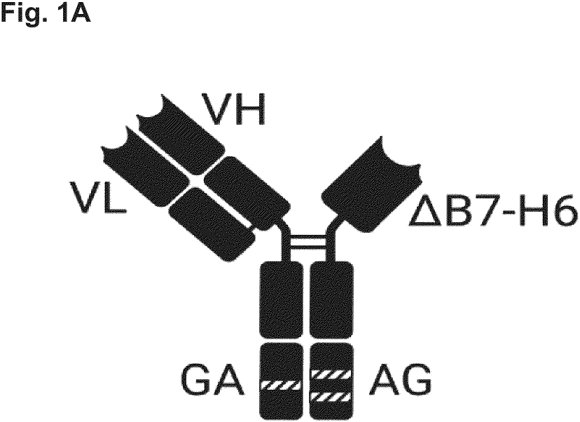

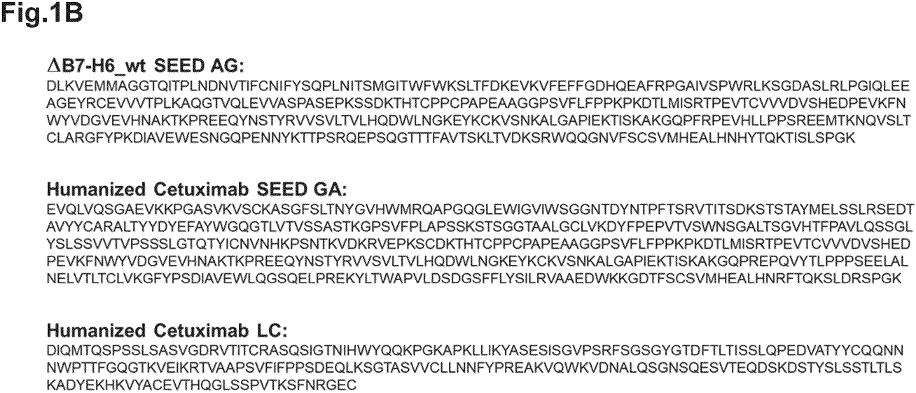

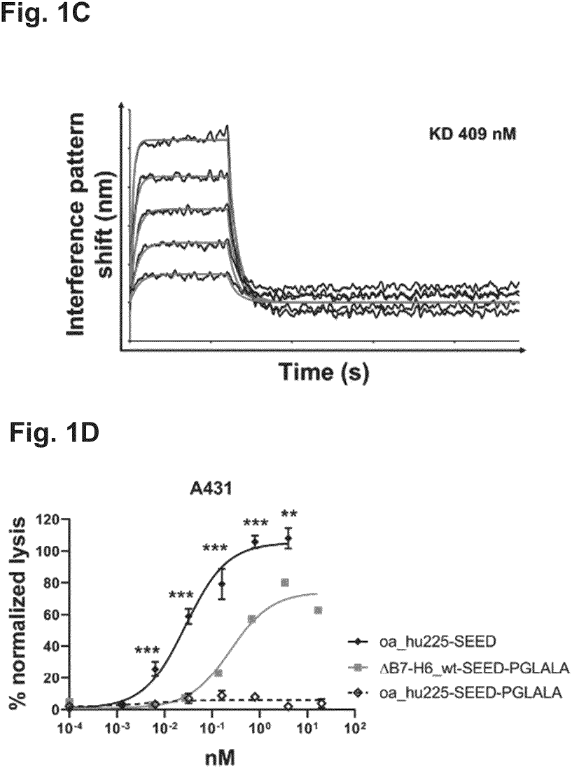

For example, the compound can be an immunoligand comprising a protein domain of (a), (b) or (c), linked covalently to an IgG1 antibody lacking one of its “arms”. The antibody may be prepared in the SEED format, resulting in an antibody with the structure shown in FIG. 1.

As the skilled person will understand, many other formats of the compound are possible, provided that the resulting compound does not interfere with the function of the protein domain of (a), (b) or (c), i.e. binding to NKp30 and activation of NK cells.

The term “protein domain” is used herein synonymously with the terms “polypeptide” and “chain of amino acids” (the term “polypeptide”, as used herein, does not imply any limitations with regard to the number of amino acids). The term “protein domain” designates a part of the compound that consists of a chain of amino acids and is typically linked by peptide bonds. In other words, the “protein domain” consists of a protein and is a part of the compound that is (covalently or non-covalently, but typically covalently) associated with the rest of the compound (unless the compound consists only of said protein domain and there are no other parts of the compound; in this case the compound is a protein consisting of the protein domain).

The rest of the compound to which the protein domain is linked may itself be a protein; in this case, the protein domain is just a part of the amino acid sequence of the complete compound. The term “protein domain” does not imply that the protein domain has a certain (e.g. compact) three-dimensional structure nor does it imply that the protein domain comprises a single structural fold.

The compound of the present disclosure can be prepared by standard methods of genetic engineering and recombinant protein technology known to the skilled person (see e.g. Green and Sambrook, “Molecular Cloning: A Laboratory Manual”, 2014; Coligan et al., “Current Protocols in Protein Science”, 1997). Exemplary methods are also described in the Examples section of the present disclosure.

In cases where the compound cannot be expressed in a single piece, individual parts can be prepared individually and later either covalently coupled, for example by a chemical reaction with appropriate reactive groups (e.g. linkage by maleimide chemistry) or by enzymatic linkage (e.g. transglutaminase-catalyzed linkage). For example, the protein domain of (a), (b) or (c) can be prepared by recombinant protein expression and subsequently linked to an antibody or antibody fragment, resulting in an immunoligand compound as described in the Examples section.

If the protein comprises components that are not biomolecules (such as a peptide mimetics or a small molecule), these components may be obtained e.g. by standard methods of synthetic organic chemistry.

If the present disclosure states that a certain sequence A “is at least x % identical” to another sequence B, this is synonymous to the statement that sequence A “has x % identity” to sequence B. The statement reflects a relationship between the two polypeptide sequences A and B determined by comparing the sequences. In general, identity refers to an exact amino acid to amino acid correspondence of the two polypeptide sequences, respectively, over the length of the sequences being compared. For sequences where there is not an exact correspondence, a percentage to which the two sequences are identical may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity of two or more sequences are well known in the art. Thus, for instance, programs available in the Wisconsin Sequence Analysis Package, version 9.1 (Devereux J et al., 1984), for example the programs BESTFIT and GAP, may be used to determine the % identity between two polynucleotides and the % identity between two polypeptide sequences. BESTFIT uses the “local homology” algorithm of Smith and Waterman (1981) and finds the best single region of similarity between two sequences. Other programs for determining identity sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson WR, 1990). Preferably, % identity according to the present disclosure is determined according to the BLAST family of programs (Altschul S F et al, 1990, Altschul S F et al, 1997, accessible through the home page of the NCBI at www.ncbi.nlm.nih.gov).

If the identity of a sequence A with a sequence B is determined in a situation where sequence B is a generalized amino acid sequence (such as the amino acid sequence of the protein domain of (a) above in which “X₁ is I, L, T, V, H, W or Y”), the position of X₁ is considered as identical between the two sequences if in an alignment the amino acid in sequence A aligning with X₁ is one of the amino acids falling under the definition of X₁ (i.e. if sequence A has at the position corresponding to X₁ in sequence B either I, L, T, V, H, W or Y).

At some occasions, the present disclosure states that a certain protein/amino acid sequence A is a “fragment” of another protein/amino acid sequence B. This means that, compared to protein/amino acid B, the protein/amino acid sequence A lacks one or more amino acids at the N-terminus and/or one or more amino acids at the C-terminus. Whether a protein/amino acid sequence lacks, compared to another protein/amino acid sequence, one or more amino acids at the N-terminus and/or one or more amino acids at the C-terminus can for example readily be determined upon forming a sequence alignment e.g. with the BLAST family of programs.

In the sequence of SEQ ID NO: 3,
X₁ is at the position corresponding to Ser60 in human full-length B7-H6 (SEQ ID NO: 1);
X₂ is at the position corresponding to Gly62 in human full-length B7-H6 (SEQ ID NO: 1);
X₃ is at the position corresponding to Phe82 in human full-length B7-H6 (SEQ ID NO: 1);
X₄ is at the position corresponding to Gly83 in human full-length B7-H6 (SEQ ID NO: 1);
X₅ is at the position corresponding to Val125 in human full-length B7-H6 (SEQ ID NO: 1);
X₆ is at the position corresponding to Thr127 in human full-length B7-H6 (SEQ ID NO: 1);
X₇ is at the position corresponding to Leu129 in human full-length B7-H6 (SEQ ID NO: 1);
X₈ is at the position corresponding to Lys130 in human full-length B7-H6 (SEQ ID NO: 1).

In some embodiments, in said protein domain of (a) X₁ is I, L, T or V.

In some embodiments, in said protein domain of (a) X₃ is W.

In some embodiments, in said protein domain of (a) X₅ is V.

In some embodiments, in said protein domain of (a) X₇ is Y.

According to another aspect, the present disclosure relates to a compound comprising

(a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3),

wherein
X₁ is H, E, S, T, I or W;
X₂ is G;
X₃ is Y, W or F;
X₄ is G or D;
X₅ is V, F or I;
X₆ is T;
X₇ is Y or L;
X₈ is K;

(b) a protein domain which consists of an amino acid sequence that is at least 75% identical to the amino acid sequence of the protein domain of (a); or
(c) a protein domain which is a fragment of the protein domain of (a) or (b).

The compound according to this aspect reflects the mutations to B7-H6 that were found to result in a strong increase in the affinity for NKp30 (KD = 40-120 nM, see FIG. 2B).

In some embodiments, in said protein domain of (a) X₁ is H, E, S or T.

In some embodiments, in said protein domain of (a) X₃ is Y or W.

In some embodiments, in said protein domain of (a) X₄ is G.

In some embodiments, in said protein domain of (a) X₅ is V.

In some embodiments, in said protein domain of (a) X₇ is Y.

According to another aspect, the present disclosure relates to a compound comprising

(a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3),

wherein
X₁ is E, S, H, T, L or Q;
X₂ is G;
X₃ is W, Y or F;
X₄ is G, A, D or Q;
X₅ is V, I or F;
X₆ is T;
X₇ is Y, L or V;
X₈ is K;

(b) a protein domain which consists of an amino acid sequence that is at least 75% identical to the amino acid sequence of the protein domain of (a); or
(c) a protein domain which is a fragment of the protein domain of (a) or (b).

The compound according to this aspect reflects the mutations to B7-H6 that were found to result in an increase in the affinity for NKp30 (with KD still > 120 nM, see FIG. 2C).

In some embodiments, in said protein domain of (a) X₁ is E, S, H or T.

In some embodiments, in said protein domain of (a) X₃ is W.

In some embodiments, in said protein domain of (a) X₄ is G.

In some embodiments, in said protein domain of (a) X₅ is V.

In some embodiments, in said protein domain of (a) X₇ is Y or L.

The following embodiments relate to any of the compounds defined above.

In some embodiments, said protein domain of (b) is capable of specifically binding to NKp30.

In some embodiments, the specific term “said protein domain of (b) is capable of specifically binding to NKp30” means that said protein domain of (b) is capable of binding to NKp30 with a specificity that is at least equal to the specificity with which human B7-H6 (SEQ ID NO: 1) binds to NKp30.

In some embodiments, said protein domain of (b) is, upon binding to NKp30 on NK cells, capable of activating NK cells.

Whether a protein domain is, upon binding to NKp30 on NK cells, capable of activating NK cells can be determined as described in Example 1, section “NK cell activation assay”. If this activation occurs upon binding to NKp30 on NK cells can for example be determined by carrying out a control experiment with NK cells in which NKp30 has been blocked by a competitor molecule that binds to NKp30 such that the protein domain cannot get access to NKp30 on the NK cells.

Preferably, binding to NKp30 and activation of NK cells is assessed with said protein domain of (b) in the context of the complete compound.

In some embodiments, activation of NK cells is measured in an assay in which 20,000 A431 cells/well are seeded in 96-well V-bottom microtiter plates and incubated for 3 h prior addition of 100,000 NK cells/well (E:T ratio of 5:1), that had previously been treated with 100 U/ml recombinant human interleukin-2 overnight, said compound to be tested is then added at a final concentration of 85 nM, the cells are incubated for 24 h at 37° C., washed two times with PBS + 1% BSA, incubated with dead cell stain, anti-human CD56 and anti-human CD69 for 1 h on ice, washed, followed by measurement of activated NK cells based on a gating strategy involving Side scatter (SSC) vs. forward scatter (FCS) for identification of single NK cells, followed by dead cell staining and CD56 staining versus an unrelated channel for the identification of living CD56⁺ NK cells followed by gating on activated CD56⁺ CD69⁺ NK cells, wherein a compound to be tested is considered to activate NK cells if the number of activated NK cells is increased in the presence of said compound to be tested.

In some embodiments, said protein domain of (b) consists of an amino acid sequence that is at least 80% identical to the amino acid sequence of the protein domain of (a).

In some embodiments, said protein domain of (b) consists of an amino acid sequence that is at least 85% identical to the amino acid sequence of the protein domain of (a).

In some embodiments, said protein domain of (b) consists of an amino acid sequence that is at least 90% identical to the amino acid sequence of the protein domain of (a).

In some embodiments, said protein domain of (b) consists of an amino acid sequence that is at least 95% identical to the amino acid sequence of the protein domain of (a).

In some embodiments, said protein domain of (b) consists of an amino acid sequence that is at least 98% identical to the amino acid sequence of the protein domain of (a).

In some embodiments, said protein domain of (b) consists of an amino acid sequence that is at least 99% identical to the amino acid sequence of the protein domain of (a).

In some embodiments, said protein domain of (b) has at the positions corresponding to the positions of X₁, X₂, X₃, X₄, X₅, X₆, X₇ and X₈ of SEQ ID NO: 3 the same residues as defined for the protein domain of (a).

If the present disclosure refers to a position in a protein/amino acid sequence A “corresponding to” the position of a certain residue R in a protein/amino acid sequence B, this designates the amino acid in sequence A (or the gap) that in an alignment of the two proteins/amino acid sequences aligns with residue R in sequence B.

In some embodiments, in said protein domain of (b) the amino acids X₁, X₂, X₃, X₄, X₅, X₆, X₇ and X₈ are as defined in (a).

In some embodiments, in an alignment of said protein domain of (b) with SEQ ID NO: 3, the amino acids of the protein domain of (b) corresponding to X₁, X₂, X₃, X₄, X₅, X₆, X₇ and X₈ in SEQ ID NO: 3 are as defined in (a).

In some embodiments, said protein domain of (b) has
at the amino acid position of the protein domain of (b) that corresponds to the position of X₁ in SEQ ID NO: 3 (in an alignment of the protein domain of (b) with SEQ ID NO: 3): an amino acid that is identical to the amino acid/one of the amino acids in the definition of X₁ in the protein domain of (a);
at the amino acid position of the protein domain of (b) that corresponds to the position of X₂ in SEQ ID NO: 3 (in an alignment of the protein domain of (b) with SEQ ID NO: 3): an amino acid that is identical to the amino acid/one of the amino acids in the definition of X₂ in the protein domain of (a);
at the amino acid position of the protein domain of (b) that corresponds to the position of X₃ in SEQ ID NO: 3 (in an alignment of the protein domain of (b) with SEQ ID NO: 3): an amino acid that is identical to the amino acid/one of the amino acids in the definition of X₃ in the protein domain of (a);
at the amino acid position of the protein domain of (b) that corresponds to the position of X₄ in SEQ ID NO: 3 (in an alignment of the protein domain of (b) with SEQ ID NO: 3): an amino acid that is identical to the amino acid/one of the amino acids in the definition of X₄ in the protein domain of (a);
at the amino acid position of the protein domain of (b) that corresponds to the position of X₅ in SEQ ID NO: 3 (in an alignment of the protein domain of (b) with SEQ ID NO: 3): an amino acid that is identical to the amino acid/one of the amino acids in the definition of X₅ in the protein domain of (a);
at the amino acid position of the protein domain of (b) that corresponds to the position of X₆ in SEQ ID NO: 3 (in an alignment of the protein domain of (b) with SEQ ID NO: 3): an amino acid that is identical to the amino acid/one of the amino acids in the definition of X₆ in the protein domain of (a);
at the amino acid position of the protein domain of (b) that corresponds to the position of X₇ in SEQ ID NO: 3 (in an alignment of the protein domain of (b) with SEQ ID NO: 3): an amino acid that is identical to the amino acid/one of the amino acids in the definition of X₇ in the protein domain of (a);
at the amino acid position of the protein domain of (b) that corresponds to the position of X₈ in SEQ ID NO: 3 (in an alignment of the protein domain of (b) with SEQ ID NO: 3): an amino acid that is identical to the amino acid/one of the amino acids in the definition of X₈ in the protein domain of (a).

In some embodiments, the amino acids X₁, X₂, X₃, X₄, X₅, X₆, X₇ and X₈ of the protein domain of (a) are not replaced by another amino acid or deleted in said protein domain of (b) compared to said protein domain of (a).

According to another aspect, the present disclosure relates to a compound comprising

(a) a protein domain which consists of the amino acid sequence of any one of the following sequences:

Table of Sequences

| | |
|---|---|
| SEQ ID NO | Amino acid sequence |
| SEQ ID NO: 4 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 5 | DLKVEMMAGGTQITPLNDNVTIFCNIFYSQPLNITWMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 6 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVFVTPYKAQGTVQLEVVASPAS |
| SEQ ID NO: 7 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 8 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 9 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 10 | DLKVEMMAGGTQITPLNDNVTIFCNIFYSQPLNITHMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 11 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVIVTPLKAQGTVQLEVVASPAS |
| SEQ ID NO: 12 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 13 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPYKAQGTVQLEVVASPAS |
| SEQ ID NO: 14 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVIVTPFKAQGTVQLEVVASPAS |
| SEQ ID NO: 15 | DLKVEMMAGGTQITPLNDNVTIFCNIFYSQPLNITEMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 17 | DLKVEMMAGGTQITPLNDNVTIFCNIFYSQPLNITTMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 18 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 19 | DLKVEMMAGGTQITPLNDNVTIFCNIFYSQPLNITLMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 20 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPLKAQGTVQLEVVASPAS |
| SEQ ID NO: 21 | DLKVEMMAGGTQITPLNDNVTIFCNIFYSQPLNITYMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 22 | DLKVEMMAGGTQITPLNDNVTIFCNIFYSQPLNITQMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 23 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVFVTPLKAQGTVQLEVVASPAS |
| SEQ ID NO: 24 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 25 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 26 | DLKVEMMAGGTQITPLNDNVTIFCNIFYSQPLNITEMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 27 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 28 | DLKVEMMAGGTQITPLNDNVTIFCNIFYSQPLNITWMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 29 | DLKVEMMAGGTQITPLNDNVTIFCNIFYSQPLNITTMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 30 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 31 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 32 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 33 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVIVTPLKAQGTVQLEVVASPAS |
| SEQ ID NO: 34 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 35 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVVVTPVKAQGTVQLEVVASPAS |
| SEQ ID NO: 36 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 37 | DLKVEMMAGGTQITPLNDNVTIFCNIFYSQPLNITSMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 38 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 39 | DLKVEMMAGGTQITPLNDNVTIFCNIFYSQPLNITHMGITW FWKSLTFDKEVKVFEFYADHQEAIRPGAIVSPWRLKSGDAS LRLPGIQLEEAGEYRCEVVVTPYKAQGTVQLEVVASPAS |
| SEQ ID NO: 40 | DLKVEMMAGGTQITPLNDNVTIFCNIFYSQPLNITSMGITW LWKSLTFDKEVKVFEFWGDHQEAFRPGAIVSPWRLKSGDAS LRLPGIQLEEAGEYRCEVFVTPYKAQGTVQLEVVASPAS |
| SEQ ID NO: 41 | DLKVEMMAGGTQITPLNDNVTIFCNIFYFQPLNITEMGITW FWKSLTFDKEVKVFEFFGDHQEAFRPGAIVSPWRLKSGDAS LRLPGIQLEEAGEYRCEVIVTPLKAQGTVQLEVVASPAS |
| SEQ ID NO: 42 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVIVTPYKAQGTVQLEVVTSPAS |
| SEQ ID NO: 43 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCGVVVTPVKAQGTVQLEVVASPAS |
| SEQ ID NO: 44 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVVVTPLKAQGTAQLEVVASPAS |
| SEQ ID NO: 45 | DLKVEMMAGGTQITPLNDNVTIFCNIFYSQPLNITLMGITW FWKSLTFDKEVKVFEFWGDHQEAFRPGAIVSPWGLKSGDAS LRLPGIQLEEAGEYRCEVVVTPLKAQGTVQLEVVASPAS |
| SEQ ID NO: 46 | DLKVEMMAGGTQITPLNDNVTIFCNIFYSQPLNITTMGITW FWKSLTFDKEVKVFEFWQDHQEAFRPGAIVSPWRLKSGDAS LRLPGIQLEEAGEYRCEVVVTPLKAQGTVQLEVVASPAS |
| SEQ ID NO: 47 | DLKVEMMAGGTQITPLNDNVTIFCNIFYSQPLNITHMGITW LWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 48 | DLKVEMMAGGTQITPLNDNVTIFCNIFYSQPLNITSMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 49 | DLKVEMMAGGTQITPLNDNVTIFCNIFYSQPLNITTMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 50 | DLKVEMMAGGTQITPLNDNVTIFCNIFYSQPLNITEMGITW FWKSLTFDKEVKVFEFYGDHQEAFXPGAIVSPWRLKSGDAS LXLPGIQLEEAGEYRCEVVVTPLKAQGTVQLEVVASPAS |

(b) a protein domain which consists of an amino acid sequence that is identical to at least one sequence listed under (a) with the only difference that in up to 30 occasions an amino acid is individually added, replaced by another amino acid or deleted in the amino acid sequence of the protein domain of (b) compared to said at least one sequence listed under (a).

(c) a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 30 occasions an amino acid is individually added, replaced by another amino acid or deleted in the amino acid sequence of the protein domain of (c) compared to said at least one sequence listed under (a).

If the present applications states that “in up to 30 occasions” an amino acid is “individually replaced another amino acid” (or is “individually deleted”; or is “individually added”) in an amino acid sequence A compared to an amino acid sequence B, this means that in an alignment of sequences A and B at up to 30 amino acid positions of the alignment sequence A has a different amino acid than sequence B (or at up to 30 amino acid positions of the alignment sequence A has no amino acid where sequence B has an amino acid; or at up to 30 amino acid positions of the alignment sequence A has an amino acid where sequence B has no amino acid).

In some embodiments, said protein domain of (a) consists of the amino acid sequence of any one of the sequences listed in the “Table of Sequences” above.

In some embodiments, said protein domain of (b) consists of an amino acid sequence that is identical to at least one sequence listed under (a) with the only difference that in up to 30 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 24 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 18 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 12 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 6 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 5 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 4 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 3 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 2 occasions an amino acid is individually added, replaced by another amino acid or deleted in the amino acid sequence of the protein domain of (b) compared to said at least one sequence listed under (a).

In some embodiments, said protein domain of (b) consists of an amino acid sequence that is identical to at least one sequence listed under (a) with the only difference that in up to 1 occasion an amino acid is individually added, replaced by another amino acid or deleted in the amino acid sequence of the protein domain of (b) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 24 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 18 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 12 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 6 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 5 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 4 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 3 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 2 occasions an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 1 occasion an amino acid is individually added, replaced by another amino acid or deleted in the amino acid sequence of the protein domain of (c) compared to said at least one sequence listed under (a).

In some embodiments, said protein domain of (c) is a protein domain which consists of an amino acid sequence that is a fragment of the amino acid sequence of any one of the sequences listed in (a).

In some embodiments, an amino acid is added if said amino acid is present in the amino acid sequence of said protein domain of (b), but said amino acid is absent at the corresponding position of said at least one sequence listed under (a).

In some embodiments, an amino acid is replaced by another amino acid if said amino acid is present in said at least one sequence listed under (a), but absent at the corresponding position in the amino acid sequence of said protein domain of (b), and instead another amino acid is present in the amino acid sequence of said protein domain of (b) at the respective position.

In some embodiments, said replacement is a conservative amino acid replacement.

As used herein, a “conservative amino acid replacement” refers to the replacement of an amino acid by another, biologically similar amino acid. Conservative replacements are not likely to change the shape or characteristics of a protein/amino acid sequence. Examples of conservative replacements include the replacement of one hydrophobic residue such as isoleucine, valine, leucine or methionine for another, or the substitution of one polar residue for another, such as the substitution of arginine for lysine, glutamic for aspartic acid, or glutamine for asparagine.

In some embodiments, an amino acid is deleted if said amino acid is present in said at least one sequence listed under (a), but absent at the corresponding position of the amino acid sequence of said protein domain of (b).

In some embodiments, none of the amino acids shown in bold in said at least one sequence listed under (a) is replaced or deleted in the amino acid sequence of said protein domain of (b).

In some embodiments, all amino acids depicted in bold in said at least one sequence listed under (a) are conserved in the amino acid sequence of said protein domain of (b).

In some embodiments, said protein domain of (c) is capable of specifically binding to NKp30.

In some embodiments, said protein domain of (c) is, upon binding to NKp30 on NK cells, capable of activating NK cell.

In some embodiments, said protein domain of (c) consists of at least 75 amino acids.

In some embodiments, said protein domain of (c) consists of at least 80 amino acids.

In some embodiments, said protein domain of (c) consists of at least 90 amino acids.

In some embodiments, said protein domain of (c) consists of at least 100 amino acids.

In some embodiments, said protein domain of (c) consists of at least 110 amino acids.

In some embodiments, said protein domain of (c) consists of at least 120 amino acids.

In some embodiments, said protein domain of (c) consists of an amino acid sequence corresponding to the amino acid sequence from amino acid 55 to amino acid 135 of SEQ ID NO: 1.

In some embodiments, said protein domain of (c) consists of an amino acid sequence corresponding to the amino acid sequence from amino acid 50 to amino acid 140 of SEQ ID NO: 1.

In some embodiments, said protein domain of (c) is a fragment of the protein domain of (a).

In some embodiments, said protein domain of (a), (b) or (c) does not comprise the amino acid sequence of SEQ ID NO: 2.

In some embodiments, said compound does not comprise the amino acid sequence of SEQ ID NO: 2.

In some embodiments, in said protein domain of (a) at least one of the following does not apply:
X₁ is S;
X₃ is F;
X₄ is G;
X₅ is V;
X₇ is L.

In some embodiments, in said protein domain of (a) at least two of the following do not apply:
X₁ is S
X₃ is F;
X₄ is G;
X₅ is V;
X₇ is L.

In some embodiments, wherein in said protein domain of (a) at least three of the following do not apply:
X₁ is S;
X₃ is F;
X₄ is G;
X₅ is V;
X₇ is L.

In some embodiments, said protein domain of (a) consists of an amino acid sequence selected from the group consisting of SEQ ID NO: 4, 6, 8, 9, 10, 11, 12, 13, 14, 15, 17, 18, 19, 20, 21, 22, 24, 25, 26, 27, 28, 29, 30, 32, 33, 34, 36, 38, 42, 45 and 48. (As can be seen e.g. from Table 4 in Example 4, compounds with such a protein domain show a KD improvement factor ≥ 2 compared to ΔB7-H6_wt.)

In some embodiments, said protein domain of (a) consists of an amino acid sequence selected from the group consisting of SEQ ID NO: 4, 8, 9, 11, 12, 14, 17, 18, 19, 20, 21, 24, 27, 28, 32 and 34. (As can be seen e.g. from Table 4 in Example 4, compounds with such a protein domain show a KD improvement factor ≥ 5 compared to ΔB7-H6_wt.)

In some embodiments, said protein domain of (a) consists of an amino acid sequence selected from the group consisting of SEQ ID NO: 4, 8, 9, 14, 17, 18, 19, 21, 24, 27 and 32. (As can be seen e.g. from Table 4 in Example 4, compounds with such a protein domain show a KD improvement factor ≥ 10 compared to ΔB7-H6_wt.)

In some embodiments, said protein domain of (a) consists of an amino acid sequence selected from the group consisting of SEQ ID NO: 8, 18, 21, 24, 27 and 32. (As can be seen e.g. from Table 4 in Example 4, compounds with such a protein domain show a KD improvement factor ≥ 15 compared to ΔB7-H6_wt.)

In some embodiments, said protein domain of (a) consists of an amino acid sequence selected from the group consisting of SEQ ID NO: 18, 21, 24 and 27. (As can be seen e.g. from Table 4 in Example 4, compounds with such a protein domain show a KD improvement factor ≥ 20 compared to ΔB7-H6_wt.)

In some embodiments, said protein domain of (a) consists of an amino acid sequence selected from the group consisting of SEQ ID NO: 4, 8, 9, 10, 11, 12, 13, 14, 17, 18, 19, 21, 24, 26, 27, 28, 29, 30, 32 and 34. (As can be seen e.g. from Table 6 in Example 5, compounds with such a protein domain show an EC50 improvement factor ≥ 10 compared to ΔB7-H6_wt.)

In some embodiments, said protein domain of (a) consists of an amino acid sequence selected from the group consisting of SEQ ID NO: 9, 11, 17, 18, 19, 21, 24, 27, 28, 29, 30, 32 and 34. (As can be seen e.g. from Table 6 in Example 5, compounds with such a protein domain show an EC50 improvement factor ≥ 20 compared to ΔB7-H6_wt.)

In some embodiments, said protein domain of (a) consists of an amino acid sequence selected from the group consisting of SEQ ID NO: 17, 18, 21, 27, 28, 30, 32 and 34. (As can be seen e.g. from Table 6 in Example 5, compounds with such a protein domain show an EC50 improvement factor ≥ 40 compared to ΔB7-H6_wt.)

In some embodiments, said protein domain of (a) consists of an amino acid sequence selected from the group consisting of SEQ ID NO: 18 and 32. (As can be seen e.g. from Table 6 in Example 5, compounds with such a protein domain show an EC50 improvement factor ≥ 100 compared to ΔB7-H6_wt.)

In some embodiments, said protein domain of (a) consists of an amino acid sequence selected from the group consisting of SEQ ID NO: 4, 6, 8, 9, 10, 11, 12, 13, 14, 15, 17, 18, 19, 21, 22, 24, 25, 26, 27, 28, 29, 30, 31, 33, 34, 36, 38, 42, 45, 46, 48, 49 and 50. (As can be seen e.g. from Table 6 in Example 5, compounds with such a protein domain show a max. killing improvement factor ≥ 1.3 compared to ΔB7-H6_wt.)

In some embodiments, said protein domain of (a) consists of an amino acid sequence selected from the group consisting of SEQ ID NO: 4, 6, 9, 10, 11, 12, 13, 15, 19, 21, 30, 31, 36, 42, 45, 48 and 49. (As can be seen e.g. from Table 6 in Example 5, compounds with such a protein domain show a max. killing improvement factor ≥ 1.5 compared to ΔB7-H6_wt.)

In some embodiments, said protein domain of (a) consists of an amino acid sequence selected from the group consisting of SEQ ID NO: 13, 19, 30 and 48. (As can be seen e.g. from Table 6 in Example 5, compounds with such a protein domain show a max. killing improvement factor ≥ 1.7 compared to ΔB7-H6_wt.)

In some embodiments, said protein domain of (a) consists of an amino acid sequence selected from the group consisting of SEQ ID NO: 17, 18, 19, 21, 24, 27, 28, 29, 30, 32 and 34. (As can be seen e.g. from Example 6, compounds with such a protein domain show an IFNγ release improvement factor ≥ 1 compared to ΔB7-H6_wt.)

In some embodiments, said protein domain of (a) consists of an amino acid sequence selected from the group consisting of SEQ ID NO: 18, 19, 21 and 27. (As can be seen e.g. from Example 6, compounds with such a protein domain show an IFNγ release improvement factor > 3 compared to ΔB7-H6_wt.)

In some embodiments, said protein domain of (a) consists of an amino acid sequence selected from the group consisting of SEQ ID NO: 17, 18, 19, 21, 24, 27, 28, 29, 30, 32 and 34. (As can be seen e.g. from Example 6, compounds with such a protein domain show a TNFα release improvement factor > 1 compared to ΔB7-H6_wt.)

In some embodiments, said protein domain of (a) consists of an amino acid sequence selected from the group consisting of SEQ ID NO: 18, 19, 21, 24, 27, 28, 30, 32 and 34. (As can be seen e.g. from Example 6, compounds with such a protein domain show a TNFα release improvement factor > 8 compared to ΔB7-H6_wt.)

In some embodiments, said protein domain of (a) consists of an amino acid sequence selected from the group consisting of SEQ ID NO: 18, 21, 27, 30 and 34. (As can be seen e.g. from Example 6, compounds with such a protein domain show a TNFα release improvement factor > 10 compared to ΔB7-H6_wt.)

In some embodiments, said compound comprises or is a protein.

By stating that the compound “comprises” a protein, the present disclosure designates that the compound includes a part within its chemical structure that is a protein. A compound that comprises a protein may or may not comprise a part that is not a protein.

In some embodiments, said compound is a protein.

By stating that the compound “is” a protein, the present disclosure designates that the compound consists only of protein and does not comprise a part that is not a protein.

In some embodiments, said compound comprises said protein domain of (a) or (b).

In some embodiments, said compound comprises said protein domain of (a) or (c).

In some embodiments, said compound consists of said protein domain of (a), (b) or (c).

In some embodiments, said compound consists of said protein domain of (a) or (b).

In some embodiments, said compound consists of said protein domain of (a) or (c).

In some embodiments, said compound further comprises a targeting moiety.

As used herein, the term “targeting moiety” refers to a moiety (i.e. a molecular group or chemical structure) that is (typically covalently) associated with said compound and that binds a target site, wherein said binding allows to recruit the compound to said target site. The target site will typically be a biological molecule or a certain part of a biological molecule. An example of a targeting moiety is an antigen-binding antibody fragment that is covalently linked to an affinity-maturated B7-H6 IgV-like domain to form a compound according to the present disclosure, wherein the antigen-binding fragment binds to a certain receptor present at the surface of a certain cell type (its antigen), and wherein binding of the antigen-binding fragment to this receptor results in recruitment of the compound to this cell type.

Non-targeted drugs typically reach their site of action by whole-body distribution and passive diffusion. In contrast, targeted compounds are not distributed evenly across the whole body. Due to the interaction of targeting moiety with its target molecule, a compound including a targeting moiety is concentrated preferentially at its site target site. Therefore, e.g. therapeutic compounds with a targeting moiety require lower dosages to be therapeutically effective, thus improving the therapeutic window.

In some embodiments, said compound comprises
said protein domain of (a), (b) or (c) and
a targeting moiety.

In some embodiments, said compound consists of
said protein domain of (a), (b) or (c) and
a targeting moiety.

In some embodiments, said compound comprises
said protein domain of (a) and
a targeting moiety.

In some embodiments, said compound consists of
said protein domain of (a) and
a targeting moiety.

In some embodiments, all components of said compound are covalently linked.

In some embodiments, said targeting moiety is a molecular group that specifically binds to a target molecule or fragment thereof.

In some embodiments, said target molecule is a receptor at the surface of a cell.

In some embodiments, said target molecule is an antigen that is present on the surface of a target cell.

In some embodiments, said targeting moiety is capable of specifically binding to a target molecule (preferably an antigen) that is present on the surface of a target cell.

As used herein, a “target molecule that is present on the surface of a target cell” is a molecule that is present on the surface of the target cell in such a manner that it is accessible from the extracellular environment (i.e. e.g. an antibody can bind to it from the extracellular environment). For example, CD8 is a transmembrane protein of cytotoxic T cells, and its extracellular domain is accessible for antibodies directed against the extracellular domain of CD8 from the extracellular environment. Thus, in the sense of the present disclosure, CD8 is a target molecule that is present on the surface of cytotoxic T cells.

A targeting moiety that “binds” a target molecule of interest is a targeting moiety that is capable of binding that target molecule with sufficient affinity such that the targeting moiety is useful in targeting the compound to a cell expressing the target molecule.

If the present disclosure refers to a first molecule/molecular group (e.g. an antibody/antibody component) “specifically binding″/that “specifically binds” to a second molecule/molecular group (e.g. an antigen of interest), this means that the first molecule/molecular group (in this example the antibody) binds to said second molecule/molecular group (in this example the antigen of interest) with an affinity that is at least ten-fold greater than its affinity for other molecules/molecular groups, in particular other molecule/molecular group in the human body (in this example at least ten-fold greater than its affinity for binding to non-specific antigens (e.g., BSA, casein) other than said antigen of interest (or closely related antigens)). In a preferred embodiment, a first molecule/molecular group (e.g. an antibody/antibody component) that “specifically binds” to a second molecule/molecular group (e.g. an antigen of interest) binds to said antigen with an affinity that is at least 100-fold greater than its affinity for other molecules/molecular groups, in particular other molecule/molecular group in the human body (in this example at least 100-fold greater than its affinity for binding to non-specific antigens other than said antigen of interest (or closely related antigens)). Typically said binding will be determined under physiological conditions. A first molecule/molecular group that “specifically binds” to a second molecule/molecular group may bind to that second molecule/molecular group with an affinity of at least about 1×10⁷ M⁻¹. An antibody/antibody component that “specifically binds” to an antigen of interest may bind to that antigen with an affinity of at least about 1×10⁷ M⁻¹.

In some embodiments, said targeting moiety comprises a protein, a peptide, a peptide mimetic, a nucleic acid, an oligonucleotide or a small molecule.

In some embodiments, said targeting moiety is a protein, a peptide, a peptide mimetic, a nucleic acid, an oligonucleotide or a small molecule.

In some embodiments, said targeting moiety comprises a protein.

In some embodiments, said targeting moiety is a protein.

In some embodiments, said targeting moiety comprises or is a protein which is a protein ligand that specifically binds to a receptor at the surface of a cell.

In some embodiments, said targeting moiety comprises or is a protein which is an antibody or an antigen-binding fragment thereof.

In some embodiments, said targeting moiety comprises or is a protein which comprises at least 30 amino acids.

In some embodiments, said targeting moiety comprises or is a peptide which consists of 2 to 30 amino acids.

In some embodiments, said targeting moiety comprises or is a peptide which consists of 10 to 30 amino acids.

In some embodiments, said targeting moiety comprises a peptide.

In some embodiments, said targeting moiety is a peptide.

In some embodiments, said targeting moiety comprises a peptide mimetic.

In some embodiments, said targeting moiety is a peptide mimetic.

As used herein, the term “peptide mimetic” refers to a peptide-like chain which is designed to mimic a peptide. An example of a peptide mimetic is a D-peptide mimetic containing a D-amino acid, but is not limited thereto.

In some embodiments, said targeting moiety comprises or is a nucleic acid which is a DNA or an RNA.

In some embodiments, said targeting moiety comprises a nucleic acid.

In some embodiments, said targeting moiety is a nucleic acid.

In some embodiments, said targeting moiety comprises an oligonucleotide.

In some embodiments, said targeting moiety is an oligonucleotide.

In some embodiments, said targeting moiety comprises or is a small molecule with a molecular weight < 1000 Da.

In some embodiments, said targeting moiety comprises a small molecule.

In some embodiments, said targeting moiety is a small molecule.

In some embodiments, said targeting moiety has a molecular weight of at least 100 Da.

In some embodiments, said targeting moiety has a molecular weight of at least 500 Da.

In some embodiments, said targeting moiety has a molecular weight of at least 1 000 Da.

In some embodiments, said targeting moiety has a molecular weight of at least 2 000 Da.

In some embodiments, said targeting moiety has a molecular weight of at least 10 kDa.

In some embodiments, said targeting moiety has a molecular weight of at least 50 kDa.

In some embodiments, said targeting moiety has a molecular weight of at least 100 kDa.

In some embodiments, said targeting moiety has a molecular weight of up to 1 000 Da.

In some embodiments, said targeting moiety has a molecular weight of up to 2 000 Da.

In some embodiments, said targeting moiety has a molecular weight of up to 10 kDa.

In some embodiments, said targeting moiety has a molecular weight of up to 50 kDa.

In some embodiments, said targeting moiety has a molecular weight of up to 200 kDa.

In some embodiments, said targeting moiety has a molecular weight of up to 1 MDa.

In some embodiments, said targeting moiety has a molecular weight of up to 5 MDa.

In some embodiments, said targeting moiety has a molecular weight of up to 10 MDa.

In some embodiments, said targeting moiety is capable of specifically binding to a tumor-associated antigen or to an immune cell antigen.

In some embodiments, said targeting moiety is capable of specifically binding to a tumor-associated antigen.

In some embodiments, said targeting moiety comprises an antibody or antigen-binding fragment of an antibody.

In some embodiments, said targeting moiety is an antibody or antigen-binding fragment of an antibody.

The term “antibody”, as used herein, refers to a polypeptide that includes immunoglobulin sequence elements sufficient to confer specific binding to a particular target antigen. The term “antibody” can encompass intact antibodies and antigen-binding fragments of intact antibodies (i.e. fragments of an intact antibody that are still capable of binding the same antigen to which the corresponding intact antibody binds). In some embodiments, the term also includes molecules in which an intact antibody or antigen-binding fragment of an intact antibody is covalently linked to one or more further intact antibodies and/or one or more further antigen-binding fragments of antibodies and/or another molecular structure. Thus, the antibody is an immunoglobulin molecule that recognizes and specifically binds to a target (the antigen, see below), through at least one antigen-binding site within the variable region of the immunoglobulin molecule.

As used herein, an “intact” antibody refers to an antibody that includes the complete, full-length sequence of an antibody of the respective antibody class. Thus, an intact antibody includes the antigen-binding region(s) (i.e. the complete VL and VH domains), as well as complete light and heavy chain constant domains, as appropriate for the antibody class, wherein the antibody domains remain associated through at least one non-covalent interaction. The constant domains may be native sequence constant domains (e.g., human native sequence constant domains) or amino acid sequence variants thereof.

As used herein, a “fragment” of an antibody is a portion of an intact antibody. An “antigen-binding fragment” of an (intact) antibody is a portion of said antibody that binds the same antigen as the intact antibody. Typically, this means that the fragment comprises the same antigen-binding region as the intact antibody. Examples of antibody fragments include, but are not limited to Fab, Fab′, F(ab′)2, and Fv fragments and single chain Fv (scFv) antibodies. In some embodiments, the term “fragment” of an antibody also encompasses bi- or multivalent antibody constructs generated by joining two or more of the aforementioned antibody fragments together.

As used herein, “antigen” refers to a substance that can specifically bind to the variable region of an antibody. An antigen may e.g. be a protein, polypeptide, peptide, carbohydrate, polynucleotide, lipid, or a combination of the foregoing.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of the antibody. There are at least two techniques for determining CDRs: (1) an approach based on cross-species sequence variability (Sequences of Proteins of Immunological Interest, 5th ed. (1991), editors Kabat et al., National Institutes of Health (Bethesda, USA)); and (2) an approach based on crystallographic studies of antigen-antibody complexes (Al-Lazikani et al., J. Molec. Biol. (1997), vol. 273, p. 927-948)). In addition, combinations of these two approaches are sometimes used in the art to determine CDRs.

The terms “epitope” or “antigenic determinant” are used interchangeably herein and refer to the portion of an antigen that is recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

With respect to the type and source, the antibody of the present disclosure is not particularly limited, as long as it contains at least one antigen-binding site and shows binding to its target antigen. Standard techniques of antibody design and preparation are known to a skilled person (see e.g. Antibodies: A Laboratory Manual, 2nd edition (2014), editor Greenfield, Cold Spring Harbor Laboratory Press (U.S.); Antibody Engineering - Methods and Protocols, 2nd edition (2010), editors Nevoltris and Chames, publisher Springer (Germany); Handbook of Therapeutic Antibodies (2014), editors Dübel and Reichert, publisher Wiley-VCH Verlag GmbH & Co. KGaA (Germany); Harper, Methods in Molecular Biology (2013), vol. 1045, p. 41-49).

In some embodiments, said targeting moiety is an antibody.

In some embodiments, said antibody is an intact antibody.

In some embodiments, said antibody is a monoclonal antibody or a polyclonal antibody.

A “monoclonal” antibody”, as used herein, means an antibody arising from a nearly homogeneous antibody population. More particularly, the individual antibodies of a population are identical except for a few possible naturally-occurring mutations which can be found in minimal proportions. In other words, a monoclonal antibody consists of a homogeneous antibody arising from the growth of a single cell clone and is generally characterized by heavy chains of one and only one class and subclass, and light chains of only one type. Monoclonal antibodies are directed against a single antigen. In addition, in contrast with preparations of polyclonal antibodies which typically include various antibodies directed against various epitopes, each monoclonal antibody is directed against a single epitope of the antigen. Monoclonal antibodies are typically produced by a single clone of B lymphocytes (“B cells”). Monoclonal antibodies may be obtained using a variety of techniques known to those skilled in the art, including standard hybridoma technology (see e.g. Köhler and Milstein, Eur. J. Immunol. (1976), vol. 5, p. 511-519; Antibodies: A Laboratory Manual, 2nd edition (2014), editor Greenfield, Cold Spring Harbor Laboratory Press (USA); Immunobiology, 5th ed. (2001), editors Janeway et al., Garland Publishing (USA)) and e.g. expression from a eukaryotic host cell transfected with a DNA molecule coding for the homogeneous antibody or from a prokaryotic host cell transfected with a DNA molecule coding for the homogeneous antibody.

As used herein, “polyclonal” antibody refers to a heterogeneous population of antibodies, typically obtained by purification from the sera of immunized animals by standard techniques known to a skilled person (see e.g. Antibodies: A Laboratory Manual, 2nd edition (2014), editor Greenfield, Cold Spring Harbor Laboratory Press (USA)).

In some embodiments, said antibody is a monoclonal antibody.

In some embodiments, said antibody is a monospecific antibody or a bispecific antibody.

A “monospecific antibody”, as used herein, is an antibody that is capable of binding only to one antigen.

The term “bispecific antibody”, as used in the present disclosure, refers to an antibody that is capable of specifically binding to two different epitopes at the same time. The epitopes can be from the same antigen or from two different antigens. Preferably, the epitopes are from two different antigens. Typically, a bispecific antibody has two antigen-binding sites, wherein e.g. each of the two pairs of heavy chain and light chain (HC/LC) is specifically binding to a different antigen, i.e. the first heavy and the first light chain are specifically binding together to a first antigen, and, the second heavy and the second light chain are specifically binding together to a second antigen. Methods for making bispecific antibodies are known in the art. For example, bispecific antibodies can be produced recombinantly using the co-expression of two immunoglobulin heavy chain/light chain pairs (see e.g. Milstein et al., Nature (1983), vol. 305, p. 537-539). Alternatively, bispecific antibodies can be prepared using chemical linkage (see e.g. Brennan et al., Science (1985), vol. 229, p. 81). A bispecific antibody can also for example be prepared by the SEED technology (an approach for generation of bispecific antibodies in which structurally related sequences within the conserved CH3 domains of human IgA and IgG are exchanged to form two asymmetric but complementary domains, see WO 2016/087650).

In some embodiments, said antibody is a bispecific antibody.

In some embodiments, the antibody is monovalent. In some embodiments, the antibody is bivalent. In some embodiments, the antibody is multivalent. A “monovalent” antibody/antibody component has one antigen-binding site. A “bivalent” antibody/antibody component has two antigen-binding sites. These two antigen-binding sites may bind the same or different antigens. A “multivalent” antibody/antibody component has more than two antigen-binding sites. These more than two antigen-binding sites may bind the same or different antigens.

In some embodiments, said antibody is an antibody selected from the group consisting of a chimeric antibody, a humanized antibody and a human antibody.

As used in this disclosure, a “chimeric” antibody is an antibody in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. 4,816,567; Morrison et al., Proc. Natl. Acad. Sci USA (1984), vol. 81, p. 6851-6855). As used herein, “humanized antibody” is used a subset of “chimeric antibodies.”

A “humanized antibody”, as used herein, is a “humanized” form of non-human (e.g., murine) antibody. A “humanized antibody”, is a chimeric antibody that contains minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (FR) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see e.g. Presta, Curr. Op. Struct. Biol. (1992), vol. 2, p. 593-596.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Also available for the preparation of human monoclonal antibodies are methods described in Cole et al., in: Monoclonal Antibodies and Cancer Therapy (1985), editors Reisfeld and Sell, publisher Alan R. Liss Inc. (New York), p. 77-96; van Dijk and van de Winkel, Curr. Opin. Pharmacol. (2001), vol. 5, p. 368-374. Human antibodies can be prepared by administering the antigen to a transgenic animal that has been modified to produce such antibodies in response to antigenic challenge, but whose endogenous loci have been disabled, e.g., immunized xenomice (see e.g. U.S. Pat. 6,075,181 and U.S. Pat. 6,150,584 regarding XENOMOUSE™ technology). See also, for example, Li et al., Proc. Natl. Acad. Sci. USA (2006), vol. 103, p. 3557-3562 regarding human antibodies generated via a human B-cell hybridoma technology.

The antibody component according to the present disclosure can be of any class (e.g. IgA, IgD, IgE, IgG, and IgM, preferably IgG), or subclass (e.g., IgG1, IgG2, IgG3, IgG4, IgA1 and IgA2, preferably IgG1). The different classes of immunoglobulins have different and well-known subunit structures and three-dimensional configurations (Immunobiology, 5th ed. (2001), editors Janeway et al., Garland Publishing (USA)).

In some embodiments, said antibody is a human antibody.

In some embodiments, said antibody is an antibody selected from the group consisting of an IgG antibody, an IgA antibody, an IgM antibody, and hybrids thereof.

An antibody consisting of a “hybrid” of two antibodies of different class/subclasses refers to an antibody that contains sequences from these two antibodies of different class/subclass. For example, a bispecific antibody prepared by the SEED technology (WO 2016/087650) typically contains sequences from both IgG and IgA and thus would be considered a “hybrid” of an IgG antibody and an IgA antibody.

In some embodiments, said antibody is an antibody selected from the group consisting of an IgG antibody, an IgA antibody and hybrids thereof.

In some embodiments, said antibody is an IgG antibody.

In some embodiments, said IgG antibody is an IgG1 antibody, an IgG2 antibody, an IgG3 antibody or an IgG4 antibody.

In some embodiments, said IgG antibody is an IgG1 antibody.

In some embodiments, said antibody is an antibody with the SEED (strand-exchange engineered domain) format.

In some embodiments, said targeting moiety is an antigen-binding fragment of an antibody.

In some embodiments, said antigen-binding fragment is selected from the group consisting of a Fab, a Fab′, a (Fab′)2, a Fv, a scFv, a diabody and a VHH.

“Fab” fragments are obtained by papain digestion of an antibody, which produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site.

“F(ab′)2” fragments are obtained by pepsin treatment of an antibody, which yields a single large F(ab′)2 fragment which roughly corresponds to two disulfide linked Fab fragments having different antigen-binding activity and is still capable of cross-linking antigen.

“Fab′” fragments differ from Fab fragments by having a few additional residues at the carboxy terminus of the CH1 domain including one or more cysteines from the antibody hinge region. Fab′-SH is the designation for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv”, also abbreviated as “scFv”, are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding. For a review of the scFv, see Pluckthun, in: The Pharmacology of Monoclonal Antibodies, vol. 113 (1994), editors Rosenburg and Moore, Springer-Verlag (New York), p. 269-315.

The term “diabody” refers to a small antibody fragment prepared by constructing scFv fragments (see preceding paragraph) with short linkers (about 5-10) residues) between the VH and VL domains such that inter-chain but not intra-chain pairing of the V domains is achieved, thereby resulting in a bivalent fragment, i.e., a fragment having two antigen-binding sites. Bispecific diabodies are heterodimers of two “crossover” scFv fragments in which the VH and VL domains of the two antibodies are present on different polypeptide chains. Diabodies are described in greater detail in, for example, EP 0404097; WO 93/11161; Hollinger et al., Proc. Natl. Acad. Sci. USA (1993), vol. 90, p. 6444-6448.

As used herein, the terms “VHH” and “nanobody” have the same meaning. They refer to single-domain antibodies which are antibody fragments consisting of a single monomeric variable region of a heavy chain of an antibody. Like a whole antibody, a VHH is able to bind selectively to a specific antigen. With a molecular weight of only 12-15 kDa, VHHs are much smaller than common antibodies (150-160 kDa). The first single-domain antibodies were engineered from heavy-chain antibodies found in camelids. (Gibbs and Wayt, Nanobodies, Scientific American Magazine (2005)). Generally, the antibodies with a natural deficiency of the light chain and the heavy chain constant region 1 (CH1) are first obtained, the variable regions of the heavy chain of the antibody are therefore cloned to construct a single domain antibody (VHH) consisting of only one heavy chain variable region.

In some embodiments, said antigen-binding fragment is selected from the group consisting of a Fab, a Fab′, a (Fab′)2 and a Fv.

In some embodiments, said antigen-binding fragment is selected from the group consisting of a scFv, a diabody and a VHH.

In some embodiments, said antigen-binding fragment is selected from the group consisting of a Fab, a Fab′, a (Fab′)2 and a Fv.

In some embodiments, said antigen-binding fragment is a Fab.

In some embodiments, said antigen-binding fragment is selected from the group consisting of a scFv, a diabody and a VHH.

In some embodiments, said antigen-binding fragment is an antigen-binding fragment of a monoclonal antibody or a polyclonal antibody.

In some embodiments, said antigen-binding fragment is an antigen-binding fragment of a monoclonal antibody.

In some embodiments, said antigen-binding fragment is an antigen-binding fragment of a monospecific antibody or a bispecific antibody.

In some embodiments, said antigen-binding fragment is an antigen-binding fragment of a bispecific antibody, and wherein said antigen-binding fragment is capable of binding both antigens for which said bispecific antibody is specific.

In some embodiments, said antigen-binding fragment is an antigen-binding fragment of an antibody selected from the group consisting of a chimeric antibody, a humanized antibody and a human antibody.

In some embodiments, said antigen-binding fragment is an antigen-binding fragment of a human antibody.

In some embodiments, said antigen-binding fragment is an antigen-binding fragment of an antibody selected from the group consisting of an IgG antibody, an IgA antibody, an IgM antibody, and hybrids thereof.

In some embodiments, said antigen-binding fragment is an antigen-binding fragment of an antibody selected from the group consisting of an IgG antibody, an IgA antibody and hybrids thereof.

In some embodiments, said antigen-binding fragment is an antigen-binding fragment of an IgG antibody.

In some embodiments, said IgG antibody is selected from the group consisting of an IgG1 antibody, an IgG2 antibody, an IgG3 antibody, IgG4 antibody and hybrids thereof.

In some embodiments, said IgG antibody is an IgG1 antibody.

In some embodiments, said antigen-binding fragment is an antigen-binding fragment of an antibody with the SEED (strand-exchange engineered domain) format.

In some embodiments, said targeting moiety is capable of specifically binding to an antigen that is present on the surface of a target cell.

In some embodiments, said antibody or antigen-binding fragment is capable of specifically binding to an antigen that is present on the surface of a target cell.

In some embodiments, said antibody is an antibody against an antigen that is present on the surface of a target cell.

In some embodiments, said antigen-binding fragment is an antigen-binding fragment of an antibody against an antigen that is present on the surface of a target cell.

An antibody/antigen-binding fragment “against” a certain antigen is an antibody/antigen-binding fragment with an antigen-binding site that binds to said antigen. If an antibody/antigen-binding fragment binds to an antigen can e.g. be determined by testing in an immunofluorescence experiment with cultured cells whether the antibody binds to cells that express the antigen at their cell surface.

In some embodiments, said antigen that is present on the surface of said target cell is more abundant on the surface of said target cell than on the surface of other cell types.

The abundance of a surface antigen on a cell type can be determined by standard methods known to a skilled person, e.g. flow cytometry (e.g. by exposing cell of said cell type to the antibody of interest, subsequently staining with a fluorescently labelled secondary antibody directed against the antibody of interest, and detection of fluorescent label by flow cytometry).

In some embodiments, said antigen that is present on the surface of said target cell is present on the surface of said target cell, but substantially not on the surface of other cell types.

As used herein, an antigen that is “present on the surface of said target cell, but substantially not on the surface of other cell types” is sufficiently abundant at the surface of the target cell to allow for recruitment of a compound with a targeting moiety (an antibody or antigen-binding fragment thereof) against said antigen under physiological conditions. In contrast, abundance of said antigen at the surface of other cell types is so low that recruitment of said compound under physiological conditions is barely above background binding.

In some embodiments, said antigen that is present on the surface of said target cell is present on the surface of said target cell, but not on the surface of other cell types.

As used herein, an antigen that is “present on the surface of said target cell, but not on the surface of other cell types” is sufficiently abundant at the surface of the target cell to allow for recruitment of a compound with a targeting moiety (an antibody or antigen-binding fragment thereof) against said antigen under physiological conditions. In contrast, abundance of said antigen at the surface of other cell types is so low that recruitment of said compound under physiological conditions is not above background binding.

In some embodiments, said binding of said targeting moiety to said antigen that is present on the surface of said target cell allows to recruit the compound specifically to said target cell.

In some embodiments, said binding of said antibody to said antigen that is present on the surface of said target cell allows to recruit the compound specifically to said target cell.

In some embodiments, said binding of said antigen-binding fragment to said antigen that is present on the surface of said target cell allows to recruit the compound specifically to said target cell.

The term “allows to recruit the antibody-drug conjugate specifically to said target cell” means that the compound is recruited to said target cell under physiological conditions with an efficiency that is at least 10 times higher, preferably at least 100 times higher, than the recruitment to other cell types (i.e. to other cell types to which said compound may be exposed in the body during administration of said compound).

In some embodiments, said antigen that is present on the surface of said target cell is a tumor-associated antigen or an immune cell antigen.

In some embodiments, said antigen that is present on the surface of said target cell is a tumor-associated antigen.

In some embodiments, said targeting moiety is capable of specifically binding to a tumor-associated antigen or to an immune cell antigen.

In some embodiments, said targeting moiety is capable of specifically binding to a tumor-associated antigen.

In some embodiments, said antibody or antigen-binding fragment is capable of specifically binding to a tumor-associated antigen or to an immune cell antigen.

In some embodiments, said antibody or antigen-binding fragment is capable of specifically binding to a tumor-associated antigen.

The term “tumor”, as used herein, refers to an abnormal cell mass formed by neoplastic cell growth. A tumor can be benign or malignant. Preferably, in the present disclosure the term “tumor” refers to a malignant tumor. The tumor can be, but is not limited to, a tumor present in myeloma, hematological cancers such as leukemias and lymphomas (such as B cell lymphoma, T cell lymphoma, Hodgkin’s lymphoma, non-Hodgkin’s lymphoma), hematopoietic neoplasms, thymoma, head and neck cancer, sarcoma, lung cancer, liver cancer, genitourinary cancers (such as ovarian cancer, vaginal cancer, cervical cancer, uterine cancer, bladder cancer, testicular cancer, prostate cancer or penile cancer), adenocarcinoma, breast cancer, pancreatic cancer, lung cancer, renal cancer, liver cancer, primary or metastatic melanoma, squamous cell carcinoma, basal cell carcinoma, neurological tumors including brain tumors such as astrocystomas and glioblastomas, angiosarcoma, hemangiosarcoma, head and neck cancer, thyroid carcinoma, soft tissue sarcoma, bone cancer such as bone sarcoma, vascular cancer, gastrointestinal cancer (such as gastric, stomach or colon cancer) (see Rosenberg, Ann. Rev. Med. (1996), vol. 47, p. 481-491).

As used herein, the term “cancer” refers to a malignant neoplasm. Cancer can include a hematological cancer or a solid tumor. For example, the cancer can be a leukemia (e.g., acute myeloid leukemia (AML), acute monocytic leukemia, promyelocytic leukemia, eosinophilic leukaemia, acute lymphoblastic leukemia (ALL) such as acute B lymphoblastic leukemia (BALL), chronic myelogenous leukemia (CML), chronic lymphocytic leukemia (CLL)) or lymphoma (e.g., non-Hodgkin lymphoma), myelodysplastic syndrome (MDS)″ melanoma, lung cancer (e.g., non-small cell lung cancer; NSCLC), ovarian cancer, endometrial cancer, peritoneal cancer, pancreatic cancer, breast cancer, prostate cancer, squamous cell carcinoma of the head and neck, or cervical cancer. Preferably, in the present disclosure the term “cancer” refers to a solid malignant tumor.

As used herein, a “tumor-associated antigen” is, in its broadest sense, an antigen that allows recruitment of an ADC to the site of a tumor, such that a therapeutic action or diagnostic (e.g. labelling of the tumor site) can be achieved. The tumor-associated antigen may either be an antigen that is present on the surface of the tumor cells or an antigen associated with the tumor microenvironment.

Sources for information on cell surface expression and methods to identify and verify tumor-associated antigens are known to a skilled person and described in the literature (see e.g. Bornstein, AAPS J. (2015), vol. 17(3), p. 525-534; Hong et al., BMC Syst Biol. (2018), vol. 12 (Suppl 2), p. 17; Immune Epitope Database and Analysis Resource (https://www.iedb.org); Cancer Cell Line Encyclopedia (https://portals.broadinstitute.org/ccle); OASIS Database (http://oasis-genomics.org/)).

In preferred embodiments, said tumor-associated antigen is an antigen that is present on the surface of a tumor cell. In these embodiments, the term “tumor-associated antigen” indicates an antigen that is present at the cell surface of a tumor cell and allows for distinction of the tumor cell over other cell types. A tumor-associated antigen may be part of a molecule (e.g. a protein) that is expressed by a tumor cell and accessible from the extracellular environment. A tumor-associated antigen may differ (i.e. qualitatively differ) from its counterpart in corresponding non-tumor cells (e.g., where the molecule is a protein by one or more amino acid residues). Alternatively, the tumor-associated antigen may be identical to its counterpart in corresponding non-tumor cells, but present on the surface of the tumor cells at a higher level than on the surface of corresponding non-tumor cells. For example, the tumor-associated antigen may be present only on the surface of the tumor cells, but not on the surface of non-tumor cells, or the tumor-associated antigen may be present on the surface of tumor cells at a higher level (e.g. at least 5-fold higher, preferably at least 100-fold higher) than on the surface of non-tumor cells. In an embodiment, the tumor-associated antigen is present on the surface of tumor cells at a level that is at least 1000-fold higher than on the surface of non-tumor cells.

Preferably, the tumor to which said tumor-associated antigen relates is a cancer (i.e. the tumor-associated antigen that is present on the surface of a tumor cell is present on a cancer cell).

In some embodiments, said tumor-associated antigen is an antigen that is present on the surface of a tumor cell.

In some embodiments, said tumor-associated antigen is selected from the group consisting of CD1 Ia, CD4, CD19, CD20, CD21, CD22, CD23, CD25, CD52, CD30, CD33, CD37, CD40L, CD52, CD56, CD70, CD72, CD74, CD79a, CD79b, CD138, CD163, HER2, Her3, EGFR, Mucl8, integrin, PSMA, CEA, BLys, ROR1, NaPi2b, NaPi3b, CEACAM5, Muc1, integrin avb6, Met, Trop2, BCMA, disialoganglioside GD2, B-PR1B, E16, STEAP1, 0772P, Sema 5b, ETBR, MSG783, STEAP2, Trp4, CRIPTO, FcRH1, FcRH2, NCA, IL20R-alpha, Brevican, EphB2R, ASLG659, PSCA, GEDA, BAFF-R, CXCR5, HLA-DOB, P2X5, LY64, IRTA2, TENB2, PSMA, FOLH1, STR5, SSTR1, SSTR2, SSTR3, SSTR4, TGAV, ITGB6, CA9, EGFRvlll, IL2RA, AXL, CD3Q, TNFRSF8, TNFRSF17, CTAGs, CTA; CD174/Fucosyltransferase 3 (Lewis Blood Group), CLEC14A, GRP78, HSPA5, ASG-5, ENPP3, PRR4, GCC, GUCY2C, Liv-1, SLC39A8, 5T4, NCMA1, CanAg, FOLR1, GPN B, TIM-1, HAVCR1, Mindin/RG-1, B7-H4, VTCN1, PTK7, SDC1, a claudin (preferably claudin 18.2), RON, MST1 R, EPHA2, MS4A1, TNC (Tenascin C), FAP, DKK-1, CS1/SLAMF7, ENG (Endoglin), ANXA1 (Annexin A1), VCAM-1 (CD106) and folate receptor alpha.

In some embodiments, said tumor-associated antigen is selected from the group consisting of xCT, gpNMB, carbonic anhydrase IX (CAIX), cKIT, c-MET, Tumor-associated glycoprotein 72 (TAG-72), TROP-2, TRA-1-60, TRA, TNF- alpha, TM4SF1, TIM-1, TAA, TA-MUC1 (tumor-specific epitope of mucin-1 ), Sortilin (SORT1), STn, STING, STEAP-1, SSTR2, SSEA-4, SLITRK6, SLC44A4, SLAMF7, SAIL, Receptor tyrosine kinase (RTK), ROR2, ROR1, RNF43, Prolactin Receptor (PRLR), Polymorphic epithelial mucin (PEM), Phosphatidylserine (PS), Phosphatidyl Serine, PTK7, PSMA, PD-L1, P-Cadherin, OX001L, OAcGD2, Nectin-4, NaPi2b, NOTCH3, Mesothelin (MSLN), MUC16, MTX5, MTX3, MT1-MMP, MRC2, MET, MAGE, Ly6E, Lewis Y antigen, LRRC15, LRP-1, LIV-1, LHRH, LGR5, LGALS3BP, LAMP-1, KLK2, KAAG-1, IL4R, IL7R, IL1RAP, IL-4, IL-3, IL-2, IL-13R, IGF-1R, HSP90, HLA-DR, HER-3, HER-2, Globo H, GPR20, GPC3, GPC-1, GD3, GD2, GCC, FSH, FOLR-alpha, FOLR, FLT3, FGFR3, FGFR2, FCRH5, EphA3, EphA2, EpCAM, ETBR, ENPP3, EGFRviii, EGFR, EFNA4, Dysadherin, DR5 (Death receptor 5), DPEP3, DLL3, DLK-1, DCLK1, Cripto, Cathepsin D, CanAg, CXCR5, CSP-1, CLL-1, CLDN6, CLDN18.2, CEACAM6, CEACAM5, CEA, CDH6, CD79b, CD74, CD71, CD70, CD56, CD51, CD48, CD46, CD45, CD44v6, CD40L, CD38, CD37, CD352, CD33, CD317, CD30, CD300f, CD3, CD25, CD248, CD228, CD22, CD205, CD20, CD19, CD184, CD166, CD147, CD142, CD138, CD123, CCR7, CA9, CA6, C4.4a, BCMA, B7-H4, B7, -H3, Axl, ASCT2, AMHRII, ALK, AG-7, ADAM-9, 5T4, 4-1BB.

In some embodiments, said tumor-associated antigen is selected from the group consisting of EGFR (epidermal growth factor receptor), HER2 (Human Epidermal Growth Factor Receptor 2), PD-L1 (Programmed cell death 1 ligand 1) and CD20.

Insofar as the designations of antigens indicated in the present disclosure are gene designations, these designations refer to the protein(s) encoded by said gene.

In some embodiments, said tumor-associated antigen is EGFR.

In some embodiments, said tumor-associated antigen is HER2.

In some embodiments, said tumor-associated antigen is PD-L1.

In some embodiments, said tumor-associated antigen is CD20.

In some embodiments, said antibody or antigen-binding fragment has a first and a second antigen-binding site.

In some embodiments, said first antigen-binding site is capable of specifically binding to a tumor-associated antigen and said second antigen-binding site is capable of specifically binding to a tumor-associated antigen.

In some embodiments, said first and said second antigen-binding site are capable of binding to different antigens.

In some embodiments, said compound is a bispecific molecule that binds via the protein domain of (a)/(b)/(c) to NKp30 and via its targeting domain to a tumor-associated antigen.

In some embodiments, said compound is an antibody-drug conjugate (ADC) which comprises
(i) a protein domain of (a)/(b)/(c),
(ii) an antibody or antigen-binding fragment thereof,
(iii) at least one payload wherein said at least one payload is a therapeutic agent.

As used herein, an “antibody-drug conjugate” (abbreviated “ADC”) is a molecule comprising an antibody that is conjugated (often via a linker) to a payload (“drug”). In the ADC of the present disclosure, the payload is a therapeutic agent or a detectable label. The different components of the antibody-drug conjugate are covalently linked.

By binding to its antigen, the antibody of the ADC serves as targeting moiety that can direct the ADC to its target site. For example, if the antigen of the antibody is a tumor-associated antigen, the ADC will e.g. be directed to tumor cells expressing this tumor-associated antigen at their cell surface. Upon recruitment of the ADC to the target site, the payload can mediate a therapeutic action (e.g. killing of a cancer cell, local reduction of an inflammation, local stimulation or suppression of the immune system) or, if the payload is a detectable label, the target site can be identified by detection of the detectable label.

Non-targeted drugs typically reach their site of action by whole-body distribution and passive diffusion. In contrast, ADCs are targeted compounds that are not distributed evenly across the whole body. Due to the interaction of the antibody with its target antigen, an ADC is concentrated preferentially at its site target site. Therefore, ADCs with a therapeutic agent as payload require lower dosages to be therapeutically effective, thus improving the therapeutic window.

In many cases, upon binding to its target cell an ADC will be internalized into the cell, e.g. by receptor-mediated endocytosis. If the ADC comprises a cleavable linker, the linker may be cleaved after cellular degradation (e.g. by enzymatic or chemical cleavage). Alternatively, the antibody may be degraded inside of the cell. In either case, the payload is released into the cellular interior. If the payload is a medical drug, it can then fulfill its therapeutic function inside of the cell. If the payload is a detectable label it may be detected inside of the cell.

Antibody-drug conjugates, their structure, preparation and use are described in detail e.g. in Antibody-Drug Conjugates: Fundamentals, Drug Development, and Clinical Outcomes to Target Cancer, 1st edition (2016), editors Olivier and Hurvitz, publisher John Wiley & Sons, Inc. (U.S.).

The term “payload”, as used herein, refers to a chemical moiety that is conjugated to an antibody component as part of an antibody-drug conjugate. In the antibody-drug conjugate according to the present disclosure, the payload is linked to the antibody component by covalent binding through a linker. As described above, the payload in the ADC of the present disclosure (resp. the functional moiety) is a therapeutic agent or a detectable label. Upon recruitment of the ADC to its target site by binding of the antibody component to its target antigen, the payload can fulfill its function at the target site. For example, if the antibody component is specific for the tumor-associated antigen, the payload may be a cytotoxic agent that kills tumor cells, e.g. a maytansinoid or duocarmycin. Or if the antibody component is specific for an antigen indicating inflammation, the payload may be an anti-inflammatory agent, e.g. a glucocorticoid receptor antagonist like cortisol or prednisolone. Or the payload may be a detectable agent that allows to detect the presence of the target antigen or identify the target site.

Different payloads, their preparation, conjugation and use in antibody-drug conjugates are described e.g. in Nicolaou et al., Accounts of Chemical Research (2019), vol. 52(1), p. 127-139.

A “therapeutic agent”, as used herein, is an agent that exerts an effect that is linked to a therapeutic benefit if administered to a patient (e.g. by killing a tumor cells, reducing an undesired inflammation, stimulating the activity of the immune system against an infection, or suppressing the immune response in case of an autoimmune disease). Therapeutic agents useful in accordance with the present disclosure include, but are not limited to, cytotoxic agents, anti-inflammatory agents, immunostimulatory agents and immunosuppressive agents.

In some embodiments, the therapeutic agent is a cytotoxic agent, anti-inflammatory agent, immunostimulatory agent or immunosuppressive agent.

In some preferred embodiments, the therapeutic agent is a cytotoxic agent.

As used herein, a “cytotoxic agent” is a substance that is toxic to cells (i.e. causes cell death or destruction).

In some embodiments, said compound does not comprise an antibody Fc region.

If the present disclosure states that an ADC or other compound comprises “a protein domain of (a)/(b)/(c)”, this means that the ADC or other compound comprises a protein domain of (a), a protein domain of (b) and/or a protein domain of (c). Preferably, it means that the ADC or other compound comprises a protein domain of (a) (but no protein domain or (b) or (c)) or (b) (but no protein domain or (a) or (c)) or (c) (but no protein domain or (a) or (b)).

As used herein, the term “antibody Fc region” refers to the portion of a native immunoglobulin formed by the Fc domains of its two heavy chains (which includes a heavy chain constant region 1 (CH1), a heavy chain constant region 2 (CH2) and a heavy chain constant region 3 (CH3) of an immunoglobulin, but does not include variable regions of the heavy and light chains and a light chain constant region 1 (CL1) of an immunoglobulin). A native Fc region is homodimeric. In some embodiments, the term includes variant Fc regions with one or more alterations relative to a native Fc region. An Fc region may be altered by amino acid substitutions, additions and/or deletions, linkage of additional moieties, and/or alteration of the native glycans. The term encompasses Fc regions wherein each of the constituent Fc domains is different. Examples of heterodimeric Fc regions include, without limitation, Fc regions made using the “knobs into holes” technology as described in, for example U.S. Pat. No. 8,216,805 or by the SEED technology as described in WO 2016/087650.

In some embodiments, wherein said compound comprises an antibody Fc region.

In some embodiments, said compound does not comprise an antibody Fc region competent in Fc receptor binding.

An antibody Fc region is “competent in FC receptor binding” if said antibody Fc region is capable of binding to at least one of the Fc receptors (FcyRI, FcγRII, and FcγRIII subclasses, including allelic variants and alternatively spliced forms of these receptors).

In some embodiments, said compound comprises an antibody Fc region competent in Fc receptor binding.

In some embodiments, said compound does not comprise an antibody Fc region that is not competent in Fc receptor binding.

In some embodiments, said compound comprises an antibody Fc region that is not competent in Fc receptor binding.

In some embodiments, said compound does not comprise an effector-competent Fc region.

An “effector-competent” Fc region is an Fc region having the functional ability to bind proteins and/or cells of the immune system and mediate biological effects normally induced following the binding of an antibody to a corresponding antigen. Such biological effects include e.g. the ability to bind a complement protein (e.g. C1q), resulting in activation of the classical complement system leading to the opsonisation and lysis of cell pathogens (complement-dependent cytotoxicity, CDCC). Other biological effects are endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation or control of immunoglobulin production.

In some embodiments, said compound comprises an effector-competent Fc region.

In some embodiments, said compound does not comprise an antibody Fc region capable of inducing ADCC (antibody-dependent cellular cytotoxicity).

In some embodiments, said compound comprises an antibody Fc region capable of inducing ADCC.

In some embodiments, said compound does not comprise an antibody Fc region that is not capable of inducing ADCC.

In some embodiments, said Fc region is an Fc variant of a wild-type human IgG1 Fc region, wherein the Fc variant of the wild-type human IgGl Fc region contains amino acid substitutions P329G, L234A and L235A (residues numbered according to the EU index of Kabat).

These mutations and their effects are described for example in EP2691417B1 or WO2012130831A1 (see also the Examples section of the present disclosure).

In some embodiments, said compound comprises an antibody Fc region that is not capable of inducing ADCC.

In some embodiments, said targeting moiety does not comprise an antibody Fc region.

In some embodiments, said targeting moiety comprises an antibody Fc region.

In some embodiments, said targeting moiety does not comprise an antibody Fc region competent in Fc receptor binding.

In some embodiments, said targeting moiety comprises an antibody Fc region competent in Fc receptor binding.

In some embodiments, said targeting moiety does not comprise an antibody Fc region that is not competent in Fc receptor binding.

In some embodiments, said targeting moiety comprises an antibody Fc region that is not competent in Fc receptor binding.

In some embodiments, said targeting moiety does not comprise an effector-competent Fc region.

In some embodiments, said targeting moiety comprises an effector-competent Fc region.

In some embodiments, said targeting moiety does not comprise an antibody Fc region capable of inducing ADCC (antibody-dependent cellular cytotoxicity).

In some embodiments, said targeting moiety comprises an antibody Fc region capable of inducing ADCC.

In some embodiments, said targeting moiety does not comprise an antibody Fc region that is not capable of inducing ADCC.

In some embodiments, said Fc region is an Fc variant of a wild-type human IgG1 Fc region, wherein the Fc variant of the wild-type human IgG1 Fc region contains amino acid substitutions P329G, L234A and L235A (residues numbered according to the EU index of Kabat).

In some embodiments, said targeting moiety comprises an antibody Fc region that is not capable of inducing ADCC.

In some embodiments, said antibody Fc region is a human Fc region.

In some embodiments, said antibody Fc region is an IgG Fc region.

In some embodiments, said antibody Fc region is an IgG1 Fc region.

In some embodiments, said compound is capable of inducing both FcγRIIIa signalling and positive (i.e. NK cell activating) NKp30 signalling.

In some embodiments, said compound is capable of specifically binding to NKp30 on NK cells.

In some embodiments, said compound is capable of activating NK cells by binding to NKp30 on said NK cells.

In some embodiments, binding of said compound to NKp30 on NK cells activates said NK cells.

In some embodiments, said compound is an agonist of NKp30.

In some embodiments, binding of said compound to NKp30 on NK cells allows to recruit said NK cells to said compound or said compound to said NK cells.

In some embodiments, said compound binds to NKp30 with a higher affinity (i.e. lower KD) than a comparative molecule.

In some embodiments, said compound binds to NKp30 with higher Kon rate than a comparative molecule.

In some embodiments, said compound binds to NKp30 with lower Koff rate than a comparative molecule.

Affinity and Kon/Koff rate can be measured as described in Example 1 below (see section “Biolayer interferometry”).

In some embodiments, said affinity/said Kon rate/said Koff rate is measured by kinetic measurements by biolayer interferometry at 25° C. and 1000 rpm, wherein said compound resp. comparative molecule (5 µg/mL in PBS) is loaded on a anti-human Fc biosensor for 5 min followed by 60 s rinsing the sensor with kinetics buffer (KB; PBS + 0.1% Tween-20 + 1% BSA), wherein association to human NKp30 in varying concentrations (15.6-1000 nM) is measured for 60 s followed by dissociation measurement for 180 s in KB and data is fitted using a 1:1 binding model.

In some embodiments, binding of said compound to NKp30 on NK cells activates said NK cells with greater efficiency than binding of a comparative molecule.

The efficiency of activation of NK cells can be measured as described in Example 1 below (see section “NK cell activation assay”).

In some embodiments, the efficiency of activation of NK cells is measured in an assay in which 20,000 A431 cells/well are seeded in 96-well V-bottom microtiter plates and incubated for 3 h prior addition of 100,000 NK cells/well (E:T ratio of 5:1), that had previously been treated with 100 U/ml recombinant human interleukin-2 overnight, the compound to be tested is then added at a final concentration of 85 nM, incubated for 24 h at 37° C., washed two times with PBS + 1% BSA, incubated with dead cell stain, anti-human CD56 and anti-human CD69 for 1 h on ice, washed, followed by measurement of activated NK cells based on a gating strategy involving Side scatter (SSC) vs. forward scatter (FCS) for identification of single NK cells, followed by dead cell staining and CD56 staining versus an unrelated channel for the identification of living CD56⁺ NK cells followed by gating on activated CD56⁺ CD69⁺ NK cells, wherein a compound to be tested is considered to activate NK cells if the number of activated NK cells is increased in the presence of said compound to be tested.

In some embodiments, in a ⁵¹Cr release assay said compound has improved cytotoxic activity compared to a comparative molecule.

The cytotoxic activity of a molecule can be measured as described in Example 1 below (see section “Tumor cell killing assays”).

In some embodiments, said ⁵¹Cr release assay is carried out as a 4 h ⁵¹Cr release assay, wherein human PBMCs are used as effector cells at effector-to-target cell (E:T) ratios of 80:1, and wherein a higher percent lysis indicates improved cytotoxic activity.

Percent lysis can be calculated as follows from counts per minute (cpm): % lysis = (experimental cpm-basal cpm)/(maximal cpm-basal cpm)x100 and normalized (0% = % lysis of oa_hu225-SEED-PGLALA control molecule, 100% = % lysis of Cetuximab at saturating concentration) to allow for direct comparison of affinity-matured ΔB7-H6-derived molecules between individual donors.

In some embodiments, said ⁵¹Cr release assay is carried out as a 4 h ⁵¹Cr release assay, wherein human purified NK cells are used as effector cells at effector-to-target cell (E:T) ratios of 10:1, and wherein a higher percent lysis indicates improved cytotoxic activity.

In some embodiments, said ⁵¹Cr release assay is carried out as described in Repp et al., 2011.

As used herein, the reference “Repp et al., 2011” refers to the publication R. Repp et al., “Combined Fc-protein- and Fc-glyco-engineering of scFv-Fc fusion proteins synergistically enhances CD16a binding but does not further enhance NK-cell mediated ADCC,” Journal of Immunological Methods (2011), vol. 373, p. 67-78.

In some embodiments, binding of said compound to NKp30 on NK cells results in a stronger release of interferon-y (IFN-γ) than binding of a comparative molecule.

The efficiency of activation of NK cells can be measured as described in Example 1 below (see section “Cytokine release assay”).

In some embodiments, said release of IFN-γ is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human IFN-γ is analyzed in the culture supernatant after 24 h by ELISA.

In some embodiments, binding of said compound to NKp30 on NK cells results in a stronger release of tumor necrosis factor-α (TNF-α) than binding of a comparative molecule.

In some embodiments, said release of TNF-α is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human TNF-α is analyzed in the culture supernatant after 24 h by ELISA.

In some embodiments, said comparative molecule is identical to said compound with the exception that in said comparative molecule
X₁ is S;
X₂ is G;
X₃ is F;
X₄ is G;
X₅ is V;
X₆ is T;
X₇ is L;
X₈ is K.

In some embodiments, said comparative molecule is identical to said compound with the exception that said comparative molecule does not comprise said protein domain of (a), (b) or (c), but instead comprises a protein domain with the amino acid sequence of SEQ ID NO: 1.

In some embodiments, said comparative molecule is identical to said compound with the exception that said comparative molecule does not comprise said protein domain of (a), (b) or (c), but instead comprises a protein domain with the amino acid sequence of SEQ ID NO: 2.

In another aspect, the present disclosure relates to a pharmaceutical composition comprising the compound according to any one of the aspects or embodiments described above.

Methods for preparing pharmaceutical compositions are known to a skilled person in the art (Remington: The Science and Practice of Pharmacy, 22nd ed. (2012), Pharmaceutical Press).

In some embodiments, said pharmaceutical composition comprises a pharmaceutically acceptable carrier, diluent and/or excipient.

The term “pharmaceutically acceptable” designates that said carrier, diluent or excipient is a non-toxic, inert material that is compatible with the other ingredients of the pharmaceutical composition and not harmful to the patient that the pharmaceutical composition is administered to, such that it can be used in a pharmaceutical product. Substances suitable as carriers, diluents or excipients in pharmaceutical compositions are known to a skilled person in the art (Remington: The Science and Practice of Pharmacy, 22nd ed. (2012), Pharmaceutical Press). The pharmaceutical composition may further include e.g. additional adjuvants, antioxidants, buffering agents, bulking agents, colorants, emulsifiers, fillers, flavoring agents, preservatives, stabilizers, suspending agents and/or other customary pharmaceutical auxiliaries.

In some embodiments, said pharmaceutical composition further includes at least one additional adjuvant, antioxidant, buffering agent, bulking agent, colorant, emulsifier, filler, flavoring agent, preservative, stabilizer, suspending agent and/or other customary pharmaceutical auxiliary.

In another aspect, the present disclosure relates to a compound according to any of the aspects or embodiments described above or a pharmaceutical composition according to any of the aspects or embodiments described above for use as a medicament.

In another aspect, the present disclosure relates to a compound according to any of the aspects or embodiments described above or a pharmaceutical composition according to any of the aspects or embodiments described above for use in the treatment of cancer.

In another aspect, the present disclosure relates to a compound according to any of the aspects or embodiments described above or a pharmaceutical composition according to any of the aspects or embodiments described above for use in the treatment of a malignant tumor.

In another aspect, the present disclosure relates to a compound according to any of the aspects or embodiments described above or a pharmaceutical composition according to any of the aspects or embodiments described above for use in the treatment of an inflammatory disease.

In some embodiments, said compound/said pharmaceutical composition is for use in the treatment of a human.

The production of medicaments containing the compound of the present disclosure according or a pharmaceutical composition according to the present disclosure can be performed according to well-known pharmaceutical methods. Further details on techniques for formulation and administration may be found e.g. in Remington: The Science and Practice of Pharmacy, 22nd ed. (2012), Pharmaceutical Press.

As used herein, “treatment” of a disease and “treating” a disease refers to the process of providing a subject with a pharmaceutical treatment, e.g., the administration of a drug, such that said disease is alleviated, reduced, minimized, halted or even healed, and/or such that the chances of a relapse into the disease are reduced or a relapse into the disease is even prevented.

The use of compounds in the treatment of diseases is known to a skilled person in the art (see e.g. Coats et al., Clinical Cancer Research (2019), vol. 25(18), p. 5441-5448; Rudra, Bioconjugate Chemistry (2020), vol. 31(3), p. 462-473). Thus, the skilled person is aware that the components of the compound, in particular the targeting moiety, must be selected appropriately in order to allow for successful treatment. For example, for treatment of a specific cancer, the targeting moiety of the compound must be selected such that binding of the targeting moiety to its target site directs the compound to said cancer (e.g. by using an antibody component against a tumor-associated antigen that is specifically found on the surface of the cancer cells). Cytotoxic effects will then be achieved by the affinity-matured variant B7-H6 sequence included in the compound. In addition, a payload may be included in the compound such that an additional desired treatment effect is achieved. For example, for the treatment of a cancer, a cytotoxic drug may be included in addition.

In another aspect, the present disclosure relates to a method for treating a disease in a patient in need thereof, comprising the step of administering to said patient a therapeutically effective amount of the compound according to any of the aspects or embodiments described above or the pharmaceutical composition according to any of the aspects or embodiments described above.

By “therapeutically effective amount” is meant the amount of an agent required to ameliorate the symptoms of a disease. The effective amount of active agent(s) (e.g., a compound according to the present disclosure) used for therapeutic treatment of a disease according to the present disclosure varies depending upon the manner of administration, the age, body weight, and general health of the subject. Ultimately, the attending physician or veterinarian will decide the appropriate amount and dosage regimen. Such amount is referred to as a “therapeutically effective” amount.

The term “patient”, as used herein, refers to a mammal (such as a human, rat, mouse, monkey, pig, goat, cow, horse, dog or cat). Preferably, the patient is a human.

In some embodiments, said disease is cancer.

In some embodiments, said disease is a malignant tumor.

In some embodiments, said disease is an inflammatory disease.

In some embodiments, said patient is a human.

In another aspect, the present disclosure relates to the use of the compound according to any of the aspects or embodiments described above or of the pharmaceutical composition according to any of the aspects or embodiments described above for the manufacture of a medicament.

In another aspect, the present disclosure relates to the use of the compound according to any of the aspects or embodiments described above or of the pharmaceutical composition according to any of the aspects or embodiments described above for the manufacture of a medicament for the treatment of cancer.

In another aspect, the present disclosure relates to the use of the compound according to any of the aspects or embodiments described above or of the pharmaceutical composition according to any of the aspects or embodiments described above for the manufacture of a medicament for the treatment of a malignant tumor.

In another aspect, the present disclosure relates to the use of the compound according to any of the aspects or embodiments described above or of the pharmaceutical composition according to any of the aspects or embodiments described above for the manufacture of a medicament for the treatment of an inflammatory disease.

In some embodiments, said medicament is prepared for administration to a human.

The following embodiments relate to any of the compounds or pharmaceutical compositions for use in medical treatment, methods for treating a disease in a patient in need thereof, uses for the manufacture of a medicament, or any of their embodiments described above.

In some embodiments, said inflammatory disease is an autoimmune disease.

In some embodiments, said inflammatory disease is selected from the group consisting of inflammatory bowel disease (IBD), systemic lupus erythematosus (SLE), multiple sclerosis, rheumatoid arthritis, Sjogren’s syndrome and Hidradenitis suppurativa (HS).

In some embodiments, said cancer, malignant tumor or inflammatory disease is a human disease.

In another aspect, the present disclosure relates to a method for preparing a compound with an increased affinity for NKp30 compared to a compound comprising a protein domain with the amino acid sequence of SEQ ID NO: 2:

DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPAS (SEQ ID NO: 2)

wherein said method comprises preparing a compound comprising a variant of the protein domain with the amino acid sequence of SEQ ID NO: 2 in which at least one of the following amino acid replacements has been carried out compared to the sequence of SEQ ID NO: 2:
replacement of X₁ by I, L, T, V, H, W, Y, E or Q;
replacement of X₃ by W or Y;
replacement of X₄ by D, A or Q;
replacement of X₅ by I or F;
replacement of X₇ by Y, F or V,
wherein X₁, X₃, X₄, X₅ and X₇ refer to the following positions within the sequence of SEQ ID NO: 2:

DLKVEMMAGGTQITPLNDNVTIFCNIFYSQPLNITX₁ MGITWFWKSLTFD
KEVKVFEFX₃X₄ DHQEAFRPGAIVSPWRLKSGDASLRLPGIQLEEAGEYR
CEVX₅ VTPX₇ KAQGTVQLEVVASPAS (SEQ ID NO: 51)

In some embodiments, said method comprises preparing a compound comprising a variant of the protein domain with the amino acid sequence of SEQ ID NO: 2 in which at least two of the following amino acid replacements have been carried out compared to the sequence of SEQ ID NO: 2:
replacement of X₁ by I, L, T, V, H, W, Y, E or Q;
replacement of X₃ by W or Y;
replacement of X₄ by D, A or Q;
replacement of X₅ by I or F;
X₇ is replaced by Y, F or V.

In some embodiments, said method comprises preparing a compound comprising a variant of the protein domain with the amino acid sequence of SEQ ID NO: 2 in which at least three of the following amino acid replacements have been carried out compared to the sequence of SEQ ID NO: 2:
replacement of X₁ by I, L, T, V, H, W, Y, E or Q;
replacement of X₃ by W or Y;
replacement of X₄ by D, A or Q;
replacement of X₅ by I or F;
replacement of X₇ by Y, F or V.

In some embodiments, the method comprises preparing a compound comprising a variant of the protein domain with the amino acid sequence of SEQ ID NO: 2 in which outside of the positions X₁, X₃, X₄, X₅ and X₇ up to 24 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 18 amino acids have been added, replaced or deleted compared to the sequence of SEQ ID NO: 2:

In some embodiments, the method comprises preparing a compound comprising a variant of the protein domain with the amino acid sequence of SEQ ID NO: 2 in which outside of the positions X₁, X₃, X₄, X₅ and X₇ up to 12 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 6 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 5 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 4 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 3 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 2 amino acids have been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ up to 1 amino acid has been added, replaced or deleted compared to the sequence of SEQ ID NO: 2.

In some embodiments, the method comprises preparing a compound comprising a variant of the protein domain with the amino acid sequence of SEQ ID NO: 2 in which outside of the positions X₁, X₃, X₄, X₅ and X₇ no amino acid has been added, replaced or deleted compared to the sequence of SEQ ID NO: 2.

In some embodiments, the method comprises preparing a compound comprising a variant of the protein domain with the amino acid sequence of SEQ ID NO: 2 in which the following two amino acids have not been replaced compared to the sequence of SEQ ID NO: 2:
X₆;
X₈,
wherein X₆ and X₈ refer to the following positions within the sequence of SEQ ID NO: 2:

DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVX₆ PLX₈ AQGTVQLEVVASPAS (SEQ ID NO: 52)

This means that in said variant of the protein domain with the amino acid sequence of SEQ ID NO: 2 X₆ is T and X₈ is K.

In some embodiments, the method comprises preparing a compound comprising a variant of the protein domain with the amino acid sequence of SEQ ID NO: 2 in which the following three amino acids have not been replaced compared to the sequence of SEQ ID NO: 2:
X₂;
X₆;
X₈,
wherein X₂, X₆ and X₈ refer to the following positions within the sequence of SEQ ID NO: 2:

DLKVEMMAGGTQITPLNDNVTIFCNIFYSQPLNITSMX₂ ITWFWKSLTFD
KEVKVFEFFGDHQEAFRPGAIVSPWRLKSGDASLRLPGIQLEEAGEYRCE
VVVX₆ PLX₈ AQGTVQLEVVASPAS (SEQ ID NO: 53)

This means that in said variant of the protein domain with the amino acid sequence of SEQ ID NO: 2 X₂ is G, X₆ is T and X₈ is K.

In some embodiments, an amino acid has been added if said amino acid is present in the amino acid sequence of said variant of the protein domain with the amino acid sequence of SEQ ID NO: 2, but absent at the corresponding position in SEQ ID NO: 2 (wherein positions N-terminally of the first amino acid of SEQ ID NO: 2 or C-terminally of the last amino acid of SEQ ID NO: 2 are not considered).

In some embodiments, an amino acid has been replaced by another amino acid if said amino acid is present in SEQ ID NO: 2, but absent at the corresponding position in the amino acid sequence of said variant of the protein domain with the amino acid sequence of SEQ ID NO: 2, and instead another amino acid is at the respective position present in the amino acid sequence of said variant.

In some embodiments, an amino acid has been deleted if said amino acid is present in SEQ ID NO: 2, but absent at the corresponding position of the amino acid sequence of said variant of the protein domain with the amino acid sequence of SEQ ID NO: 2.

In some embodiments, outside of said amino acid sequence of SEQ ID NO: 2 said compound comprising a protein domain with the amino acid sequence of SEQ ID NO: 2 is identical to said compound comprising a variant of the protein domain with the amino acid sequence of SEQ ID NO: 2.

In some embodiments, said compound comprising a protein domain with the amino acid sequence of SEQ ID NO: 2 is a protein consisting of said protein domain with the amino acid sequence of SEQ ID NO: 2.

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of SEQ ID NO: 4 to 15 or 17 to 50:

| | | | | | | | | |
|---|---|---|---|---|---|---|---|---|
| | X₁ | X₂ | X₃ | X₄ | X₅ | X₆ | X₇ | X₈ |
| SEQ ID NO: 4 | T | G | W | G | V | T | Y | K |
| SEQ ID NO: 5 | W | G | W | G | V | T | Y | K |
| SEQ ID NO: 6 | H | G | F | G | F | T | Y | K |
| SEQ ID NO: 7 | I | G | W | G | V | T | L | K |
| SEQ ID NO: 8 | V | G | W | G | V | T | Y | K |
| SEQ ID NO: 9 | H | G | W | G | V | T | Y | K |
| SEQ ID NO: 10 | H | G | Y | G | V | T | Y | K |
| SEQ ID NO: 11 | H | G | W | G | I | T | L | K |
| SEQ ID NO: 12 | H | G | F | G | V | T | Y | K |
| SEQ ID NO: 13 | S | G | Y | G | F | T | Y | K |
| SEQ ID NO: 14 | L | G | W | G | I | T | F | K |
| SEQ ID NO: 15 | E | G | F | G | V | T | Y | K |
| SEQ ID NO: 17 | T | G | Y | G | V | T | Y | K |
| SEQ ID NO: 18 | I | G | W | G | V | T | Y | K |
| SEQ ID NO: 19 | L | G | Y | G | V | T | Y | K |
| SEQ ID NO: 20 | S | G | Y | G | F | T | L | K |
| SEQ ID NO: 21 | Y | G | W | G | V | T | Y | K |
| SEQ ID NO: 22 | Q | G | W | G | V | T | Y | K |
| SEQ ID NO: 23 | H | G | W | G | F | T | L | K |
| SEQ ID NO: 24 | L | G | W | G | V | T | Y | K |
| SEQ ID NO: 25 | E | G | W | G | V | T | Y | K |
| SEQ ID NO: 26 | E | G | Y | G | V | T | Y | K |
| SEQ ID NO: 27 | I | G | Y | G | V | T | Y | K |
| SEQ ID NO: 28 | W | G | Y | G | V | T | Y | K |
| SEQ ID NO: 29 | T | G | W | D | V | T | Y | K |
| SEQ ID NO: 30 | T | G | W | G | I | T | Y | K |
| SEQ ID NO: 31 | H | G | W | G | V | T | L | K |
| SEQ ID NO: 32 | V | G | W | G | I | T | Y | K |
| SEQ ID NO: 33 | E | G | W | G | I | T | L | K |
| SEQ ID NO: 34 | I | G | Y | G | V | T | L | K |
| SEQ ID NO: 35 | E | G | W | G | V | T | V | K |
| SEQ ID NO: 36 | S | G | Y | G | V | T | Y | K |
| SEQ ID NO: 37 | S | G | W | D | V | T | Y | K |
| SEQ ID NO: 38 | T | G | W | G | V | T | L | K |
| SEQ ID NO: 39 | H | G | Y | A | V | T | Y | K |
| SEQ ID NO: 40 | S | G | W | G | F | T | Y | K |
| SEQ ID NO: 41 | E | G | F | G | I | T | L | K |
| SEQ ID NO: 42 | S | G | Y | G | I | T | Y | K |
| SEQ ID NO: 43 | T | G | W | G | V | T | V | K |
| SEQ ID NO: 44 | S | G | Y | G | V | T | L | K |
| SEQ ID NO: 45 | L | G | W | G | V | T | L | K |
| SEQ ID NO: 46 | T | G | W | Q | V | T | L | K |
| SEQ ID NO: 47 | H | G | Y | G | V | T | L | K |
| SEQ ID NO: 48 | S | G | W | G | V | T | Y | K |
| SEQ ID NO: 49 | T | G | F | G | V | T | Y | K |
| SEQ ID NO: 50 | E | G | Y | G | V | T | L | K |

wherein X₁ to X₈ refers to the following positions within the sequence of SEQ ID NO: 2:

DLKVEMMAGGTQITPLNDNVTIFCNIFYSQPLNITX₁ MX₂ ITWFWKSLTF
DKEVKVFE FX₃X₄ DHQEAFRPGAIVSPWRLKSGDASLRLPGIQLEEAGE
YRCEVX₅ VX₆ PX₇X₈ AQGTVQLEVVASPAS (SEQ ID NO: 3
)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 6, 8, 9, 10, 11, 12, 13, 14, 15, 17, 18, 19, 20, 21, 22, 24, 25, 26, 27, 28, 29, 30, 32, 33, 34, 36, 38, 42, 45 and 48. (As can be seen e.g. from Table 4 in Example 4, compounds with such a protein domain show a KD improvement factor ≥ 2 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 8, 9, 11, 12, 14, 17, 18, 19, 20, 21, 24, 27, 28, 32 and 34. (As can be seen e.g. from Table 4 in Example 4, compounds with such a protein domain show a KD improvement factor ≥ 5 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 8, 9, 14, 17, 18, 19, 21, 24, 27 and 32. (As can be seen e.g. from Table 4 in Example 4, compounds with such a protein domain show a KD improvement factor ≥ 10 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 8, 18, 21, 24, 27 and 32. (As can be seen e.g. from Table 4 in Example 4, compounds with such a protein domain show a KD improvement factor ≥ 15 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 21, 24 and 27. (As can be seen e.g. from Table 4 in Example 4, compounds with such a protein domain show a KD improvement factor ≥ 20 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 5, 6, 7, 8, 9, 10, 11, 12, 13, 14, 15, 17, 18, 19, 20, 21, 22, 23, 24, 25, 26, 27, 28, 29, 30, 32, 33, 34, 35, 36, 38, 39, 42, 45 and 48. (As can be seen e.g. from Table 3 in Example 4, compounds with such a protein domain show a KD improvement factor ≥ 2 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 5, 7, 8, 9, 11, 12, 14, 17, 18, 19, 20, 21, 23, 24, 27, 28, 32 and 34. (As can be seen e.g. from Table 3 in Example 4, compounds with such a protein domain show a KD improvement factor ≥ 5 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 5, 7, 8, 9, 14, 17, 18, 19, 21, 24, 27 and 32. (As can be seen e.g. from Table 3 in Example 4, compounds with such a protein domain show a KD improvement factor ≥ 10 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 5, 7, 8, 18, 21, 24, 27 and 32. (As can be seen e.g. from Table 3 in Example 4, compounds with such a protein domain show a KD improvement factor ≥ 15 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 5, 18, 21, 24 and 27. (As can be seen e.g. from Table 3 in Example 4, compounds with such a protein domain show a KD improvement factor ≥ 20 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 8, 9, 10, 11, 12, 13, 14, 17, 18, 19, 21, 24, 26, 27, 28, 29, 30, 32 and 34. (As can be seen e.g. from Table 6 in Example 5, compounds with such a protein domain show an EC50 improvement factor ≥ 10 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 9, 11, 17, 18, 19, 21, 24, 27, 28, 29, 30, 32 and 34. (As can be seen e.g. from Table 6 in Example 5, compounds with such a protein domain show an EC50 improvement factor ≥ 20 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs from the group consisting of SEQ ID NO: 17, 18, 21, 27, 28, 30, 32 and 34. (As can be seen e.g. from Table 6 in Example 5, compounds with such a protein domain show an EC50 improvement factor ≥ 40 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18 and 32. (As can be seen e.g. from Table 6 in Example 5, compounds with such a protein domain show an EC50 improvement factor ≥ 100 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 6, 8, 9, 10, 11, 12, 13, 14, 15, 17, 18, 19, 21, 22, 24, 25, 26, 27, 28, 29, 30, 31, 32, 33, 34, 36, 38, 42, 45, 46, 48, 49 and 50. (As can be seen e.g. from Table 6 in Example 5, compounds with such a protein domain show a max. killing improvement factor ≥ 1.3 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 6, 9, 10, 11, 12, 13, 15, 19, 21, 30, 31, 36, 42, 45, 48 and 49. (As can be seen e.g. from Table 6 in Example 5, compounds with such a protein domain show a max. killing improvement factor ≥ 1.5 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 13, 19, 30 and 48. (As can be seen e.g. from Table 6 in Example 5, compounds with such a protein domain show a max. killing improvement factor ≥ 1.7 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 17, 18, 19, 21, 24, 27, 28, 29, 30, 32 and 34. (As can be seen e.g. from Example 6, compounds with such a protein domain show an IFNγ release improvement factor ≥ 1 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 19, 21 and 27. (As can be seen e.g. from Example 6, compounds with such a protein domain show an IFNγ release improvement factor ≥ 3 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 17, 18, 19, 21, 24, 27, 28, 29, 30, 32 and 34. (As can be seen e.g. from Example 6, compounds with such a protein domain show a TNFα release improvement factor > 1 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 19, 21, 24, 27, 28, 30, 32 and 34. (As can be seen e.g. from Example 6, compounds with such a protein domain show a TNFα release improvement factor > 8 compared to ΔB7-H6_wt.)

In some embodiments, the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 21, 27, 30 and 34. (As can be seen e.g. from Example 6, compounds with such a protein domain show a TNFα release improvement factor > 10 compared to ΔB7-H6_wt.)

In some embodiments, said compound comprising a variant of the protein domain with the amino acid sequence of SEQ ID NO: 2 is capable of specifically binding to NKp30.

In some embodiments, said compound comprising a variant of the protein domain with the amino acid sequence of SEQ ID NO: 2 is, upon binding to NKp30 on NK cells, capable of activating NK cells.

In some embodiments, said compound comprising a variant of the protein domain with the amino acid sequence of SEQ ID NO: 2 is capable of specifically binding to NKp30 on NK cells.

In some embodiments, said compound comprising a variant of the protein domain with the amino acid sequence of SEQ ID NO: 2 is capable of activating NK cells by binding to NKp30 on said NK cells.

In some embodiments, binding of said compound comprising a variant of the protein domain with the amino acid sequence of SEQ ID NO: 2 to NKp30 on NK cells activates said NK cells.

In some embodiments, said compound comprising a variant of the protein domain with the amino acid sequence of SEQ ID NO: 2 is an agonist of NKp30.

In some embodiments, binding of said compound comprising a variant of the protein domain with the amino acid sequence of SEQ ID NO: 2 to NKp30 on NK cells allows to recruit said NK cells to said compound or said compound to said NK cells.

In some embodiments, said compound comprising a variant of the protein domain with the amino acid sequence of SEQ ID NO: 2 binds to NKp30 with a higher Kon rate than said compound comprising a protein domain with the amino acid sequence of SEQ ID NO: 2.

In some embodiments, said compound comprising a variant of the protein domain with the amino acid sequence of SEQ ID NO: 2 binds to NKp30 with a lower Koff rate than said compound comprising a protein domain with the amino acid sequence of SEQ ID NO: 2.

In some embodiments, binding of said compound comprising a variant of the protein domain with the amino acid sequence of SEQ ID NO: 2 to NKp30 on NK cells activates said NK cells with greater efficiency than binding of said compound comprising a protein domain with the amino acid sequence of SEQ ID NO: 2.

In some embodiments, the efficiency of activation of NK cells is measured in an assay in which 20,000 A431 cells/well are seeded in 96-well V-bottom microtiter plates and incubated for 3 h prior addition of 100,000 NK cells/well (E:T ratio of 5:1), that had previously been treated with 100 U/ml recombinant human interleukin-2 overnight, the compound to be tested is then added at a final concentration of 85 nM, incubated for 24 h at 37° C., washed two times with PBS + 1% BSA, incubated with dead cell stain, anti-human CD56 and anti-human CD69 for 1 h on ice, washed, followed by measurement of activated NK cells based on a gating strategy involving Side scatter (SSC) vs. forward scatter (FCS) for identification of single NK cells, followed by dead cell staining and CD56 staining versus an unrelated channel for the identification of living CD56⁺ NK cells followed by gating on activated CD56⁺ CD69⁺ NK cells, wherein a compound to be tested is considered to activate NK cells if the number of activated NK cells is increased in the presence of said compound to be tested.

In some embodiments, in a ⁵¹Cr release assay said compound comprising a variant of the protein domain with the amino acid sequence of SEQ ID NO: 2 has improved cytotoxic activity compared to said compound comprising a protein domain with the amino acid sequence of SEQ ID NO: 2.

In some embodiments, said ⁵¹Cr release assay is carried out as a 4 h ⁵¹Cr release assay, wherein human PBMCs are used as effector cells at effector-to-target cell (E:T) ratios of 80:1, and wherein a higher percent lysis indicates improved cytotoxic activity.

In some embodiments, said ⁵¹Cr release assay is carried out as a 4 h ⁵¹Cr release assay, wherein human purified NK cells are used as effector cells at effector-to-target cell (E:T) ratios of 10:1, and wherein a higher percent lysis indicates improved cytotoxic activity.

In some embodiments, said ⁵¹Cr release assay is carried out as described in Repp et al., 2011.

In some embodiments, binding of said compound comprising a variant of the protein domain with the amino acid sequence of SEQ ID NO: 2 to NKp30 on NK cells results in a stronger release of interferon-y (IFN-γ) than binding of said compound comprising a protein domain with the amino acid sequence of SEQ ID NO: 2.

In some embodiments, said release of IFN-γ is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human IFN-γ is analyzed in the culture supernatant after 24 h by ELISA.

In some embodiments, binding of said compound comprising a variant of the protein domain with the amino acid sequence of SEQ ID NO: 2 to NKp30 on NK cells results in a stronger release of tumor necrosis factor-α (TNF-α) than binding of said compound comprising a protein domain with the amino acid sequence of SEQ ID NO: 2.

In some embodiments, said release of TNF-α is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human TNF-α is analyzed in the culture supernatant after 24 h by ELISA.

The following embodiments relate to said compound, said pharmaceutical composition, said compound or pharmaceutical composition for use, said method and said use described above.

In some embodiments, said protein domain of (a) consists of an amino acid sequence selected from the group consisting of SEQ ID NO: 4, 5, 6, 7, 8, 9, 10, 11, 12, 13, 14, 15, 17, 18, 19, 20, 21, 22, 23, 24, 25, 26, 27, 28, 29, 30, 32, 33, 34, 35, 36, 38, 39, 42, 45 and 48. (As can be seen e.g. from Table 3 in Example 4, compounds with such a protein domain show a KD improvement factor ≥ 2 compared to ΔB7-H6_wt.)

In some embodiments, said protein domain of (a) consists of an amino acid sequence selected from the group consisting of SEQ ID NO: 4, 5, 7, 8, 9, 11, 12, 14, 17, 18, 19, 20, 21, 23, 24, 27, 28, 32 and 34. (As can be seen e.g. from Table 3 in Example 4, compounds with such a protein domain show a KD improvement factor ≥ 5 compared to ΔB7-H6_wt.)

In some embodiments, said protein domain of (a) consists of an amino acid sequence selected from the group consisting of SEQ ID NO: 4, 5, 7, 8, 9, 14, 17, 18, 19, 21, 24, 27 and 32. (As can be seen e.g. from Table 3 in Example 4, compounds with such a protein domain show a KD improvement factor ≥ 10 compared to ΔB7-H6_wt.)

In some embodiments, said protein domain of (a) consists of an amino acid sequence selected from the group consisting of SEQ ID NO: 5, 8, 8, 18, 21, 24, 27 and 32. (As can be seen e.g. from Table 3 in Example 4, compounds with such a protein domain show a KD improvement factor ≥ 15 compared to ΔB7-H6_wt.)

In some embodiments, said protein domain of (a) consists of an amino acid sequence selected from the group consisting of SEQ ID NO: 5, 18, 21, 24 and 27. (As can be seen e.g. from Table 3 in Example 4, compounds with such a protein domain show a KD improvement factor ≥ 20 compared to ΔB7-H6_wt.)

In some embodiments, an amino acid is added if said amino acid is present in the amino acid sequence of said protein domain of (c), but said amino acid is absent at the corresponding position of said at least one sequence listed under (a).

In some embodiments, an amino acid is replaced by another amino acid if said amino acid is present in said at least one sequence listed under (a), but absent at the corresponding position in the amino acid sequence of said protein domain of (c), and instead another amino acid is present in the amino acid sequence of said protein domain of (c) at the respective position.

In some embodiments, said replacement is a conservative amino acid replacement.

In some embodiments, an amino acid is deleted if said amino acid is present in said at least one sequence listed under (a), but absent at the corresponding position of the amino acid sequence of said protein domain of (c).

In some embodiments, none of the amino acids shown in bold in said at least one sequence listed under (a) is replaced or deleted in the amino acid sequence of said protein domain of (c).

In some embodiments, all amino acids depicted in bold in said at least one sequence listed under (a) are conserved in the amino acid sequence of said protein domain of (c).

In some embodiments, said compound binds to NKp30 with a higher affinity compared to a corresponding compound in which said protein domain is replaced by SEQ ID NO: 2, reflected by a difference in the KD by a factor of ≥ 2 (i.e. the KD is smaller by a factor ≥ 2) compared to a corresponding compound in which said protein domain is replaced by SEQ ID NO: 2.

In some embodiments, said compound binds to NKp30 with a higher affinity compared to a corresponding compound in which said protein domain is replaced by SEQ ID NO: 2, reflected by a difference in the KD by a factor of ≥ 5 (i.e. the KD is smaller by a factor ≥ 5) compared to a corresponding compound in which said protein domain is replaced by SEQ ID NO: 2.

In some embodiments, said compound binds to NKp30 with a higher affinity compared to a corresponding compound in which said protein domain is replaced by SEQ ID NO: 2, reflected by a difference in the KD by a factor of ≥ 10 (i.e. the KD is smaller by a factor ≥ 10) compared to a corresponding compound in which said protein domain is replaced by SEQ ID NO: 2.

In some embodiments, said compound binds to NKp30 with a higher affinity compared to a corresponding compound in which said protein domain is replaced by SEQ ID NO: 2, reflected by a difference in the KD by a factor of ≥ 15 (i.e. the KD is smaller by a factor ≥ 15) compared to a corresponding compound in which said protein domain is replaced by SEQ ID NO: 2.

In some embodiments, said compound binds to NKp30 with a higher affinity compared to a corresponding compound in which said protein domain is replaced by SEQ ID NO: 2, reflected by a difference in the KD by a factor of ≥ 20 (i.e. the KD is smaller by a factor ≥ 20) compared to a corresponding compound in which said protein domain is replaced by SEQ ID NO: 2.

In some embodiments, the half maximal effective concentration (EC50) of said compound for NK cell activation in the assay according to item [253] is by a factor of ≥ 10 lower compared to a corresponding compound in which said protein domain is replaced by SEQ ID NO: 2.

In some embodiments, the half maximal effective concentration (EC50) of said compound for NK cell activation in the assay according to item [253] is by a factor of ≥ 20 lower compared to a corresponding compound in which said protein domain is replaced by SEQ ID NO: 2.

In some embodiments, the half maximal effective concentration (EC50) of said compound for NK cell activation in the assay according to item [253] is by a factor of ≥ 40 lower compared to a corresponding compound in which said protein domain is replaced by SEQ ID NO: 2.

In some embodiments, the half maximal effective concentration (EC50) of said compound for NK cell activation in the assay according to item [253] is by a factor of ≥ 100 lower compared to a corresponding compound in which said protein domain is replaced by SEQ ID NO: 2.

In some embodiments, the maximal killing efficiency of said compound is by a factor of ≥ 1.3 higher compared to a corresponding compound in which said protein domain is replaced by SEQ ID NO: 2, wherein the maximal killing efficiency is measured by a 4 h ⁵¹Cr release assay, wherein human PBMCs are used as effector cells at effector-to-target cell (E:T) ratios of 80:1, and wherein killing efficiency is measured as percent lysis.

In some embodiments, the maximal killing efficiency of said compound is by a factor of ≥ 1.5 higher compared to a corresponding compound in which said protein domain is replaced by SEQ ID NO: 2, wherein the maximal killing efficiency is measured by a 4 h ⁵¹Cr release assay, wherein human PBMCs are used as effector cells at effector-to-target cell (E:T) ratios of 80:1, and wherein killing efficiency is measured as percent lysis.

In some embodiments, the maximal killing efficiency of said compound is by a factor of ≥ 1.7 higher compared to a corresponding compound in which said protein domain is replaced by SEQ ID NO: 2, wherein the maximal killing efficiency is measured by a 4 h ⁵¹Cr release assay, wherein human PBMCs are used as effector cells at effector-to-target cell (E:T) ratios of 80:1, and wherein killing efficiency is measured as percent lysis.

In some embodiments, binding of said compound to NKp30 on NK cells results in a release of interferon-y (IFN-γ) that is higher (i.e. by a factor of > 1 increased) compared to a corresponding compound in which said protein domain is replaced by SEQ ID NO: 2, as measured according to item [259].

In some embodiments, binding of said compound to NKp30 on NK cells results in a release of interferon-y (IFN-γ) that is by a factor > 3 higher compared to a corresponding compound in which said protein domain is replaced by SEQ ID NO: 2, as measured according to item [259].

In some embodiments, binding of said compound to NKp30 on NK cells results in a release of tumor necrosis factor-α (TNF-α) that is higher (i.e. by a factor of > 1 increased) compared to a corresponding compound in which said protein domain is replaced by SEQ ID NO: 2, as measured according to item [261].

In some embodiments, binding of said compound to NKp30 on NK cells results in a release of tumor necrosis factor-α (TNF-α) that is by a factor > 8 higher compared to a corresponding compound in which said protein domain is replaced by SEQ ID NO: 2, as measured according to item [261].

In some embodiments, binding of said compound to NKp30 on NK cells results in a release of tumor necrosis factor-α (TNF-α) that is by a factor > 10 higher compared to a corresponding compound in which said protein domain is replaced by SEQ ID NO: 2, as measured according to item [261].

Also disclosed with regard to the above-described subject matter is the following:
A compound comprising
   (a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO:
3),

wherein
   X₁ is I, L, T, V, H, W or Y;
   X₂ is G;
   X₃ is W or Y;
   X₄ is G;
   X₅ is V or I;
   X₆ is T;
   X₇ is Y, F or L;
   X₈ is K;
(b) a protein domain which consists of an amino acid sequence that is at least 75% identical to the amino acid sequence of the protein domain of (a); or
(c) a protein domain which is a fragment of the protein domain of (a) or (b).
The compound according to item [1], wherein in said protein domain of (a) X₁ is I, L, T or V.
The compound according to any one of items [1] to [2], wherein in said protein domain of (a) X₃ is W.
The compound according to any one of items [1] to [3], wherein in said protein domain of (a) X₅ is V.
The compound according to any one of items [1] to [4], wherein in said protein domain of (a) X₇ is Y.
A compound comprising
   (a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3)
,

wherein
   X₁ is H, E, S, T, I or W;
   X₂ is G;
   X₃ is Y, W or F;
   X₄ is G or D;
   X₅ is V, F or I;
   X₆ is T;
   X₇ is Y or L;
   X₈ is K;
(b) a protein domain which consists of an amino acid sequence that is at least 75% identical to the amino acid sequence of the protein domain of (a); or
(c) a protein domain which is a fragment of the protein domain of (a) or (b).
The compound according to item [6], wherein in said protein domain of (a) X₁ is H, E, S or T.
The compound according to any one of items [6] to [7], wherein in said protein domain of (a) X₃ is Y or W.
The compound according to any one of items [6] to [8], wherein in said protein domain of (a) X₄ is G.
The compound according to any one of items [6] to [9], wherein in said protein domain of (a) X₅ is V.
The compound according to any one of items [6] to [10], wherein in said protein domain of (a) X₇ is Y.
A compound comprising
   (a) a protein domain which consists of the amino acid sequence of SEQ ID NO: 3:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3)
,

wherein
   X₁ is E, S, H, T, L or Q;
   X₂ is G;
   X₃ is W, Y or F;
   X₄ is G, A, D or Q;
   X₅ is V, I or F;
   X₆ is T;
   X₇ is Y, L or V;
   X₈ is K;
(b) a protein domain which consists of an amino acid sequence that is at least 75% identical to the amino acid sequence of the protein domain of (a); or
(c) a protein domain which is a fragment of the protein domain of (a) or (b).
The compound according to item [12], wherein in said protein domain of (a) X₁ is E, S, H or T.
The compound according to any one of items [12] to [13], wherein in said protein domain of (a) X₃ is W.
The compound according to any one of items [12] to [14], wherein in said protein domain of (a) X₄ is G.
The compound according to any one of items [12] to [15], wherein in said protein domain of (a) X₅ is V.
The compound according to any one of items [12] to [16], wherein in said protein domain of (a) X₇ is Y or L.
The compound according to any one of items [1] to [17], wherein said protein domain of (b) is capable of specifically binding to NKp30.
The compound according to any one of items [1] to [18], wherein said protein domain of (b) is, upon binding to NKp30 on NK cells, capable of activating NK cells.
The compound according to item [19], wherein activation of NK cells is measured in an assay in which 20,000 A431 cells/well are seeded in 96-well V-bottom microtiter plates and incubated for 3 h prior addition of 100,000 NK cells/well (E:T ratio of 5:1), that had previously been treated with 100 U/ml recombinant human interleukin-2 overnight, said compound to be tested is then added at a final concentration of 85 nM, the cells are incubated for 24 h at 37° C., washed two times with PBS + 1% BSA, incubated with dead cell stain, anti-human CD56 and anti-human CD69 for 1 h on ice, washed, followed by measurement of activated NK cells based on a gating strategy involving Side scatter (SSC) vs. forward scatter (FCS) for identification of single NK cells, followed by dead cell staining and CD56 staining versus an unrelated channel for the identification of living CD56⁺ NK cells followed by gating on activated CD56⁺ CD69⁺ NK cells, wherein a compound to be tested is considered to activate NK cells if the number of activated NK cells is increased in the presence of said compound to be tested.
The compound according to any one of items [1] to [20], wherein said protein domain of (b) consists of an amino acid sequence that is at least 80% identical to the amino acid sequence of the protein domain of (a).
The compound according to any one of items [1] to [21], wherein said protein domain of (b) consists of an amino acid sequence that is at least 85% identical to the amino acid sequence of the protein domain of (a).
The compound according to any one of items [1] to [22], wherein said protein domain of (b) consists of an amino acid sequence that is at least 90% identical to the amino acid sequence of the protein domain of (a).
The compound according to any one of items [1] to [23], wherein said protein domain of (b) consists of an amino acid sequence that is at least 95% identical to the amino acid sequence of the protein domain of (a).
The compound according to any one of items [1] to [24], wherein said protein domain of (b) consists of an amino acid sequence that is at least 98% identical to the amino acid sequence of the protein domain of (a).
The compound according to any one of items [1] to [25], wherein said protein domain of (b) consists of an amino acid sequence that is at least 99% identical to the amino acid sequence of the protein domain of (a).
The compound according to any one of items [1] to [26], wherein said protein domain of (b) has at the positions corresponding to the positions of X₁, X₂, X₃, X₄, X₅, X₆, X₇ and X₈ of SEQ ID NO: 3 the same residues as defined for the protein domain of (a).
The compound according to any one of items [1] to [27], wherein in said protein domain of (b) the amino acids X₁, X₂, X₃, X₄, X₅, X₆, X₇ and X₈ are as defined in (a).
The compound according to any one of items [1] to [28], wherein in an alignment of said protein domain of (b) with SEQ ID NO: 3, the amino acids of the protein domain of (b) corresponding to X₁, X₂, X₃, X₄, X₅, X₆, X₇ and X₈ in SEQ ID NO: 3 are as defined in (a).
The compound according to any one of items [1] to [29], wherein said protein domain of (b) has
   at the amino acid position of the protein domain of (b) that corresponds to the position of X₁ in SEQ ID NO: 3 (in an alignment of the protein domain of (b) with SEQ ID NO: 3): an amino acid that is identical to the amino acid/one of the amino acids in the definition of X₁ in the protein domain of (a);
   at the amino acid position of the protein domain of (b) that corresponds to the position of X₂ in SEQ ID NO: 3 (in an alignment of the protein domain of (b) with SEQ ID NO: 3): an amino acid that is identical to the amino acid/one of the amino acids in the definition of X₂ in the protein domain of (a);
   at the amino acid position of the protein domain of (b) that corresponds to the position of X₃ in SEQ ID NO: 3 (in an alignment of the protein domain of (b) with SEQ ID NO: 3): an amino acid that is identical to the amino acid/one of the amino acids in the definition of X₃ in the protein domain of (a);
   at the amino acid position of the protein domain of (b) that corresponds to the position of X₄ in SEQ ID NO: 3 (in an alignment of the protein domain of (b) with SEQ ID NO: 3): an amino acid that is identical to the amino acid/one of the amino acids in the definition of X₄ in the protein domain of (a);
   at the amino acid position of the protein domain of (b) that corresponds to the position of X₅ in SEQ ID NO: 3 (in an alignment of the protein domain of (b) with SEQ ID NO: 3): an amino acid that is identical to the amino acid/one of the amino acids in the definition of X₅ in the protein domain of (a);
   at the amino acid position of the protein domain of (b) that corresponds to the position of X₆ in SEQ ID NO: 3 (in an alignment of the protein domain of (b) with SEQ ID NO: 3): an amino acid that is identical to the amino acid/one of the amino acids in the definition of X₆ in the protein domain of (a);
   at the amino acid position of the protein domain of (b) that corresponds to the position of X₇ in SEQ ID NO: 3 (in an alignment of the protein domain of (b) with SEQ ID NO: 3): an amino acid that is identical to the amino acid/one of the amino acids in the definition of X₇ in the protein domain of (a);
   at the amino acid position of the protein domain of (b) that corresponds to the position of X₈ in SEQ ID NO: 3 (in an alignment of the protein domain of (b) with SEQ ID NO: 3): an amino acid that is identical to the amino acid/one of the amino acids in the definition of X₈ in the protein domain of (a).
The compound according to any one of items [1] to [30], wherein the amino acids X₁, X₂, X₃, X₄, X₅, X₆, X₇ and X₈ of the protein domain of (a) are not replaced by another amino acid or deleted in said protein domain of (b) compared to said protein domain of (a).
A compound comprising
   (a) a protein domain which consists of the amino acid sequence of any one of the following sequences:

| | |
|---|---|
| SEQ ID NO | Amino acid sequence |
| SEQ ID NO: 4 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 5 | DLKVEMMAGGTQITPLNDNVTIFCNIFYSQPLNITWMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 6 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVFVTPYKAQGTVQLEVVASPAS |
| SEQ ID NO: 7 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 8 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 9 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 10 | DLKVEMMAGGTQITPLNDNVTIFCNIFYSQPLNITHMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 11 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVIVTPLKAQGTVQLEVVASPAS |
| SEQ ID NO: 12 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 13 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPYKAQGTVQLEVVASPAS |
| SEQ ID NO: 14 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVIVTPFKAQGTVQLEVVASPAS |
| SEQ ID NO: 15 | DLKVEMMAGGTQITPLNDNVTIFCNIFYSQPLNITEMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 17 | DLKVEMMAGGTQITPLNDNVTIFCNIFYSQPLNITTMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 18 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 19 | DLKVEMMAGGTQITPLNDNVTIFCNIFYSQPLNITLMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 20 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPLKAQGTVQLEVVASPAS |
| SEQ ID NO: 21 | DLKVEMMAGGTQITPLNDNVTIFCNIFYSQPLNITYMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 22 | DLKVEMMAGGTQITPLNDNVTIFCNIFYSQPLNITQMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 23 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVFVTPLKAQGTVQLEVVASPAS |
| SEQ ID NO: 24 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 25 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 26 | DLKVEMMAGGTQITPLNDNVTIFCNIFYSQPLNITEMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 27 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 28 | DLKVEMMAGGTQITPLNDNVTIFCNIFYSQPLNITWMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 29 | DLKVEMMAGGTQITPLNDNVTIFCNIFYSQPLNITTMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 30 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 31 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 32 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 33 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVIVTPLKAQGTVQLEVVASPAS |
| SEQ ID NO: 34 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 35 | DLKVEMMAGGTQITPLNDNVTIFCNIFYSQPLNITEMGITW FWKSLTFDKEVKVFEFWGDHQEAFRPGAIVSPWRLKSGDAS LRLPGIQLEEAGEYRCEVVVTPVKAQGTVQLEVVASPAS |
| SEQ ID NO: 36 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 37 | DLKVEMMAGGTQITPLNDNVTIFCNIFYSQPLNITSMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 38 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 39 | DLKVEMMAGGTQITPLNDNVTIFCNIFYSQPLNITHMGITW FWKSLTFDKEVKVFEFYADHQEAIRPGAIVSPWRLKSGDAS LRLPGIQLEEAGEYRCEVVVTPYKAQGTVQLEVVASPAS |
| SEQ ID NO: 40 | DLKVEMMAGGTQITPLNDNVTIFCNIFYSQPLNITSMGITW LWKSLTFDKEVKVFEFWGDHQEAFRPGAIVSPWRLKSGDAS LRLPGIQLEEAGEYRCEVFVTPYKAQGTVQLEVVASPAS |
| SEQ ID NO: 41 | DLKVEMMAGGTQITPLNDNVTIFCNIFYFQPLNITEMGITW FWKSLTFDKEVKVFEFFGDHQEAFRPGAIVSPWRLKSGDAS LRLPGIQLEEAGEYRCEVIVTPLKAQGTVQLEVVASPAS |
| SEQ ID NO: 42 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVIVTPYKAQGTVQLEVVTSPAS |
| SEQ ID NO: 43 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCGVVVTPVKAQGTVQLEVVASPAS |
| SEQ ID NO: 44 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVVVTPLKAQGTAQLEVVASPAS |
| SEQ ID NO: 45 | DLKVEMMAGGTQITPLNDNVTIFCNIFYSQPLNITLMGITW FWKSLTFDKEVKVFEFWGDHQEAFRPGAIVSPWGLKSGDAS LRLPGIQLEEAGEYRCEVVVTPLKAQGTVQLEVVASPAS |
| SEQ ID NO: 46 | DLKVEMMAGGTQITPLNDNVTIFCNIFYSQPLNITTMGITW FWKSLTFDKEVKVFEFWQDHQEAFRPGAIVSPWRLKSGDAS LRLPGIQLEEAGEYRCEVVVTPLKAQGTVQLEVVASPAS |
| SEQ ID NO: 47 | DLKVEMMAGGTQITPLNDNVTIFCNIFYSQPLNITHMGITW LWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 48 | DLKVEMMAGGTQITPLNDNVTIFCNIFYSQPLNITSMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 49 | DLKVEMMAGGTQITPLNDNVTIFCNIFYSQPLNITTMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 50 | DLKVEMMAGGTQITPLNDNVTIFCNIFYSQPLNITEMGITW FWKSLTFDKEVKVFEFYGDHQEAFXPGAIVSPWRLKSGDAS LXLPGIQLEEAGEYRCEVVVTPLKAQGTVQLEVVASPAS |

(b) a protein domain which consists of an amino acid sequence that is identical to at least one sequence listed under (a) with the only difference that in up to 30 occasions an amino acid is individually added, replaced by another amino acid or deleted in the amino acid sequence of the protein domain of (b) compared to said at least one sequence listed under (a).
(c) a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 30 occasions an amino acid is individually added, replaced by another amino acid or deleted in the amino acid sequence of the protein domain of (c) compared to said at least one sequence listed under (a).
The compound according to item [32], wherein said protein domain of (a) consists of the amino acid sequence of any one of the sequences listed under (a).
The compound according to any one of items [32] to [33], wherein said protein domain of (b) consists of an amino acid sequence that is identical to at least one sequence listed under (a) with the only difference that in up to 30 occasions an amino acid is individually added, replaced by another amino acid or deleted in the amino acid sequence of the protein domain of (b) compared to said at least one sequence listed under (a).
The compound according to any one of items [32] to [34], wherein said protein domain of (b) consists of an amino acid sequence that is identical to at least one sequence listed under (a) with the only difference that in up to 24 occasions an amino acid is individually added, replaced by another amino acid or deleted in the amino acid sequence of the protein domain of (b) compared to said at least one sequence listed under (a).
The compound according to any one of items [32] to [35], wherein said protein domain of (b) consists of an amino acid sequence that is identical to at least one sequence listed under (a) with the only difference that in up to 18 occasions an amino acid is individually added, replaced by another amino acid or deleted in the amino acid sequence of the protein domain of (b) compared to said at least one sequence listed under (a).
The compound according to any one of items [32] to [36], wherein said protein domain of (b) consists of an amino acid sequence that is identical to at least one sequence listed under (a) with the only difference that in up to 12 occasions an amino acid is individually added, replaced by another amino acid or deleted in the amino acid sequence of the protein domain of (b) compared to said at least one sequence listed under (a).
The compound according to any one of items [32] to [37], wherein said protein domain of (b) consists of an amino acid sequence that is identical to at least one sequence listed under (a) with the only difference that in up to 6 occasions an amino acid is individually added, replaced by another amino acid or deleted in the amino acid sequence of the protein domain of (b) compared to said at least one sequence listed under (a).
The compound according to any one of items [32] to [38], wherein said protein domain of (b) consists of an amino acid sequence that is identical to at least one sequence listed under (a) with the only difference that in up to 5 occasions an amino acid is individually added, replaced by another amino acid or deleted in the amino acid sequence of the protein domain of (b) compared to said at least one sequence listed under (a).
The compound according to any one of items [32] to [39], wherein said protein domain of (b) consists of an amino acid sequence that is identical to at least one sequence listed under (a) with the only difference that in up to 4 occasions an amino acid is individually added, replaced by another amino acid or deleted in the amino acid sequence of the protein domain of (b) compared to said at least one sequence listed under (a).
The compound according to any one of items [32] to [40], wherein said protein domain of (b) consists of an amino acid sequence that is identical to at least one sequence listed under (a) with the only difference that in up to 3 occasions an amino acid is individually added, replaced by another amino acid or deleted in the amino acid sequence of the protein domain of (b) compared to said at least one sequence listed under (a).
The compound according to any one of items [32] to [41], wherein said protein domain of (b) consists of an amino acid sequence that is identical to at least one sequence listed under (a) with the only difference that in up to 2 occasions an amino acid is individually added, replaced by another amino acid or deleted in the amino acid sequence of the protein domain of (b) compared to said at least one sequence listed under (a).
The compound according to any one of items [32] to [42], wherein said protein domain of (b) consists of an amino acid sequence that is identical to at least one sequence listed under (a) with the only difference that in up to 1 occasion an amino acid is individually added, replaced by another amino acid or deleted in the amino acid sequence of the protein domain of (b) compared to said at least one sequence listed under (a).
The compound according to any one of items [32] to [43], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 24 occasions an amino acid is individually added, replaced by another amino acid or deleted in the amino acid sequence of the protein domain of (c) compared to said at least one sequence listed under (a).
The compound according to any one of items [32] to [44], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 18 occasions an amino acid is individually added, replaced by another amino acid or deleted in the amino acid sequence of the protein domain of (c) compared to said at least one sequence listed under (a).
The compound according to any one of items [32] to [45], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 12 occasions an amino acid is individually added, replaced by another amino acid or deleted in the amino acid sequence of the protein domain of (c) compared to said at least one sequence listed under (a).
The compound according to any one of items [32] to [46], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 6 occasions an amino acid is individually added, replaced by another amino acid or deleted in the amino acid sequence of the protein domain of (c) compared to said at least one sequence listed under (a).
The compound according to any one of items [32] to [47], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 5 occasions an amino acid is individually added, replaced by another amino acid or deleted in the amino acid sequence of the protein domain of (c) compared to said at least one sequence listed under (a).
The compound according to any one of items [32] to [48], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 4 occasions an amino acid is individually added, replaced by another amino acid or deleted in the amino acid sequence of the protein domain of (c) compared to said at least one sequence listed under (a).
The compound according to any one of items [32] to [49], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 3 occasions an amino acid is individually added, replaced by another amino acid or deleted in the amino acid sequence of the protein domain of (c) compared to said at least one sequence listed under (a).
The compound according to any one of items [32] to [50], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 2 occasions an amino acid is individually added, replaced by another amino acid or deleted in the amino acid sequence of the protein domain of (c) compared to said at least one sequence listed under (a).
The compound according to any one of items [32] to [51], wherein said protein domain of (c) is a protein domain which consists of an amino acid sequence that is a fragment of the amino acid sequence of any one of the sequences listed in (a), or which consists of an amino acid sequence that is a fragment of an amino acid sequence that is identical to at least one sequence listed under (a) with the only difference that in up to 1 occasion an amino acid is individually added, replaced by another amino acid or deleted in the amino acid sequence of the protein domain of (c) compared to said at least one sequence listed under (a).
The compound according to any one of items [32] to [52], wherein said protein domain of (c) is a protein domain which consists of an amino acid sequence that is a fragment of the amino acid sequence of any one of the sequences listed in (a).
The compound according to any one of items [32] to [53], wherein an amino acid is added if said amino acid is present in the amino acid sequence of said protein domain of (b), but said amino acid is absent at the corresponding position of said at least one sequence listed under (a).
The compound according to any one of items [32] to [54], wherein an amino acid is replaced by another amino acid if said amino acid is present in said at least one sequence listed under (a), but absent at the corresponding position in the amino acid sequence of said protein domain of (b), and instead another amino acid is present in the amino acid sequence of said protein domain of (b) at the respective position.
The compound according to any one of items [32] to [55], wherein said replacement is a conservative amino acid replacement.
The compound according to any one of items [32] to [56], wherein an amino acid is deleted if said amino acid is present in said at least one sequence listed under (a), but absent at the corresponding position of the amino acid sequence of said protein domain of (b).
The compound according to any one of items [32] to [57], wherein none of the amino acids shown in bold in said at least one sequence listed under (a) is replaced or deleted in the amino acid sequence of said protein domain of (b).
The compound according to any one of items [32] to [58], wherein all amino acids depicted in bold in said at least one sequence listed under (a) are conserved in the amino acid sequence of said protein domain of (b).
The compound according to any one of items [1] to [59], wherein said protein domain of (c) is capable of specifically binding to NKp30.
The compound according to any one of items [1] to [60], wherein said protein domain of (c) is, upon binding to NKp30 on NK cells, capable of activating said NK cells.
The compound according to any one of items [1] to [61], wherein said protein domain of (c) consists of at least 75 amino acids.
The compound according to any one of items [1] to [62], wherein said protein domain of (c) consists of at least 80 amino acids.
The compound according to any one of items [1] to [63], wherein said protein domain of (c) consists of at least 90 amino acids.
The compound according to any one of items [1] to [64], wherein said protein domain of (c) consists of at least 100 amino acids.
The compound according to any one of items [1] to [65], wherein said protein domain of (c) consists of at least 110 amino acids.
The compound according to any one of items [1] to [66], wherein said protein domain of (c) consists of at least 120 amino acids.
The compound according to any one of items [1] to [67], wherein said protein domain of (c) consists of an amino acid sequence corresponding to the amino acid sequence from amino acid 55 to amino acid 135 of SEQ ID NO: 1.
The compound according to any one of items [1] to [68], wherein said protein domain of (c) consists of an amino acid sequence corresponding to the amino acid sequence from amino acid 50 to amino acid 140 of SEQ ID NO: 1.
The compound according to any one of items [1] to [69], wherein said protein domain of (c) is a fragment of the protein domain of (a).
The compound according to any one of items [1] to [70], wherein said protein domain of (a), (b) or (c) does not comprise the amino acid sequence of SEQ ID NO: 2.
The compound according to any one of items [1] to [71], wherein said compound does not comprise the amino acid sequence of SEQ ID NO: 2.
The compound according to any one of items [1] to [31] or [60] to [72], wherein in said protein domain of (a) at least one of the following does not apply:
   X₁ is S;
   X₃ is F;
   X₄ is G;
   X₅ is V;
   X₇ is L.
The compound according to any one of items [1] to [31] or [60] to [73], wherein in said protein domain of (a) at least two of the following do not apply:
   X₁ is S;
   X₃ is F;
   X₄ is G;
   X₅ is V;
   X₇ is L.
The compound according to any one of items [1] to [31] or [60] to [74], wherein in said protein domain of (a) at least three of the following do not apply:
   X₁ is S;
   X₃ is F;
   X₄ is G;
   X₅ is V;
   X₇ is L.
The compound according to any one of items [1] to [75], wherein said protein domain of (a) consists of an amino acid sequence selected from the group consisting of SEQ ID NO: 4, 6, 8, 9, 10, 11, 12, 13, 14, 15, 17, 18, 19, 20, 21, 22, 24, 25, 26, 27, 28, 29, 30, 32, 33, 34, 36, 38, 42, 45 and 48. (As can be seen e.g. from Table 4 in Example 4, compounds with such a protein domain show a KD improvement factor ≥ 2 compared to ΔB7-H6_wt.)
The compound according to any one of items [1] to [76], wherein said protein domain of (a) consists of an amino acid sequence selected from the group consisting of SEQ ID NO: 4, 8, 9, 11, 12, 14, 17, 18, 19, 20, 21, 24, 27, 28, 32 and 34. (As can be seen e.g. from Table 4 in Example 4, compounds with such a protein domain show a KD improvement factor ≥ 5 compared to ΔB7-H6_wt.)
The compound according to any one of items [1] to [77], wherein said protein domain of (a) consists of an amino acid sequence selected from the group consisting of SEQ ID NO: 4, 8, 9, 14, 17, 18, 19, 21, 24, 27 and 32. (As can be seen e.g. from Table 4 in Example 4, compounds with such a protein domain show a KD improvement factor ≥ 10 compared to ΔB7-H6_wt.)
The compound according to any one of items [1] to [78], wherein said protein domain of (a) consists of an amino acid sequence selected from the group consisting of SEQ ID NO: 8, 18, 21, 24, 27 and 32. (As can be seen e.g. from Table 4 in Example 4, compounds with such a protein domain show a KD improvement factor ≥ 15 compared to ΔB7-H6_wt.)
The compound according to any one of items [1] to [79], wherein said protein domain of (a) consists of an amino acid sequence selected from the group consisting of SEQ ID NO: 18, 21, 24 and 27. (As can be seen e.g. from Table 4 in Example 4, compounds with such a protein domain show a KD improvement factor ≥ 20 compared to ΔB7-H6_wt.)
The compound according to any one of items [1] to [80], wherein said protein domain of (a) consists of an amino acid sequence selected from the group consisting of SEQ ID NO: 4, 8, 9, 10, 11, 12, 13, 14, 17, 18, 19, 21, 24, 26, 27, 28, 29, 30, 32 and 34. (As can be seen e.g. from Table 6 in Example 5, compounds with such a protein domain show an EC50 improvement factor ≥ 10 compared to ΔB7-H6_wt.)
The compound according to any one of items [1] to [81], wherein said protein domain of (a) consists of an amino acid sequence selected from the group consisting of SEQ ID NO: 9, 11, 17, 18, 19, 21, 24, 27, 28, 29, 30, 32 and 34. (As can be seen e.g. from Table 6 in Example 5, compounds with such a protein domain show an EC50 improvement factor ≥ 20 compared to ΔB7-H6_wt.)
The compound according to any one of items [1] to [82], wherein said protein domain of (a) consists of an amino acid sequence selected from the group consisting of SEQ ID NO: 17, 18, 21, 27, 28, 30, 32 and 34. (As can be seen e.g. from Table 6 in Example 5, compounds with such a protein domain show an EC50 improvement factor ≥ 40 compared to ΔB7-H6_wt.)
The compound according to any one of items [1] to [83], wherein said protein domain of (a) consists of an amino acid sequence selected from the group consisting of SEQ ID NO: 18 and 32. (As can be seen e.g. from Table 6 in Example 5, compounds with such a protein domain show an EC50 improvement factor ≥ 100 compared to ΔB7-H6_wt.)
The compound according to any one of items [1] to [84], wherein said protein domain of (a) consists of an amino acid sequence selected from the group consisting of SEQ ID NO: 4, 6, 8, 9, 10, 11, 12, 13, 14, 15, 17, 18, 19, 21, 22, 24, 25, 26, 27, 28, 29, 30, 31, 33, 34, 36, 38, 42, 45, 46, 48, 49 and 50. (As can be seen e.g. from Table 6 in Example 5, compounds with such a protein domain show a max. killing improvement factor ≥ 1.3 compared to ΔB7-H6_wt.)
The compound according to any one of items [1] to [85], wherein said protein domain of (a) consists of an amino acid sequence selected from the group consisting of SEQ ID NO: 4, 6, 9, 10, 11, 12, 13, 15, 19, 21, 30, 31, 36, 42, 45, 48 and 49. (As can be seen e.g. from Table 6 in Example 5, compounds with such a protein domain show a max. killing improvement factor ≥ 1.5 compared to ΔB7-H6_wt.)
The compound according to any one of items [1] to [86], wherein said protein domain of (a) consists of an amino acid sequence selected from the group consisting of SEQ ID NO: 13, 19, 30 and 48. (As can be seen e.g. from Table 6 in Example 5, compounds with such a protein domain show a max. killing improvement factor ≥ 1.7 compared to ΔB7-H6_wt.)
The compound according to any one of items [1] to [87], wherein said protein domain of (a) consists of an amino acid sequence selected from the group consisting of SEQ ID NO: 17, 18, 19, 21, 24, 27, 28, 29, 30, 32 and 34. (As can be seen e.g. from Example 6, compounds with such a protein domain show an IFNγ release improvement factor > 1 compared to ΔB7-H6_wt.)
The compound according to any one of items [1] to [89], wherein said protein domain of (a) consists of an amino acid sequence selected from the group consisting of SEQ ID NO: 17, 18, 19, 21, 24, 27, 28, 29, 30, 32 and 34. (As can be seen e.g. from Example 6, compounds with such a protein domain show a TNFα release improvement factor > 1 compared to ΔB7-H6_wt.)
The compound according to any one of items [1] to [90], wherein said protein domain of (a) consists of an amino acid sequence selected from the group consisting of SEQ ID NO: 18, 19, 21, 24, 27, 28, 30, 32 and 34. (As can be seen e.g. from Example 6, compounds with such a protein domain show a TNFα release improvement factor > 8 compared to ΔB7-H6_wt.)
The compound according to any one of items [1] to [91], wherein said protein domain of (a) consists of an amino acid sequence selected from the group consisting of SEQ ID NO: 18, 21, 27, 30 and 34. (As can be seen e.g. from Example 6, compounds with such a protein domain show a TNFα release improvement factor > 10 compared to ΔB7-H6_wt.)
The compound according to any one of items [1] to [92], wherein said compound comprises or is a protein.
The compound according to any one of items [1] to [93], wherein said compound is a protein.
The compound according to any one of items [1] to [94], wherein said compound comprises said protein domain of (a) or (b).
The compound according to any one of items [1] to [95], wherein said compound comprises said protein domain of (a) or (c).
The compound according to any one of items [1] to [96], wherein said compound consists of said protein domain of (a), (b) or (c).
The compound according to any one of items [1] to [97], wherein said compound consists of said protein domain of (a) or (b).
The compound according to any one of items [1] to [98], wherein said compound consists of said protein domain of (a) or (c).
The compound according to any one of items [1] to [99], wherein said compound further comprises a targeting moiety.
The compound according to any one of items [1] to [100], wherein said compound comprises
   said protein domain of (a), (b) or (c) and
   a targeting moiety.
The compound according to any one of items [1] to [101], wherein said compound consists of
   said protein domain of (a), (b) or (c) and
   a targeting moiety.
The compound according to any one of items [1] to [102], wherein said compound comprises
   said protein domain of (a) and
   a targeting moiety.
The compound according to any one of items [1] to [103], wherein said compound consists of
   said protein domain of (a) and
   a targeting moiety.
The compound according to any one of items [1] to [104], wherein all components of said compound are covalently linked.
The compound according to any one of items [100] to [105], wherein said targeting moiety is a molecular group that specifically binds to a target molecule or fragment thereof.
The compound according to item [106], wherein said target molecule is a receptor at the surface of a cell.
The compound according to any one of items [106] to [107], wherein said target molecule is an antigen that is present on the surface of a target cell.
The compound according to any one of items [100] to [108], wherein said targeting moiety is capable of specifically binding to an antigen that is present on the surface of a target cell.
The compound according to any one of items [100] to [109], wherein said targeting moiety comprises a protein, a peptide, a peptide mimetic, a nucleic acid, an oligonucleotide or a small molecule.
The compound according to any one of items [100] to [110], wherein said targeting moiety is a protein, a peptide, a peptide mimetic, a nucleic acid, an oligonucleotide or a small molecule.
The compound according to any one of items [100] to [111], wherein said targeting moiety comprises a protein.
The compound according to any one of items [100] to [112], wherein said targeting moiety is a protein.
The compound according to any one of items [100] to [113], wherein said targeting moiety comprises or is a protein which is a protein ligand that specifically binds to a receptor at the surface of a cell.
The compound according to any one of items [100] to [114], wherein said targeting moiety comprises or is a protein which is an antibody or an antigen-binding fragment thereof.
The compound according to any one of items [100] to [115], wherein said targeting moiety comprises or is a protein which comprises at least 30 amino acids.
The compound according to any one of items [100] to [111], wherein said targeting moiety comprises or is a peptide which consists of 2 to 30 amino acids.
The compound according to any one of items [100] to [111] or [117], wherein said targeting moiety comprises or is a peptide which consists of 10 to 30 amino acids.
The compound according to any one of items [100] to [111] or [117] to [118], wherein said targeting moiety comprises a peptide.
The compound according to any one of items [100] to [111] or [117] to [119], wherein said targeting moiety is a peptide.
The compound according to any one of items [100] to [111], wherein said targeting moiety comprises a peptide mimetic.
The compound according to any one of items [100] to [111] or [121], wherein said targeting moiety is a peptide mimetic.
The compound according to any one of items [100] to [111], wherein said targeting moiety comprises or is a nucleic acid which is a DNA or an RNA.
The compound according to any one of items [100] to [111] or [123], wherein said targeting moiety comprises a nucleic acid.
The compound according to any one of items [100] to [111] or [123] to [124], wherein said targeting moiety is a nucleic acid.
The compound according to any one of items [100] to [111] or [123], wherein said targeting moiety comprises an oligonucleotide.
The compound according to any one of items [100] to [111] or [123] or [126], wherein said targeting moiety is an oligonucleotide.
The compound according to any one of items [100] to [111], wherein said targeting moiety comprises or is a small molecule with a molecular weight < 1000 Da.
The compound according to any one of items [100] to [111] or [128], wherein said targeting moiety comprises a small molecule.
The compound according to any one of items [100] to [111] or [128] to [129], wherein said targeting moiety is a small molecule.
The compound according to any one of items [100] to [130], wherein said targeting moiety has a molecular weight of at least 100 Da.
The compound according to any one of items [100] to [131], wherein said targeting moiety has a molecular weight of at least 500 Da.
The compound according to any one of items [100] to [132], wherein said targeting moiety has a molecular weight of at least 1 000 Da.
The compound according to any one of items [100] to [129] or [131] to [133], wherein said targeting moiety has a molecular weight of at least 2 000 Da.
The compound according to any one of items [100] to [119] or [121] to [126] or [128] to [129] or [131] to [134], wherein said targeting moiety has a molecular weight of at least 10 kDa.
The compound according to any one of items [100] to [119] or [121] to [126] or [128] to [129] or [131] to [135], wherein said targeting moiety has a molecular weight of at least 50 kDa.
The compound according to any one of items [100] to [119] or [121] to [126] or [128] to [129] or [131] to [136], wherein said targeting moiety has a molecular weight of at least 100 kDa.
The compound according to any one of items [100] to [137], wherein said targeting moiety has a molecular weight of up to 1 000 Da.
The compound according to any one of items [100] to [138], wherein said targeting moiety has a molecular weight of up to 2 000 Da.
The compound according to any one of items [100] to [139], wherein said targeting moiety has a molecular weight of up to 10 kDa.
The compound according to any one of items [100] to [140], wherein said targeting moiety has a molecular weight of up to 50 kDa.
The compound according to any one of items [100] to [141], wherein said targeting moiety has a molecular weight of up to 200 kDa.
The compound according to any one of items [100] to [142], wherein said targeting moiety has a molecular weight of up to 1 MDa.
The compound according to any one of items [100] to [143], wherein said targeting moiety has a molecular weight of up to 5 MDa.
The compound according to any one of items [100] to [144], wherein said targeting moiety has a molecular weight of up to 10 MDa.
The compound according to any one of items [100] to [145], wherein said targeting moiety is capable of specifically binding to a tumor-associated antigen or to an immune cell antigen.
The compound according to any one of items [100] to [146], wherein said targeting moiety is capable of specifically binding to a tumor-associated antigen.
The compound according to any one of items [100] to [119] or [121] or [123] to [124] or [126] or [128] to [129] or [131] to [147], wherein said targeting moiety comprises an antibody or antigen-binding fragment of an antibody.
The compound according to any one of items [100] to [119] or [121] or [123] to [124] or [126] or [128] to [129] or [131] to [148], wherein said targeting moiety is an antibody or antigen-binding fragment of an antibody.
The compound according to any one of items [100] to [119] or [121] or [123] to [124] or [126] or [128] to [129] or [131] to [149], wherein said targeting moiety is an antibody.
The compound according to any one of items [115] to [116] or [131] to [150], wherein said antibody is an intact antibody.
The compound according to any one of items [115] to [116] or [131] to [151], wherein said antibody is a monoclonal antibody or a polyclonal antibody.
The compound according to any one of items [115] to [116] or [131] to [152], wherein said antibody is a monoclonal antibody.
The compound according to any one of items [115] to [116] or [131] to [153], wherein said antibody is a monospecific antibody or a bispecific antibody.
The compound according to any one of items [115] to [116] or [131] to [154], wherein said antibody is a bispecific antibody.
The compound according to any one of items [115] to [116] or [131] to [154], wherein said is antibody monovalent.
The compound according to any one of items [115] to [116] or [131] to [155], wherein said antibody is bivalent.
The compound according to any one of items [115] to [116] or [131] to [155], wherein said antibody is multivalent.
The compound according to any one of items [115] to [116] or [131] to [158], wherein said antibody is an antibody selected from the group consisting of a chimeric antibody, a humanized antibody and a human antibody.
The compound according to any one of items [115] to [116] or [131] to [159], wherein said antibody is a human antibody.
The compound according to any one of items [115] to [116] or [131] to [160], wherein said antibody is an antibody selected from the group consisting of an IgG antibody, an IgA antibody, an IgM antibody, and hybrids thereof.
The compound according to any one of items [115] to [116] or [131] to [161], wherein said antibody is an antibody selected from the group consisting of an IgG antibody, an IgA antibody and hybrids thereof.
The compound according to any one of items [115] to [116] or [131] to [162], wherein said antibody is an IgG antibody.
The compound according to any one of items [161] to [163], wherein said IgG antibody is an IgG1 antibody, an IgG2 antibody, an IgG3 antibody or an IgG4 antibody.
The compound according to any one of items [161] to [164], wherein said IgG antibody is an IgG1 antibody.
The compound according to any one of items [115] to [116] or [131] to [165], wherein said antibody is an antibody prepared by the SEED (strand-exchange engineered domain) technology.
The compound according to any one of items [100] to [119] or [121] or [123] to [124] or [126] or [128] to [129] or [131] to [149], wherein said targeting moiety is an antigen-binding fragment of an antibody.
The compound according to any one of items [115] to [116] or [131] to [149] or [167], wherein said antigen-binding fragment is selected from the group consisting of a Fab, a Fab′, a (Fab′)2, a Fv, a scFv, a diabody and a VHH.
The compound according to any one of items [115] to [116] or [131] to [149] or [167] to [168], wherein said antigen-binding fragment is selected from the group consisting of a Fab, a Fab′, a (Fab′)2 and a Fv.
The compound according to any one of items [115] to [116] or [131] to [149] or [167] to [169], wherein said antigen-binding fragment is a Fab.
The compound according to any one of items [115] to [116] or [131] to [149] or [167] to [168], wherein said antigen-binding fragment is selected from the group consisting of a scFv, a diabody and a VHH.
The compound according to any one of items [115] to [116] or [131] to [149] or [167] to [171], wherein said antigen-binding fragment is an antigen-binding fragment of a monoclonal antibody or a polyclonal antibody.
The compound according to any one of items [115] to [116] or [131] to [149] or [167] to [172], wherein said antigen-binding fragment is an antigen-binding fragment of a monoclonal antibody.
The compound according to any one of items [115] to [116] or [131] to [149] or [167] to [173], wherein said antigen-binding fragment is an antigen-binding fragment of a monospecific antibody or a bispecific antibody.
The compound according to any one of items [115] to [116] or [131] to [149] or [167] to [174], wherein said antigen-binding fragment is an antigen-binding fragment of a bispecific antibody, and wherein said antigen-binding fragment is capable of binding both antigens for which said bispecific antibody is specific.
The compound according to any one of items [115] to [116] or [131] to [149] or [167] to [175], wherein said antigen-binding fragment is an antigen-binding fragment of an antibody selected from the group consisting of a chimeric antibody, a humanized antibody and a human antibody.
The compound according to any one of items [115] to [116] or [131] to [149] or [167] to [176], wherein said antigen-binding fragment is an antigen-binding fragment of a human antibody.
The compound according to any one of items [115] to [116] or [131] to [149] or [167] to [177], wherein said antigen-binding fragment is an antigen-binding fragment of an antibody selected from the group consisting of an IgG antibody, an IgA antibody, an IgM antibody, and hybrids thereof.
The compound according to any one of items [115] to [116] or [131] to [149] or [167] to [178], wherein said antigen-binding fragment is an antigen-binding fragment of an antibody selected from the group consisting of an IgG antibody, an IgA antibody and hybrids thereof.
The compound according to any one of items [115] to [116] or [131] to [149] or [167] to [179], wherein said antigen-binding fragment is an antigen-binding fragment of an IgG antibody.
The compound according to any one of items [178] to [180], wherein said IgG antibody is selected from the group consisting of an IgG1 antibody, an IgG2 antibody, an IgG3 antibody, IgG4 antibody and hybrids thereof.
The compound according to any one of items [178] to [181], wherein said IgG antibody is an IgG1 antibody.
The compound according to any one of items [115] to [116] or [131] to [149] or [167] to [182], wherein said antigen-binding fragment is an antigen-binding fragment of an antibody with the SEED (strand-exchange engineered domain) format.
The compound according to any one of items [100] to [183], wherein said targeting moiety is capable of specifically binding to an antigen that is present on the surface of a target cell.
The compound according to any one of items [115] to [116] or [131] to [184], wherein said antibody or antigen-binding fragment is capable of specifically binding to an antigen that is present on the surface of a target cell.
The compound according to any one of items [115] to [116] or [131] to [166] or [184] to [185], wherein said antibody is an antibody against an antigen that is present on the surface of a target cell.
The compound according to any one of items [115] to [116] or [131] to [149] or [167] to [185], wherein said antigen-binding fragment is an antigen-binding fragment of an antibody against an antigen that is present on the surface of a target cell.
The compound according to any one of items [108] to [116] or [131] to [187], wherein said antigen that is present on the surface of said target cell is more abundant on the surface of said target cell than on the surface of other cell types.
The compound according to any one of items [108] to [116] or [131] to [188], wherein said antigen that is present on the surface of said target cell is present on the surface of said target cell, but substantially not on the surface of other cell types.
The compound according to any one of items [108] to [116] or [131] to [189], wherein said antigen that is present on the surface of said target cell is present on the surface of said target cell, but not on the surface of other cell types.
The compound according to any one of items [108] to [116] or [131] to [190], wherein said binding of said targeting moiety to said antigen that is present on the surface of said target cell allows to recruit the compound specifically to said target cell.
The compound according to any one of items [108] to [116] or [131] to [166] or [184] to [191], wherein said binding of said antibody to said antigen that is present on the surface of said target cell allows to recruit the compound specifically to said target cell.
The compound according to any one of items [108] to [116] or [131] to [149] or [167] to [190], wherein said binding of said antigen-binding fragment to said antigen that is present on the surface of said target cell allows to recruit the compound specifically to said target cell.
The compound according to any one of items [108] to [116] or [131] to [193], wherein said antigen that is present on the surface of said target cell is a tumor-associated antigen or an immune cell antigen.
The compound according to any one of items [108] to [116] or [131] to [194], wherein said antigen that is present on the surface of said target cell is a tumor-associated antigen.
The compound according to any one of items [100] to [195], wherein said targeting moiety is capable of specifically binding to a tumor-associated antigen or to an immune cell antigen.
The compound according to any one of items [100] to [196], wherein said targeting moiety is capable of specifically binding to a tumor-associated antigen.
The compound according to any one of items [115] to [116] or [131] to [197], wherein said antibody or antigen-binding fragment is capable of specifically binding to a tumor-associated antigen or to an immune cell antigen.
The compound according to any one of items [115] to [116] or [131] to [198], wherein said antibody or antigen-binding fragment is capable of specifically binding to a tumor-associated antigen.
The compound according to any one of items [146] to [199], wherein said tumor-associated antigen is an antigen that is present on the surface of a tumor cell.
The compound according to any one of items [146] to [200],wherein said tumor-associated antigen is selected from the group consisting of CD1 Ia, CD4, CD19, CD20, CD21, CD22, CD23, CD25, CD52, CD30, CD33, CD37, CD40L, CD52, CD56, CD70, CD72, CD74, CD79a, CD79b, CD138, CD163, HER2, Her3, EGFR, Mucl8, integrin, PSMA, CEA, BLys, ROR1, NaPi2b, NaPi3b, CEACAM5, Muc1, integrin avb6, Met, Trop2, BCMA, disialoganglioside GD2, B-PR1B, E16, STEAP1, 0772P, Sema 5b, ETBR, MSG783, STEAP2, Trp4, CRIPTO, FcRH1, FcRH2, NCA, IL20R-alpha, Brevican, EphB2R, ASLG659, PSCA, GEDA, BAFF-R, CXCR5, HLA-DOB, P2X5, LY64, IRTA2, TENB2, PSMA, FOLH1, STR5, SSTR1, SSTR2, SSTR3, SSTR4, TGAV, ITGB6, CA9, EGFRvlll, IL2RA, AXL, CD3Q, TNFRSF8, TNFRSF17, CTAGs, CTA; CD174/Fucosyltransferase 3 (Lewis Blood Group), CLEC14A, GRP78, HSPA5, ASG-5, ENPP3, PRR4, GCC, GUCY2C, Liv-1, SLC39A8, 5T4, NCMA1, CanAg, FOLR1, GPN B, TIM-1, HAVCR1, Mindin/RG-1, B7-H4, VTCN1, PTK7, SDC1, a claudin (preferably claudin 18.2), RON, MST1 R, EPHA2, MS4A1, TNC (Tenascin C), FAP, DKK-1, CS1/SLAMF7, ENG (Endoglin), ANXA1 (Annexin A1), VCAM-1 (CD106) and folate receptor alpha.
The compound according to any one of items [146] to [201], wherein said tumor-associated antigen is selected from the group consisting of xCT, gpNMB, carbonic anhydrase IX (CAIX), cKIT, c-MET, Tumor-associated glycoprotein 72 (TAG-72), TROP-2, TRA-1-60, TRA, TNF- alpha, TM4SF1, TIM-1, TAA, TA-MUC1 (tumor-specific epitope of mucin-1), Sortilin (SORT1), STn, STING, STEAP-1, SSTR2, SSEA-4, SLITRK6, SLC44A4, SLAMF7, SAIL, Receptor tyrosine kinase (RTK), ROR2, ROR1, RNF43, Prolactin Receptor (PRLR), Polymorphic epithelial mucin (PEM), Phosphatidylserine (PS), Phosphatidyl Serine, PTK7, PSMA, PD-L1, P-Cadherin, OX001L, OAcGD2, Nectin-4, NaPi2b, NOTCH3, Mesothelin (MSLN), MUC16, MTX5, MTX3, MT1-MMP, MRC2, MET, MAGE, Ly6E, Lewis Y antigen, LRRC15, LRP-1, LIV-1, LHRH, LGR5, LGALS3BP, LAMP-1, KLK2, KAAG-1, IL4R, IL7R, IL1RAP, IL-4, IL-3, IL-2, IL-13R, IGF-1R, HSP90, HLA-DR, HER-3, HER-2, Globo H, GPR20, GPC3, GPC-1, GD3, GD2, GCC, FSH, FOLR-alpha, FOLR, FLT3, FGFR3, FGFR2, FCRH5, EphA3, EphA2, EpCAM, ETBR, ENPP3, EGFRviii, EGFR, EFNA4, Dysadherin, DR5 (Death receptor 5), DPEP3, DLL3, DLK-1, DCLK1, Cripto, Cathepsin D, CanAg, CXCR5, CSP-1, CLL-1, CLDN6, CLDN18.2, CEACAM6, CEACAM5, CEA, CDH6, CD79b, CD74, CD71, CD70, CD56, CD51, CD48, CD46, CD45, CD44v6, CD40L, CD38, CD37, CD352, CD33, CD317, CD30, CD300f, CD3, CD25, CD248, CD228, CD22, CD205, CD20, CD19, CD184, CD166, CD147, CD142, CD138, CD123, CCR7, CA9, CA6, C4.4a, BCMA, B7-H4, B7, -H3, Axl, ASCT2, AMHRII, ALK, AG-7, ADAM-9, 5T4, 4-1BB.
The compound according to any one of items [146] to [202], wherein said tumor-associated antigen is selected from the group consisting of EGFR (epidermal growth factor receptor), HER2 (Human Epidermal Growth Factor Receptor 2), PD-L1 (Programmed cell death 1 ligand 1) and CD20.
The compound according to any one of items [146] to [203], wherein said tumor-associated antigen is EGFR.
The compound according to any one of items [146] to [203], wherein said tumor-associated antigen is HER2.
The compound according to any one of items [146] to [203], wherein said tumor-associated antigen is PD-L1.
The compound according to any one of items [146] to [203], wherein said tumor-associated antigen is CD20.
The compound according to any one of items [115] to [116] or [131] to, wherein said antibody or antigen-binding fragment has a first and a second antigen-binding site.
The compound according to item [208], wherein said first antigen-binding site is capable of specifically binding to a tumor-associated antigen and said second antigen-binding site is capable of specifically binding to a tumor-associated antigen.
The compound according to any one of items [208] to [209], wherein said first and said second antigen-binding site are capable of binding to different antigens.
The compound according to any one of items [100] to [210], wherein said compound is a bispecific molecule that binds via the protein domain of (a)/(b)/(c) to NKp30 and via its targeting domain to a tumor-associated antigen.
The compound according to any one of items [1] to [211], wherein said compound is an antibody-drug conjugate (ADC) which comprises
   (i) a protein domain of (a)/(b)/(c),
   (ii) an antibody or antigen-binding fragment thereof,
   (iii) at least one payload wherein said at least one payload is a therapeutic agent.
The compound according to any one of items [1] to [212], wherein said compound does not comprise an antibody Fc region.
The compound according to any one of items [1] to [212], wherein said compound comprises an antibody Fc region.
The compound according to any one of items [1] to [214], wherein said compound does not comprise an antibody Fc region competent in Fc receptor binding.
The compound according to any one of items [1] to [212] or [214], wherein said compound comprises an antibody Fc region competent in Fc receptor binding.
The compound according to any one of items [1] to [216], wherein said compound does not comprise an antibody Fc region that is not competent in Fc receptor binding.
The compound according to any one of items [1] to [212] or [214] to [216], wherein said compound comprises an antibody Fc region that is not competent in Fc receptor binding.
The compound according to any one of items [1] to [218], wherein said compound does not comprise an effector-competent Fc region.
The compound according to any one of items [1] to [212] or [214] or [216] to [218], wherein said compound comprises an effector-competent Fc region.
The compound according to any one of items [1] to [220], wherein said compound does not comprise an antibody Fc region capable of inducing ADCC (antibody-dependent cellular cytotoxicity).
The compound according to any one of items [1] to [212] or [214] or [216] to [218] or [220], wherein said compound comprises an antibody Fc region capable of inducing ADCC.
The compound according to any one of items [1] to [222], wherein said compound does not comprise an antibody Fc region that is not capable of inducing ADCC.
The compound according to any one of items [214] or [216], wherein said Fc region is an Fc variant of a wild-type human IgG1 Fc region, wherein the Fc variant of the wild-type human IgG1 Fc region contains amino acid substitutions P329G, L234A and L235A (residues numbered according to the EU index of Kabat).
The compound according to any one of items [1] to [212] or [214] to [224], wherein said compound comprises an antibody Fc region that is not capable of inducing ADCC.
The compound according to any one of items [100] to [225], wherein said targeting moiety does not comprise an antibody Fc region.
The compound according to any one of items [100] to [225], wherein said targeting moiety comprises an antibody Fc region.
The compound according to any one of items [100] to [227], wherein said targeting moiety does not comprise an antibody Fc region competent in Fc receptor binding.
The compound according to any one of items [100] to [225] or [227], wherein said targeting moiety comprises an antibody Fc region competent in Fc receptor binding.
The compound according to any one of items [100] to [229], wherein said targeting moiety does not comprise an antibody Fc region that is not competent in Fc receptor binding.
The compound according to any one of items [100] to [225] or [227] to [229] or, wherein said targeting moiety comprises an antibody Fc region that is not competent in Fc receptor binding.
The compound according to any one of items [100] to [231], wherein said targeting moiety does not comprise an effector-competent Fc region.
The compound according to any one of items [100] to [225] or [227] to [231], wherein said targeting moiety comprises an effector-competent Fc region.
The compound according to any one of items [100] to [233], wherein said targeting moiety does not comprise an antibody Fc region capable of inducing ADCC (antibody-dependent cellular cytotoxicity).
The compound according to any one of items [100] to [225] or [227] to [231] or [233], wherein said targeting moiety comprises an antibody Fc region capable of inducing ADCC.
The compound according to any one of items [100] to [235], wherein said targeting moiety does not comprise an antibody Fc region that is not capable of inducing ADCC.
The compound according to any one of items [227] to [232] or [234] or [236], wherein said Fc region is an Fc variant of a wild-type human IgG1 Fc region, wherein the Fc variant of the wild-type human IgG1 Fc region contains amino acid substitutions P329G, L234A and L235A (residues numbered according to the EU index of Kabat).
The compound according to any one of items [100] to [225] or [227] to [237], wherein said targeting moiety comprises an antibody Fc region that is not capable of inducing ADCC.
The compound according to any one of items [213] to [238], wherein said antibody Fc region is a human Fc region.
The compound according to any one of items [213] to [239], wherein said antibody Fc region is an IgG Fc region.
The compound according to any one of items [213] to [240], wherein said antibody Fc region is an IgG1 Fc region.
The compound according to any one of items [1] to [241], wherein said compound is capable of inducing both FcγRIIIa signalling and positive (i.e. NK cell activating) NKp30 signalling.
The compound according to any one of items [1] to [242], wherein said compound is capable of specifically binding to NKp30 on NK cells.
The compound according to any one of items [1] to [243], wherein said compound is capable of activating NK cells by binding to NKp30 on said NK cells.
The compound according to any one of items [243] to [244], wherein binding of said compound to NKp30 on NK cells activates said NK cells.
The compound according to any one of items [1] to [245], wherein said compound is an agonist of NKp30.
The compound according to any one of items [1] to [246], wherein binding of said compound to NKp30 on NK cells allows to recruit said NK cells to said compound or said compound to said NK cells.
The compound according to any one of items [1] to [247], wherein said compound binds to NKp30 with a higher affinity (i.e. lower KD) than a comparative molecule.
The compound according to any one of items [1] to [248], wherein said compound binds to NKp30 with higher Kon rate than a comparative molecule.
The compound according to any one of items [1] to [249], wherein said compound binds to NKp30 with lower Koff rate than a comparative molecule.
The compound according to any one of items [248] to [250], wherein said affinity/said Kon rate/said Koff rate is measured by kinetic measurements by biolayer interferometry at 25° C. and 1000 rpm, wherein said compound resp. comparative molecule (5 µg/mL in PBS) is loaded on a anti-human Fc biosensor for 5 min followed by 60 s rinsing the sensor with kinetics buffer (KB; PBS + 0.1% Tween-20 + 1% BSA), wherein association to human NKp30 in varying concentrations (15.6-1000 nM) is measured for 60 s followed by dissociation measurement for 180 s in KB and data is fitted using a 1:1 binding model.
The compound according to any one of items [1] to [251], wherein binding of said compound to NKp30 on NK cells activates said NK cells with greater efficiency than binding of a comparative molecule.
The compound according to item [252], wherein the efficiency of activation of NK cells is measured in an assay in which 20,000 A431 cells/well are seeded in 96-well V-bottom microtiter plates and incubated for 3 h prior addition of 100,000 NK cells/well (E:T ratio of 5:1), that had previously been treated with 100 U/ml recombinant human interleukin-2 overnight, the compound to be tested is then added at a final concentration of 85 nM, incubated for 24 h at 37° C., washed two times with PBS + 1% BSA, incubated with dead cell stain, anti-human CD56 and anti-human CD69 for 1 h on ice, washed, followed by measurement of activated NK cells based on a gating strategy involving Side scatter (SSC) vs. forward scatter (FCS) for identification of single NK cells, followed by dead cell staining and CD56 staining versus an unrelated channel for the identification of living CD56⁺ NK cells followed by gating on activated CD56⁺ CD69⁺ NK cells, wherein a compound to be tested is considered to activate NK cells if the number of activated NK cells is increased in the presence of said compound to be tested.
The compound according to any one of items [1] to [253], wherein in a ⁵¹Cr release assay said compound has improved cytotoxic activity compared to a comparative molecule.
The compound according to item [254], wherein said ⁵¹Cr release assay is carried out as a 4 h ⁵¹Cr release assay, wherein human PBMCs are used as effector cells at effector-to-target cell (E:T) ratios of 80:1, and wherein a higher percent lysis indicates improved cytotoxic activity.
The compound according to item [255], wherein said ⁵¹Cr release assay is carried out as a 4 h ⁵¹Cr release assay, wherein human purified NK cells are used as effector cells at effector-to-target cell (E:T) ratios of 10:1, and wherein a higher percent lysis indicates improved cytotoxic activity.
The compound according to any one of items [254] to [256], wherein said ⁵¹Cr release assay is carried out as described in Repp et al., 2011.
The compound according to any one of items [1] to [257], wherein binding of said compound to NKp30 on NK cells results in a stronger release of interferon-γ (IFN-γ) than binding of a comparative molecule.
The compound according to item [258], wherein said release of IFN-γ is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human IFN-γ is analyzed in the culture supernatant after 24 h by ELISA.
The compound according to any one of items [1] to [259], wherein binding of said compound to NKp30 on NK cells results in a stronger release of tumor necrosis factor-α (TNF-α) than binding of a comparative molecule.
The compound according to item [260], wherein said release of TNF-α is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human TNF-α is analyzed in the culture supernatant after 24 h by ELISA.
The compound according to any one of items [248] to [261], wherein said comparative molecule is identical to said compound with the exception that in said comparative molecule
   X₁ is S;
   X₂ is G;
   X₃ is F;
   X₄ is G;
   X₅ is V;
   X₆ is T;
   X₇ is L;
   X₈ is K.
The compound according to any one of items [248] to [262], wherein said comparative molecule is identical to said compound with the exception that said comparative molecule does not comprise said protein domain of (a), (b) or (c), but instead comprises a protein domain with the amino acid sequence of SEQ ID NO: 2.
A pharmaceutical composition comprising the compound according to any one of items [1] to [263].
The pharmaceutical composition according to item [264], wherein said pharmaceutical composition comprises a pharmaceutically acceptable carrier, diluent and/or excipient.
The pharmaceutical composition according to any one of items [264] to [265], wherein said pharmaceutical composition further includes at least one additional adjuvant, antioxidant, buffering agent, bulking agent, colorant, emulsifier, filler, flavoring agent, preservative, stabilizer, suspending agent and/or other customary pharmaceutical auxiliary.
A compound according to any one of items [1] to [263] or the pharmaceutical composition according to any one of items [264] to [266] for use as a medicament.
A compound according to any one of items [1] to [263] or the pharmaceutical composition according to any one of items [264] to [266] for use in the treatment of cancer.
A compound according to any one of items [1] to [263] or the pharmaceutical composition according to any one of items [264] to [266] for use in the treatment of a malignant tumor.
A compound according to any one of items [1] to [263] or the pharmaceutical composition according to any one of items [264] to [266] for use in the treatment of an inflammatory disease.
A compound according to any one of items [1] to [263] or the pharmaceutical composition according to any one of items [264] to [266], wherein said compound/said pharmaceutical composition is for use in the treatment of a human.
A method for treating a disease in a patient in need thereof, comprising the step of administering to said patient a therapeutically effective amount of the compound according to any one of items [1] to [263] or the pharmaceutical composition according to any one of items [264] to [266].
The method according to item [272], wherein said disease is cancer.
The method according to item [272], wherein said disease is a malignant tumor.
The method according to item [272], wherein said disease is an inflammatory disease.
The method according to any one of items [272] to [275], wherein said patient is a human.
Use of the compound according to any one of items [1] to [263] or the pharmaceutical composition according to any one of items [264] to [266] for the manufacture of a medicament.
Use of the compound according to any one of items [1] to [263] or the pharmaceutical composition according to any one of items [264] to [266] for the manufacture of a medicament for the treatment of cancer.
Use of the compound according to any one of items [1] to [263] or the pharmaceutical composition according to any one of items [264] to [266] for the manufacture of a medicament for the treatment of a malignant tumor.
Use of the compound according to any one of items [1] to [263] or the pharmaceutical composition according to any one of items [264] to [266] for the manufacture of a medicament for the treatment of an inflammatory disease.
The use according to any one of items [277] to [280], wherein said medicament is prepared for administration to a human.
The compound or the pharmaceutical composition for use according to item [270] or the method according to item [275] or the use according to item [280], wherein said inflammatory disease is an autoimmune disease.
The compound or the pharmaceutical composition for use according to any one of items [270] or [282] or the method according to any one of items [275] or [282] or the use according to any one of items [280] or [282], wherein said inflammatory disease is selected from the group consisting of inflammatory bowel disease (IBD), systemic lupus erythematosus (SLE), multiple sclerosis, rheumatoid arthritis, Sjogren’s syndrome and Hidradenitis suppurativa (HS).
The compound or the pharmaceutical composition for use according to item [268] or the method according to item [273] or the use according to item [278], wherein said cancer, malignant tumor or inflammatory disease is a human disease.
A method for preparing a compound with an increased affinity for NKp30 compared to a compound comprising a protein domain with the amino acid sequence of SEQ ID NO: 2:

DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPAS (SEQ ID N
O: 2),

wherein said method comprises preparing a compound comprising a variant of the protein domain with the amino acid sequence of SEQ ID NO: 2 in which at least one of the following amino acid replacements has been carried out compared to the sequence of SEQ ID NO: 2:
   replacement of X₁ by I, L, T, V, H, W, Y, E or Q;
   replacement of X₃ by W or Y;
   replacement of X₄ by D, A or Q;
   replacement of X₅ by I or F;
   replacement of X₇ by Y, F or V,
   wherein X₁, X₃, X₄, X₅ and X₇ refer to the following positions within the sequence of SEQ ID NO: 2:

DLKVEMMAGGTQITPLNDNVTIFCNIFYSQPLNITX₁ MGITWFWKSLTFD
KEVKVFEFX₃X₄ DHQEAFRPGAIVSPWRLKSGDASLRLPGIQLEEAGEYR
CEVX₅ VTPX₇ KAQGTVQLEVVASPAS.

The method according to item [285], wherein said method comprises preparing a compound comprising a variant of the protein domain with the amino acid sequence of SEQ ID NO: 2 in which at least two of the following amino acid replacements have been carried out compared to the sequence of SEQ ID NO: 2:
   replacement of X₁ by I, L, T, V, H, W, Y, E or Q;
   replacement of X₃ by W or Y;
   replacement of X₄ by D, A or Q;
   replacement of X₅ by I or F;
   X₇ is replaced by Y, F or V.
The method according to any one of items [285] to [286], wherein said method comprises preparing a compound comprising a variant of the protein domain with the amino acid sequence of SEQ ID NO: 2 in which at least three of the following amino acid replacements have been carried out compared to the sequence of SEQ ID NO: 2:
   replacement of X₁ by I, L, T, V, H, W, Y, E or Q;
   replacement of X₃ by W or Y;
   replacement of X₄ by D, A or Q;
   replacement of X₅ by I or F;
   replacement of X₇ by Y, F or V.
The method according to any one items [285] to [287], wherein the method comprises preparing a compound comprising a variant of the protein domain with the amino acid sequence of SEQ ID NO: 2 in which outside of the positions X₁, X₃, X₄, X₅ and X₇ up to 24 amino acids have been added, replaced or deleted compared to the sequence of SEQ ID NO: 2.
The method according to any one items [285] to [288], wherein the method comprises preparing a compound comprising a variant of the protein domain with the amino acid sequence of SEQ ID NO: 2 in which outside of the positions X₁, X₃, X₄, X₅ and X₇ up to 18 amino acids have been added, replaced or deleted compared to the sequence of SEQ ID NO: 2:
The method according to any one items [285] to [289], wherein the method comprises preparing a compound comprising a variant of the protein domain with the amino acid sequence of SEQ ID NO: 2 in which outside of the positions X₁, X₃, X₄, X₅ and X₇ up to 12 amino acids have been added, replaced or deleted compared to the sequence of SEQ ID NO: 2.
The method according to any one items [285] to [290], wherein the method comprises preparing a compound comprising a variant of the protein domain with the amino acid sequence of SEQ ID NO: 2 in which outside of the positions X₁, X₃, X₄, X₅ and X₇ up to 6 amino acids have been added, replaced or deleted compared to the sequence of SEQ ID NO: 2.
The method according to any one items [285] to [291], wherein the method comprises preparing a compound comprising a variant of the protein domain with the amino acid sequence of SEQ ID NO: 2 in which outside of the positions X₁, X₃, X₄, X₅ and X₇ up to 5 amino acids have been added, replaced or deleted compared to the sequence of SEQ ID NO: 2.
The method according to any one items [285] to [292], wherein the method comprises preparing a compound comprising a variant of the protein domain with the amino acid sequence of SEQ ID NO: 2 in which outside of the positions X₁, X₃, X₄, X₅ and X₇ up to 4 amino acids have been added, replaced or deleted compared to the sequence of SEQ ID NO: 2.
The method according to any one items [285] to [293], wherein the method comprises preparing a compound comprising a variant of the protein domain with the amino acid sequence of SEQ ID NO: 2 in which outside of the positions X₁, X₃, X₄, X₅ and X₇ up to 3 amino acids have been added, replaced or deleted compared to the sequence of SEQ ID NO: 2.
The method according to any one items [285] to [294], wherein the method comprises preparing a compound comprising a variant of the protein domain with the amino acid sequence of SEQ ID NO: 2 in which outside of the positions X₁, X₃, X₄, X₅ and X₇ up to 2 amino acids have been added, replaced or deleted compared to the sequence of SEQ ID NO: 2.
The method according to any one items [285] to [295], wherein the method comprises preparing a compound comprising a variant of the protein domain with the amino acid sequence of SEQ ID NO: 2 in which outside of the positions X₁, X₃, X₄, X₅ and X₇ up to 1 amino acid has been added, replaced or deleted compared to the sequence of SEQ ID NO: 2.
The method according to any one items [285] to [296], wherein the method comprises preparing a compound comprising a variant of the protein domain with the amino acid sequence of SEQ ID NO: 2 in which outside of the positions X₁, X₃, X₄, X₅ and X₇ no amino acid has been added, replaced or deleted compared to the sequence of SEQ ID NO: 2.
The method according to any one items [285] to [297], wherein the method comprises preparing a compound comprising a variant of the protein domain with the amino acid sequence of SEQ ID NO: 2 in which the following two amino acids have not been replaced compared to the sequence of SEQ ID NO: 2:
   X₆;
   X₈,
   wherein X₆ and X₈ refer to the following positions within the sequence of SEQ ID NO: 2:

DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVX₆ PLX₈ AQGTVQLEVVASPAS.

The method according to any one items [285] to [298], wherein the method comprises preparing a compound comprising a variant of the protein domain with the amino acid sequence of SEQ ID NO: 2 in which the following three amino acids have not been replaced compared to the sequence of SEQ ID NO: 2:
   X₂;
   X₆;
   X₈,
   wherein X₂, X₆ and X₈ refer to the following positions within the sequence of SEQ ID NO: 2:

DLKVEMMAGGTQITPLNDNVTIFCNIFYSQPLNITSMX₂ ITWFWKSLTFD
KEVKVFEFFGDHQEAFRPGAIVSPWRLKSGDASLRLPGIQLEEAGEYRCE
VVVX₆ PLX₈ AQGTVQLEVVASPAS.

The method according to any one of items [288] to [299], wherein an amino acid has been added if said amino acid is present in the amino acid sequence of said variant of the protein domain with the amino acid sequence of SEQ ID NO: 2, but absent at the corresponding position in SEQ ID NO: 2 (wherein positions N-terminally of the first amino acid of SEQ ID NO: 2 or C-terminally of the last amino acid of SEQ ID NO: 2 are not considered).
The method according to any one of items [288] to [300], wherein an amino acid has been replaced by another amino acid if said amino acid is present in SEQ ID NO: 2, but absent at the corresponding position in the amino acid sequence of said variant of the protein domain with the amino acid sequence of SEQ ID NO: 2, and instead another amino acid is at the respective position present in the amino acid sequence of said variant.
The method according to any one of items [288] to [301], wherein an amino acid has been deleted if said amino acid is present in SEQ ID NO: 2, but absent at the corresponding position of the amino acid sequence of said variant of the protein domain with the amino acid sequence of SEQ ID NO: 2.
The method according to any one of items [285] to [302], wherein outside of said amino acid sequence of SEQ ID NO: 2 said compound comprising a protein domain with the amino acid sequence of SEQ ID NO: 2 is identical to said compound comprising a variant of the protein domain with the amino acid sequence of SEQ ID NO: 2.
The method according to any one of items [285] to [303], wherein said compound comprising a protein domain with the amino acid sequence of SEQ ID NO: 2 is a protein consisting of said protein domain with the amino acid sequence of SEQ ID NO: 2.
The method according to any one of items [285] to [304], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of SEQ ID NO: 4 to 15 or 17 to 50:

| | | | | | | | | |
|---|---|---|---|---|---|---|---|---|
| | X₁ | X₂ | X₃ | X₄ | X₅ | X₆ | X₇ | X₈ |
| SEQ ID NO: 4 | T | G | W | G | V | T | Y | K |
| SEQ ID NO: 5 | W | G | W | G | V | T | Y | K |
| SEQ ID NO: 6 | H | G | F | G | F | T | Y | K |
| SEQ ID NO: 7 | I | G | W | G | V | T | L | K |
| SEQ ID NO: 8 | V | G | W | G | V | T | Y | K |
| SEQ ID NO: 9 | H | G | W | G | V | T | Y | K |
| SEQ ID NO: 10 | H | G | Y | G | V | T | Y | K |
| SEQ ID NO: 11 | H | G | W | G | I | T | L | K |
| SEQ ID NO: 12 | H | G | F | G | V | T | Y | K |
| SEQ ID NO: 13 | S | G | Y | G | F | T | Y | K |
| SEQ ID NO: 14 | L | G | W | G | I | T | F | K |
| SEQ ID NO: 15 | E | G | F | G | V | T | Y | K |
| SEQ ID NO: 17 | T | G | Y | G | V | T | Y | K |
| SEQ ID NO: 18 | I | G | W | G | V | T | Y | K |
| SEQ ID NO: 19 | L | G | Y | G | V | T | Y | K |
| SEQ ID NO: 20 | S | G | Y | G | F | T | L | K |
| SEQ ID NO: 21 | Y | G | W | G | V | T | Y | K |
| SEQ ID NO: 22 | Q | G | W | G | V | T | Y | K |
| SEQ ID NO: 23 | H | G | W | G | F | T | L | K |
| SEQ ID NO: 24 | L | G | W | G | V | T | Y | K |
| SEQ ID NO: 25 | E | G | W | G | V | T | Y | K |
| SEQ ID NO: 26 | E | G | Y | G | V | T | Y | K |
| SEQ ID NO: 27 | I | G | Y | G | V | T | Y | K |
| SEQ ID NO: 28 | W | G | Y | G | V | T | Y | K |
| SEQ ID NO: 29 | T | G | W | D | V | T | Y | K |
| SEQ ID NO: 30 | T | G | W | G | I | T | Y | K |
| SEQ ID NO: 31 | H | G | W | G | V | T | L | K |
| SEQ ID NO: 32 | V | G | W | G | I | T | Y | K |
| SEQ ID NO: 33 | E | G | W | G | I | T | L | K |
| SEQ ID NO: 34 | I | G | Y | G | V | T | L | K |
| SEQ ID NO: 35 | E | G | W | G | V | T | V | K |
| SEQ ID NO: 36 | S | G | Y | G | V | T | Y | K |
| SEQ ID NO: 37 | S | G | W | D | V | T | Y | K |
| SEQ ID NO: 38 | T | G | W | G | V | T | L | K |
| SEQ ID NO: 39 | H | G | Y | A | V | T | Y | K |
| SEQ ID NO: 40 | S | G | W | G | F | T | Y | K |
| SEQ ID NO: 41 | E | G | F | G | I | T | L | K |
| SEQ ID NO: 42 | S | G | Y | G | I | T | Y | K |
| SEQ ID NO: 43 | T | G | W | G | V | T | V | K |
| SEQ ID NO: 44 | S | G | Y | G | V | T | L | K |
| SEQ ID NO: 45 | L | G | W | G | V | T | L | K |
| SEQ ID NO: 46 | T | G | W | Q | V | T | L | K |
| SEQ ID NO: 47 | H | G | Y | G | V | T | L | K |
| SEQ ID NO: 48 | S | G | W | G | V | T | Y | K |
| SEQ ID NO: 49 | T | G | F | G | V | T | Y | K |
| SEQ ID NO: 50 | E | G | Y | G | V | T | L | K |

wherein X₁ to X₈ refers to the following positions within the sequence of SEQ ID NO: 2:

DLKVEMMAGGTQITPLNDNVTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS.

The method according to any one of items [285] to [305], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 6, 8, 9, 10, 11, 12, 13, 14, 15, 17, 18, 19, 20, 21, 22, 24, 25, 26, 27, 28, 29, 30, 32, 33, 34, 36, 38, 42, 45 and 48.
The method according to any one of items [285] to [306], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 8, 9, 11, 12, 14, 17, 18, 19, 20, 21, 24, 27, 28, 32 and 34.
The method according to any one of items [285] to [307], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 8, 9, 14, 17, 18, 19, 21, 24, 27 and 32.
The method according to any one of items [385] to [308], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 8, 18, 21, 24, 27 and 32.
The method according to any one of items [285] to [309], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of SEQ ID NOs selected from the group consisting of SEQ ID NO: 18, 21, 24 and 27.
The method according to any one of items [285] to [310], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 8, 9, 10, 11, 12, 13, 14, 17, 18, 19, 21, 24, 26, 27, 28, 29, 30, 32 and 34.
The method according to any one of items [285] to [311], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 9, 11, 17, 18, 19, 21, 24, 27, 28, 29, 30, 32 and 34.
The method according to any one of items [285] to [312], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs from the group consisting of SEQ ID NO: 17, 18, 21, 27, 28, 30, 32 and 34.
The method according to any one of items [285] to [313], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18 and 32.
The method according to any one of items [285] to [314], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 6, 8, 9, 10, 11, 12, 13, 14, 15, 17, 18, 19, 21, 22, 24, 25, 26, 27, 28, 29, 30, 31, 33, 34, 36, 38, 42, 45, 46, 48, 49, 50.
The method according to any one of items [285] to [315], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 4, 6, 9, 10, 11, 12, 13, 15, 19, 21, 30, 31, 36, 42, 45, 48, 49.
The method according to any one of items [285] to [316], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 13, 19, 30 and 48.
The method according to any one of items [285] to [317], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 17, 18, 19, 21, 24, 27, 28, 29, 30, 32 and 34.
The method according to any one of items [285] to [318], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 19, 21 and 27.
The method according to any one of items [285] to [319], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 17, 18, 19, 21, 24, 27, 28, 29, 30, 32 and 34.
The method according to any one of items [285] to [320], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 19, 21, 24, 27, 28, 30, 32 and 34.
The method according to any one of items [285] to [321], wherein the method comprises preparing a compound comprising a variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of the SEQ ID NOs selected from the group consisting of SEQ ID NO: 18, 21, 27, 30 and 34.
The method according to any one of items [285] to [322], wherein said compound comprising a variant of the protein domain with the amino acid sequence of SEQ ID NO: 2 is capable of specifically binding to NKp30.
The method according to any one of items [285] to [323], wherein said compound comprising a variant of the protein domain with the amino acid sequence of SEQ ID NO: 2 is, upon binding to NKp30 on NK cells, capable of activating NK cells.
The method according to any one of items [285] to [324], wherein said compound comprising a variant of the protein domain with the amino acid sequence of SEQ ID NO: 2 is capable of specifically binding to NKp30 on NK cells.
The method according to any one of items [285] to [325], wherein said compound comprising a variant of the protein domain with the amino acid sequence of SEQ ID NO: 2 is capable of activating NK cells by binding to NKp30 on said NK cells.
The method according to any one of items [285] to [326], wherein binding of said compound comprising a variant of the protein domain with the amino acid sequence of SEQ ID NO: 2 to NKp30 on NK cells activates said NK cells.
The method according to any one of items [285] to [327], wherein said compound comprising a variant of the protein domain with the amino acid sequence of SEQ ID NO: 2 is an agonist of NKp30.
The method according to any one of items [285] to [328], wherein binding of said compound comprising a variant of the protein domain with the amino acid sequence of SEQ ID NO: 2 to NKp30 on NK cells allows to recruit said NK cells to said compound or said compound to said NK cells.
The method according to any one of items [285] to [329], wherein said compound comprising a variant of the protein domain with the amino acid sequence of SEQ ID NO: 2 binds to NKp30 with a higher Kon rate than said compound comprising a protein domain with the amino acid sequence of SEQ ID NO: 2.
The method according to any one of items [285] to [330], wherein said compound comprising a variant of the protein domain with the amino acid sequence of SEQ ID NO: 2 binds to NKp30 with a lower Koff rate than said compound comprising a protein domain with the amino acid sequence of SEQ ID NO: 2.
The method according to any one of items [285] to [331], wherein binding of said compound comprising a variant of the protein domain with the amino acid sequence of SEQ ID NO: 2 to NKp30 on NK cells activates said NK cells with greater efficiency than binding of said compound comprising a protein domain with the amino acid sequence of SEQ ID NO: 2.
The method according to item [332], wherein the efficiency of activation of NK cells is measured in an assay in which 20,000 A431 cells/well are seeded in 96-well V-bottom microtiter plates and incubated for 3 h prior addition of 100,000 NK cells/well (E:T ratio of 5:1), that had previously been treated with 100 U/ml recombinant human interleukin-2 overnight, the compound to be tested is then added at a final concentration of 85 nM, incubated for 24 h at 37° C., washed two times with PBS + 1% BSA, incubated with dead cell stain, anti-human CD56 and anti-human CD69 for 1 h on ice, washed, followed by measurement of activated NK cells based on a gating strategy involving Side scatter (SSC) vs. forward scatter (FCS) for identification of single NK cells, followed by dead cell staining and CD56 staining versus an unrelated channel for the identification of living CD56⁺ NK cells followed by gating on activated CD56⁺ CD69⁺ NK cells, wherein a compound to be tested is considered to activate NK cells if the number of activated NK cells is increased in the presence of said compound to be tested.
The method according to any one of items [285] to [333], wherein in a ⁵¹Cr release assay said compound comprising a variant of the protein domain with the amino acid sequence of SEQ ID NO: 2 has improved cytotoxic activity compared to said compound comprising a protein domain with the amino acid sequence of SEQ ID NO: 2.
The method according to item [334], wherein said ⁵¹Cr release assay is carried out as a 4 h ⁵¹Cr release assay, wherein human PBMCs are used as effector cells at effector-to-target cell (E:T) ratios of 80:1, and wherein a higher percent lysis indicates improved cytotoxic activity.
The method according to item [334], wherein said ⁵¹Cr release assay is carried out as a 4 h ⁵¹Cr release assay, wherein human purified NK cells are used as effector cells at effector-to-target cell (E:T) ratios of 10:1, and wherein a higher percent lysis indicates improved cytotoxic activity.
The method according to item [334] to [336], wherein said ⁵¹Cr release assay is carried out as described in Repp et al., 2011.
The method according to any one of items [285] to [337], wherein binding of said compound comprising a variant of the protein domain with the amino acid sequence of SEQ ID NO: 2 to NKp30 on NK cells results in a stronger release of interferon-y (IFN-γ) than binding of said compound comprising a protein domain with the amino acid sequence of SEQ ID NO: 2.
The method according to item [338], wherein said release of IFN-γ is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human IFN-γ is analyzed in the culture supernatant after 24 h by ELISA.
The method according to any one of items [285] to [339], wherein binding of said compound comprising a variant of the protein domain with the amino acid sequence of SEQ ID NO: 2 to NKp30 on NK cells results in a stronger release of tumor necrosis factor-α (TNF-α) than binding of said compound comprising a protein domain with the amino acid sequence of SEQ ID NO: 2.
The method according to item [340], wherein said release of TNF-α is measured as follows: isolated human NK cells are incubated overnight in medium containing 100 U/ml recombinant human interleukin-2, in different wells A431 cells are seeded and incubated for 3 h, immunoligands are added to a final concentration of 85 nM followed by addition of NK cells at an E:T ratio of 5:1, human TNF-α is analyzed in the culture supernatant after 24 h by ELISA.
The compound according to any one of items [1] to [263] or the pharmaceutical composition according to any one of items [264] to [266] or the compound or pharmaceutical composition for use according to any one of items [267] to [271] or [282] to [284] or the method according to any one of items [272] to [276] or [282] to [341] or the use according to any one of items [277] to [284], wherein said protein domain of (a) consists of an amino acid sequence selected from the group consisting of SEQ ID NO: 4, 5, 6, 7, 8, 9, 10, 11, 12, 13, 14, 15, 17, 18, 19, 20, 21, 22, 23, 24, 25, 26, 27, 28, 29, 30, 32, 33, 34, 35, 36, 38, 39, 42, 45 and 48. (As can be seen e.g. from Table 3 in Example 4, compounds with such a protein domain show a KD improvement factor ≥ 2 compared to ΔB7-H6_wt.)
The compound according to any one of items [1] to [263] or [342] or the pharmaceutical composition according to any one of items [264] to [266] or [342] or the compound or pharmaceutical composition for use according to any one of items [267] to [271] or [282] to [284] or [342] or the method according to any one of items [272] to [276] or [282] to [342] or the use according to any one of items [277] to [284] or [342], wherein said protein domain of (a) consists of an amino acid sequence selected from the group consisting of SEQ ID NO: 4, 5, 7, 8, 9, 11, 12, 14, 17, 18, 19, 20, 21, 23, 24, 27, 28, 32 and 34. (As can be seen e.g. from Table 3 in Example 4, compounds with such a protein domain show a KD improvement factor ≥ 5 compared to ΔB7-H6_wt.)
The compound according to any one of items [1] to [263] or [342] to [343] or the pharmaceutical composition according to any one of items [264] to [266] or [342] to [343] or the compound or pharmaceutical composition for use according to any one of items [267] to [271] or [282] to [284] or [342] to [343] or the method according to any one of items [272] to [276] or [282] to [343] or the use according to any one of items [277] to [284] or [342] to [343], wherein said protein domain of (a) consists of an amino acid sequence selected from the group consisting of SEQ ID NO: 4, 5, 7, 8, 9, 14, 17, 18, 19, 21, 24, 27 and 32. (As can be seen e.g. from Table 3 in Example 4, compounds with such a protein domain show a KD improvement factor ≥ 10 compared to ΔB7-H6_wt.)
The compound according to any one of items [1] to [263] or [342] to [344] or the pharmaceutical composition according to any one of items [264] to [266] or [342] to [344] or the compound or pharmaceutical composition for use according to any one of items [267] to [271] or [282] to [284] or [342] to [344] or the method according to any one of items [272] to [276] or [282] to [344] or the use according to any one of items [277] to [284] or [342] to [344], wherein said protein domain of (a) consists of an amino acid sequence selected from the group consisting of SEQ ID NO: 5, 8, 8, 18, 21, 24, 27 and 32. (As can be seen e.g. from Table 3 in Example 4, compounds with such a protein domain show a KD improvement factor ≥ 15 compared to ΔB7-H6_wt.)
The compound according to any one of items [1] to [263] or [342] to [345] or the pharmaceutical composition according to any one of items [264] to [266] or [342] to [345] or the compound or pharmaceutical composition for use according to any one of items [267] to [271] or [282] to [284] or [342] to [345] or the method according to any one of items [272] to [276] or [282] to [345] or the use according to any one of items [277] to [284] or [342] to [345], wherein said protein domain of (a) consists of an amino acid sequence selected from the group consisting of SEQ ID NO: 5, 18, 21, 24 and 27. (As can be seen e.g. from Table 3 in Example 4, compounds with such a protein domain show a KD improvement factor ≥ 20 compared to ΔB7-H6_wt.)
The compound according to any one of items [32] to [263] or [342] to [346] or the pharmaceutical composition according to any one of items [264] to [266] or [342] to [346] or the compound or pharmaceutical composition for use according to any one of items [267] to [271] or [282] to [284] or [342] to [346] or the method according to any one of items [272] to [276] or [282] to [346] or the use according to any one of items [277] to [284] or [342] to [346], wherein an amino acid is added if said amino acid is present in the amino acid sequence of said protein domain of (c), but said amino acid is absent at the corresponding position of said at least one sequence listed under (a).
The compound according to any one of items [32] to [263] or [342] to [347] or the pharmaceutical composition according to any one of items [264] to [266] or [342] to [347] or the compound or pharmaceutical composition for use according to any one of items [267] to [271] or [282] to [284] or [342] to [347] or the method according to any one of items [272] to [276] or [282] to [347] or the use according to any one of items [277] to [284] or [342] to [347], wherein an amino acid is replaced by another amino acid if said amino acid is present in said at least one sequence listed under (a), but absent at the corresponding position in the amino acid sequence of said protein domain of (c), and instead another amino acid is present in the amino acid sequence of said protein domain of (c) at the respective position.
The compound according to any one of items [32] to [263] or [342] to [348] or the pharmaceutical composition according to any one of items [264] to [266] or [342] to [348] or the compound or pharmaceutical composition for use according to any one of items [267] to [271] or [282] to [284] or [342] to [348] or the method according to any one of items [272] to [276] or [282] to [348] or the use according to any one of items [277] to [284] or [342] to [348], wherein said replacement is a conservative amino acid replacement.
The compound according to any one of items [32] to [263] or [342] to [349] or the pharmaceutical composition according to any one of items [264] to [266] or [342] to [349] or the compound or pharmaceutical composition for use according to any one of items [267] to [271] or [282] to [284] or [342] to [349] or the method according to any one of items [272] to [276] or [282] to [349] or the use according to any one of items [277] to [284] or [342] to [349], wherein an amino acid is deleted if said amino acid is present in said at least one sequence listed under (a), but absent at the corresponding position of the amino acid sequence of said protein domain of (c).
The compound according to any one of items [32] to [263] or [342] to [350] or the pharmaceutical composition according to any one of items [264] to [266] or [342] to [350] or the compound or pharmaceutical composition for use according to any one of items [267] to [271] or [282] to [284] or [342] to [350] or the method according to any one of items [272] to [276] or [282] to [350] or the use according to any one of items [277] to [284] or [342] to [350], wherein none of the amino acids shown in bold in said at least one sequence listed under (a) is replaced or deleted in the amino acid sequence of said protein domain of (c).
The compound according to any one of items [32] to [263] or [342] to [351] or the pharmaceutical composition according to any one of items [264] to [266] or [342] to [351] or the compound or pharmaceutical composition for use according to any one of items [267] to [271] or [282] to [284] or [342] to [351] or the method according to any one of items [272] to [276] or [282] to [351] or the use according to any one of items [277] to [284] or [342] to [351], wherein all amino acids depicted in bold in said at least one sequence listed under (a) are conserved in the amino acid sequence of said protein domain of (c).
The compound according to any one of items [32] to [263] or [342] to [352] or the pharmaceutical composition according to any one of items [264] to [266] or [342] to [352] or the compound or pharmaceutical composition for use according to any one of items [267] to [271] or [282] to [284] or [342] to [352] or the method according to any one of items [272] to [276] or [282] to [352] or the use according to any one of items [277] to [284] or [342] to [352], wherein said compound binds to NKp30 with a higher affinity compared to a corresponding compound in which said protein domain is replaced by SEQ ID NO: 2, reflected by a difference in the KD by a factor of ≥ 2 (i.e. the KD is smaller by a factor ≥ 2) compared to a corresponding compound in which said protein domain is replaced by SEQ ID NO: 2.
The compound according to any one of items [32] to [263] or [342] to [353] or the pharmaceutical composition according to any one of items [264] to [266] or [342] to [353] or the compound or pharmaceutical composition for use according to any one of items [267] to [271] or [282] to [284] or [342] to [353] or the method according to any one of items [272] to [276] or [282] to [353] or the use according to any one of items [277] to [284] or [342] to [353], wherein said compound binds to NKp30 with a higher affinity compared to a corresponding compound in which said protein domain is replaced by SEQ ID NO: 2, reflected by a difference in the KD by a factor of ≥ 5 (i.e. the KD is smaller by a factor ≥ 5) compared to a corresponding compound in which said protein domain is replaced by SEQ ID NO: 2.
The compound according to any one of items [32] to [263] or [342] to [354] or the pharmaceutical composition according to any one of items [264] to [266] or [342] to [354] or the compound or pharmaceutical composition for use according to any one of items [267] to [271] or [282] to [284] or [342] to [354] or the method according to any one of items [272] to [276] or [282] to [354] or the use according to any one of items [277] to [284] or [342] to [354], wherein said compound binds to NKp30 with a higher affinity compared to a corresponding compound in which said protein domain is replaced by SEQ ID NO: 2, reflected by a difference in the KD by a factor of ≥ 10 (i.e. the KD is smaller by a factor ≥ 10) compared to a corresponding compound in which said protein domain is replaced by SEQ ID NO: 2.
The compound according to any one of items [32] to [263] or [342] to [355] or the pharmaceutical composition according to any one of items [264] to [266] or [342] to [355] or the compound or pharmaceutical composition for use according to any one of items [267] to [271] or [282] to [284] or [342] to [355] or the method according to any one of items [272] to [276] or [282] to [355] or the use according to any one of items [277] to [284] or [342] to [355], wherein said compound binds to NKp30 with a higher affinity compared to a corresponding compound in which said protein domain is replaced by SEQ ID NO: 2, reflected by a difference in the KD by a factor of ≥ 15 (i.e. the KD is smaller by a factor ≥ 15) compared to a corresponding compound in which said protein domain is replaced by SEQ ID NO: 2.
The compound according to any one of items [32] to [263] or [342] to [356] or the pharmaceutical composition according to any one of items [264] to [266] or [342] to [356] or the compound or pharmaceutical composition for use according to any one of items [267] to [271] or [282] to [284] or [342] to [356] or the method according to any one of items [272] to [276] or [282] to [356] or the use according to any one of items [277] to [284] or [342] to [356], wherein said compound binds to NKp30 with a higher affinity compared to a corresponding compound in which said protein domain is replaced by SEQ ID NO: 2, reflected by a difference in the KD by a factor of ≥ 20 (i.e. the KD is smaller by a factor ≥ 20) compared to a corresponding compound in which said protein domain is replaced by SEQ ID NO: 2.
The compound according to any one of items [32] to [263] or [342] to [357] or the pharmaceutical composition according to any one of items [264] to [266] or [342] to [357] or the compound or pharmaceutical composition for use according to any one of items [267] to [271] or [282] to [284] or [342] to [357] or the method according to any one of items [272] to [276] or [282] to [357] or the use according to any one of items [277] to [284] or [342] to [357], wherein the half maximal effective concentration (EC50) of said compound for NK cell activation in the assay according to item [253] is by a factor of ≥ 10 lower compared to a corresponding compound in which said protein domain is replaced by SEQ ID NO: 2.
The compound according to any one of items [32] to [263] or [342] to [358] or the pharmaceutical composition according to any one of items [264] to [266] or [342] to [358] or the compound or pharmaceutical composition for use according to any one of items [267] to [271] or [282] to [284] or [342] to [358] or the method according to any one of items [272] to [276] or [282] to [358] or the use according to any one of items [277] to [284] or [342] to [358], wherein the half maximal effective concentration (EC50) of said compound for NK cell activation in the assay according to item [253] is by a factor of ≥ 20 lower compared to a corresponding compound in which said protein domain is replaced by SEQ ID NO: 2.
The compound according to any one of items [32] to [263] or [342] to [359] or the pharmaceutical composition according to any one of items [264] to [266] or [342] to [359] or the compound or pharmaceutical composition for use according to any one of items [267] to [271] or [282] to [284] or [342] to [359] or the method according to any one of items [272] to [276] or [282] to [359] or the use according to any one of items [277] to [284] or [342] to [359], wherein the half maximal effective concentration (EC50) of said compound for NK cell activation in the assay according to item [253] is by a factor of ≥ 40 lower compared to a corresponding compound in which said protein domain is replaced by SEQ ID NO: 2.
The compound according to any one of items [32] to [263] or [342] to [360] or the pharmaceutical composition according to any one of items [264] to [266] or [342] to [360] or the compound or pharmaceutical composition for use according to any one of items [267] to [271] or [282] to [284] or [342] to [360] or the method according to any one of items [272] to [276] or [282] to [360] or the use according to any one of items [277] to [284] or [342] to [360], wherein the half maximal effective concentration (EC50) of said compound for NK cell activation in the assay according to item [253] is by a factor of ≥ 100 lower compared to a corresponding compound in which said protein domain is replaced by SEQ ID NO: 2.
The compound according to any one of items [32] to [263] or [342] to [361] or the pharmaceutical composition according to any one of items [264] to [266] or [342] to [361] or the compound or pharmaceutical composition for use according to any one of items [267] to [271] or [282] to [284] or [342] to [361] or the method according to any one of items [272] to [276] or [282] to [361] or the use according to any one of items [277] to [284] or [342] to [361], wherein the maximal killing efficiency of said compound is by a factor of ≥ 1.3 higher compared to a corresponding compound in which said protein domain is replaced by SEQ ID NO: 2, wherein the maximal killing efficiency is measured by a 4 h ⁵¹Cr release assay, wherein human PBMCs are used as effector cells at effector-to-target cell (E:T) ratios of 80:1, and wherein killing efficiency is measured as percent lysis.
The compound according to any one of items [32] to [263] or [342] to [362] or the pharmaceutical composition according to any one of items [264] to [266] or [342] to [362] or the compound or pharmaceutical composition for use according to any one of items [267] to [271] or [282] to [284] or [342] to [362] or the method according to any one of items [272] to [276] or [282] to [362] or the use according to any one of items [277] to [284] or [342] to [362], wherein the maximal killing efficiency of said compound is by a factor of ≥ 1.5 higher compared to a corresponding compound in which said protein domain is replaced by SEQ ID NO: 2, wherein the maximal killing efficiency is measured by a 4 h ⁵¹Cr release assay, wherein human PBMCs are used as effector cells at effector-to-target cell (E:T) ratios of 80:1, and wherein killing efficiency is measured as percent lysis.
The compound according to any one of items [32] to [263] or [342] to [363] or the pharmaceutical composition according to any one of items [264] to [266] or [342] to [363] or the compound or pharmaceutical composition for use according to any one of items [267] to [271] or [282] to [284] or [342] to [363] or the method according to any one of items [272] to [276] or [282] to [363] or the use according to any one of items [277] to [284] or [342] to [363], wherein the maximal killing efficiency of said compound is by a factor of ≥ 1.7 higher compared to a corresponding compound in which said protein domain is replaced by SEQ ID NO: 2, wherein the maximal killing efficiency is measured by a 4 h ⁵¹Cr release assay, wherein human PBMCs are used as effector cells at effector-to-target cell (E:T) ratios of 80:1, and wherein killing efficiency is measured as percent lysis.
The compound according to any one of items [32] to [263] or [342] to [364] or the pharmaceutical composition according to any one of items [264] to [266] or [342] to [364] or the compound or pharmaceutical composition for use according to any one of items [267] to [271] or [282] to [284] or [342] to [364] or the method according to any one of items [272] to [276] or [282] to [364] or the use according to any one of items [277] to [284] or [342] to [364], wherein binding of said compound to NKp30 on NK cells results in a release of interferon-y (IFN-γ) that is higher (i.e. by a factor of > 1 increased) compared to a corresponding compound in which said protein domain is replaced by SEQ ID NO: 2, as measured according to item [259].
The compound according to any one of items [32] to [263] or [342] to [365] or the pharmaceutical composition according to any one of items [264] to [266] or [342] to [365] or the compound or pharmaceutical composition for use according to any one of items [267] to [271] or [282] to [284] or [342] to [365] or the method according to any one of items [272] to [276] or [282] to [365] or the use according to any one of items [277] to [284] or [342] to [365], wherein binding of said compound to NKp30 on NK cells results in a release of interferon-y (IFN-γ) that is by a factor > 3 higher compared to a corresponding compound in which said protein domain is replaced by SEQ ID NO: 2, as measured according to item [259].
The compound according to any one of items [32] to [263] or [342] to [366] or the pharmaceutical composition according to any one of items [264] to [266] or [342] to [366] or the compound or pharmaceutical composition for use according to any one of items [267] to [271] or [282] to [284] or [342] to [366] or the method according to any one of items [272] to [276] or [282] to [366] or the use according to any one of items [277] to [284] or [342] to [366], wherein binding of said compound to NKp30 on NK cells results in a release of tumor necrosis factor-α (TNF-α) that is higher (i.e. by a factor of > 1 increased) compared to a corresponding compound in which said protein domain is replaced by SEQ ID NO: 2, as measured according to item [261].
The compound according to any one of items [32] to [263] or [342] to [367] or the pharmaceutical composition according to any one of items [264] to [266] or [342] to [367] or the compound or pharmaceutical composition for use according to any one of items [267] to [271] or [282] to [284] or [342] to [367] or the method according to any one of items [272] to [276] or [282] to [367] or the use according to any one of items [277] to [284] or [342] to [367], wherein binding of said compound to NKp30 on NK cells results in a release of tumor necrosis factor-α (TNF-α) that is by a factor > 8 higher compared to a corresponding compound in which said protein domain is replaced by SEQ ID NO: 2, as measured according to item [261].
The compound according to any one of items [32] to [263] or [342] to [368] or the pharmaceutical composition according to any one of items [264] to [266] or [342] to [368] or the compound or pharmaceutical composition for use according to any one of items [267] to [271] or [282] to [284] or [342] to [368] or the method according to any one of items [272] to [276] or [282] to [368] or the use according to any one of items [277] to [284] or [342] to [368], wherein binding of said compound to NKp30 on NK cells results in a release of tumor necrosis factor-α (TNF-α) that is by a factor > 10 higher compared to a corresponding compound in which said protein domain is replaced by SEQ ID NO: 2, as measured according to item [261].

### EXAMPLES

The following examples describe the preparation and characterization of NKp30 binders as disclosed in the present disclosure, as well as related compounds and methods, along with comparative disclosure. It is understood that various embodiments of the disclosure reflected in the examples may be practiced, given the general description provided above. Although the foregoing invention has been described in some detail by way of illustration and example for purposes of clarity of understanding, the description and examples should not be construed as limiting the scope of the invention.

### Example 1

### Yeast Surface Display and Affinity Maturation of ΔB7-H6 Variants

Saccharomyces cerevisiae strain EBY100 (MATa URA3-52 trp1 leu2Δ1 his3Δ200 pep4::HIS3 prb1Δ1.6R can1 GAL (pIU211:URA3)) (Thermo Fisher Scientific) was used for yeast surface display.

Initially, cells were cultivated in YPD medium composed of 20 g/L peptone, 20 g/L dextrose and 10 g/L yeast extract supplemented with 10 ml/L penicillin-streptomycin (Gibco). Homologous recombination in yeast (gap repair cloning) was utilized for generation of a ΔB7-H6 (N-terminal Ig-like V-type domain (Asp25-Ala144) of the extracellular region of B7-H6) library. Eight residues of the N-terminal IgV-like domain of B7-H6 at the binding interface of B7-H6 and NKp30 (pdb: 4ZSO) were randomized via TRIM technology at GeneArt (Thermo Fisher Scientific). Sequences were cloned in a pYD-derived backbone as destination vector (pDest) in frame with Aga2p C-terminally carrying an HA epitope to enable yeast cell surface presentation and detection of full-length molecules (Roth et al., 2020). Cells harboring library plasmids were cultivated at 30° C. and 120 rpm in minimal SD-base medium with dropout mix composed of all essential amino acids except from tryptophan to maintain selection pressure according to the manufacturer’s instructions (Clontech), supplemented with 5.4 g/L Na₂HPO₄ and 8.6 g/L NaH₂PO₄ × H₂O. ΔB7-H6 expression for library sorting was achieved by culture of 10⁷ cells/ml for 48 h at 20° C. in SG medium with dropout mix wherein glucose was replaced by galactose (Clontech) and 10% (w/v) polyethylene glycol 800 (PEG 8000). NKp30 binding of yeast surface expressed ΔB7-H6 was monitored by indirect immunofluorescence using his-tagged NKp30 (Abcam) in combination with Penta-His Alexa Fluor 647 antibody (Qiagen, diluted 1:20). Simultaneously, ΔB7-H6 surface expression was detected by an anti-HA-PE antibody (Abcam, diluted 1:20). Detection and sorting of yeast candidates was done on a SH800S cell sorter (Sony) using a 70 µm sorting chip in three successive rounds. For the first sorting round 10⁸ yeast cells were incubated with 1 µM NKp30 followed by a second round with 100 nM NKp30 and a third round with 50 nM NKp30. Incubation was performed for 1 h on ice prior washing with PBS and sorting. For the second and third sorting round, cells were incubated for 30 min in 3 ml and 30 ml PBS, respectively, to increase sorting stringencies.

### Expression and Purification of NK Cell Engagers

Unique ΔB7-H6 sequences were fused to SEED AG chain and cloned into pTT5 (either in house or at GeneArt/Thermo Fisher Scientific) to allow full-length bispecific SEED production by combination with humanized Cetuximab (hu225) Fab on the SEED GA chain. Molecules were either produced in an effector silenced backbone by introduction of amino acid exchanges L234A, L235A, P329G (SEED_PGLALA) or in an effector competent IgG1 backbone (SEED). To this end, Expi293 cells were transiently transfected with respective expression vectors according to manufacturer’s instructions (Thermo Fisher Scientific). Antibody containing supernatants were harvested and purified via MabSelect antibody purification chromatography resin (GE Healthcare). NK cell engagers were dialyzed overnight against PBS pH 6.8 using Pur-A-Lyzer™ Maxi 3500 Dialysis Kit (Sigma Aldrich). Protein concentrations were determined by UV-Vis spectrophotometric measurement (Nanodrop ND-1000, Peqlab) and thermal stabilities were evaluated by differential scanning fluorimetry on a Prometheus NT.48 (Nanotemper Technologies). To assess purity and aggregations, proteins were analyzed by analytical size exclusion chromatography with a TSKgel SuperSW3000 column (4.6×300 mm, Tosoh Bioscience LLC) in an Agilent HPLC system with a flow rate of 0.35 ml/min.

### Biolayer Interferometry (BLI)

Kinetic measurements were performed on Octet RED96 system (ForteBio, Pall Life Science) at 25° C. and 1000 rpm. Immunoligands (5 µg/mL in PBS) were loaded on anti-human Fc biosensors for 5 min followed by 60 s rinsing the sensor with kinetics buffer (KB; PBS + 0.1 % Tween-20 + 1% BSA). Afterwards, association to human NKp30 (Abcam) in varying concentrations (15.6-1000 nM) was measured for 60 s followed by dissociation measurement for 180 s in KB. An irrelevant antigen was measured as negative control in each experiment. Data was fitted and analyzed with ForteBio data analysis software 8.0 using a 1:1 binding model after Savitzky-Golay filtering.

### Cell Culture

EGFR-expressing epidermoid carcinoma cell line A431 and non-small cell lung carcinoma cell line A549 were obtained from DSMZ (Deutsche Sammlung von Mikroorganismen und Zellkulturen, Braunschweig) and cultured in RPMI 1640 Glutamax-I or Dulbecco’s Modified Eagle’s medium supplemented with 10% FCS, 100 U/ml penicillin and 100 mg/ml streptomycin (R10+ and D10+), respectively (all components from Thermo Fisher Scientific). Human Expi293 cells for production of immunoligands were cultivated in suspension with complete Expi293 expression medium (Thermo Fisher Scientific).

### Tumor Cell Killings Assays

Preparation of PBMCs from healthy donors was performed as previously described (Repp et al., 2011). NK cells were isolated by negative selection using NK cell isolation kit (Miltenyi Biotech) and maintained overnight at a density of 2x10⁶ cells/ml in R10+ medium. Cytotoxicity was analyzed in standard 4 h ⁵¹Cr release assays performed in 96-well microtiter plates in a total volume of 200 µl as described previously (Repp et al., 2011). Human PBMCs or purified NK cells were used as effector cells at effector-to-target cell (E:T) ratios of 80:1 and 10:1, respectively. NK cell engagers or Cetuximab were applied at concentrations indicated. For blocking experiments, cells were pre-incubated for 15 min with 50 µg/ml of either anti-NKp30 mouse IgG2A antibody (mouse IgG2A isotype control antibody was used as a control in this experiments; both R&D Systems) to block NKp30 binding on NK cells or oa_hu225-SEED-PGLALA to block EGFR binding on tumor cells before the respective NK cell engagers were added. Percent lysis was calculated as follows from counts per minute (cpm): % lysis = (experimental cpm-basal cpm)/(maximal cpm-basal cpm)x100 and normalized (0 % = % lysis of oa _hu225-SEED-PGLALA control molecule, 100% = % lysis of Cetuximab at saturating concentration) to allow for direct comparison of affinity-matured ΔB7-H6-derived molecules between individual donors. Inhibited lysis was calculated as follows: 100% - (% lysis_{blocked}*100)/% lysis).

### Cytokine Release Assay

For cytokine release assays, NK cells were isolated with EasySep™ Human NK Cell Isolation Kit (Stemcell Technologies) and incubated overnight in AIM V medium containing 100 U/ml recombinant human interleukin-2 (R&D systems). 2,500 A431 cells were seeded in 384-well microtiter plates (Greiner Bio-One) and incubated for 3 h. Immunoligands were added to a final concentration of 85 nM followed by addition of NK cells at an E:T ratio of 5:1. After 24 h, supernatants were analyzed using human IFN-γ or TNF-α HTRF kit (Cisbio) according to manufacturer’s instructions. Plates were measured with PHERAstar FSX (BMG Labtech) and data were analyzed by MARS software (v.3.32, BMG) enabling a 4 parameter logistic (4PL 1/y²) model fitting of the standard curve.

### NK Cell Activation Assay

20,000 A431 cells/well were seeded in 96-well V-bottom microtiter plates (Thermo Fisher Scientific) and incubated for 3 h prior addition of 100,000 NK cells/well (E:T ratio of 5:1), which were treated with 100 U/ml recombinant human interleukin-2 overnight.. Immunoligands were added at a final concentration of 85 nM followed by 24 h incubation at 37° C. Cells were washed two times with PBS + 1% BSA prior incubation with LIVE/DEAD™ Fixable Near-IR Dead Cell Stain (Thermo Fisher Scientific), anti-human CD56-PE (Miltenyi Biotec) and anti-human CD69-APC (Abcam) for 1 h on ice. After washing, cells were measured via flow cytometry with Intellicyt® iQue® Screener Plus (Sartorius). For compensation of fluorochromes antibody capturing analysis beads (OneComp eBeads™ Compensation Beads, Thermo Fisher Scientific) were employed according to the manufacturer’s instructions.

### Data Processing and Statistical Analysis

Graphical and statistical analyses were performed with GraphPad Prism 8 software. P-values were calculated employing repeated measures ANOVA and the Bonferroni or Tukey post-test as recommended, or the student’s t-test when appropriate. p ≤0.05 were regarded as statistically significant.

All methods in Examples 2 to 7 were carried out as described in Example 1.

### Example 2

### Extracellular IgV-Like Domain of B7-H6 Is Sufficient to Engage NK Cells for Tumor Cell Lysis

In previous studies it was shown that B7-H6 scFv fusion proteins trigger NK cell-mediated lysis of tumor cells (Kellner et al., 2016). Since the NKp30 binding site is located in the IgV-like domain of B7-H6, it was tested if this isolated domain alone is sufficient to trigger NK cell activation and tumor cell killing.

To this end, the N-terminal Ig-like V-type domain (Asp25-Ala144) of the extracellular region of B7-H6 (termed ΔB7-H6; SEQ ID NO: 1) was used to design a novel EGFR-targeting NKp30 NK cell engager (FIG. 1A). To completely abolish Fc-mediated immune effector functions, the LALA-PG amino acid exchanges (L234A, L235A, P329G) were introduced (Schlothauer et al, 2016).

Kinetic studies of this ΔB7-H6_wt-SEED-PGLALA molecule (sequence shown in FIG. 1B) revealed functional binding to NKp30 (FIG. 1C) in accordance with previously published work (Li et al., 2011; Joyce et al., 2011). Most importantly, significant lysis of EGFR-expressing A431 tumor cells was triggered by this novel immunoligand (FIG. 1D), demonstrating that the isolated ΔB7-H6 domain is sufficient to promote NK cell-mediated lysis of target cells. However, the extent of tumor cell lysis with the monovalent EGFR binding NK cell engager AB7-H6_wt-SEED-PGLALA (EC₅₀ = 244.8 pM) was significantly reduced compared to a similarly designed one-armed (oa) EGFR-targeting molecule (oa _hu225-SEED) lacking the NKp30 binding domain but harboring an active IgG1 Fc region triggering FcyRIIIa, one of the strongest known trigger molecules expressed on NK cells (EC₅₀ = 27.8 pM).

### Example 3

### Affinity Maturation of the B7-H6IgV-Like Domain by Yeast Display

In order to improve the cytotoxic activity of the novel ΔB7-H6-based NK cell engager, a focused combinatorial mutant library of ΔB7-H6 for yeast surface display was designed using trinucleotide mutagenesis technology with the aim of enhancing affinity of ΔB7-H6 for NKp30. ΔB7-H6 was displayed successfully and showed specific binding to recombinant NKp30. Eight residues of B7-H6 (Ser60, Gly62, Phe82, Gly83, Val125, Thr127, Leu129 and Lys130) at the contact interface with NKp30 were chosen for library design. By randomization of these residues a library was comprising approximately 1×10⁷ unique clones synthesized and subsequently sorted by fluorescence-activated cell sorting (FACS) against recombinant NKp30 in reduced concentrations to isolate variants with significantly enhanced affinities. To this end, a two-dimensional labeling strategy was employed to simultaneously detect full-length ΔB7-H6 and binding to NKp30. Due to the low affinity interaction of B7-H6 and NKp30, that was reported to be in the µM range (Li et al., 2011), an NKp30 protein concentration of 1 µM was utilized in the first selection round and subsequently reduced to 100 nM and 50 nM, respectively.

Library output after three rounds of selection was analyzed for NKp30 binding in direct comparison with the wild-type ΔB7-H6 and revealed improved NKp30 binding of the vast majority of affinity-matured ΔB7-H6 clones. After sequencing of approximately 200 clones from sorting round two and three, multiple unique clones were identified (Table 1 and 2).

TABLE 1

Sequences of affinity-optimized ΔB7-H6 variants

| | |
|---|---|
| Identifier | Amino acid sequence |
| ΔB7-H6_S3#1 | DLKVEMMAGGTQITPLNDNVTIFCNIFYSQPLNITTMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#2 | DLKVEMMAGGTQITPLNDNVTIFCNIFYSQPLNITWMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#3 | DLKVEMMAGGTQITPLNDNVTIFCNIFYSQPLNITHMGITWFWKS LTFDKEVKVFEFFGDHQEAFRPGAIVSPWRLKSGDASLRLPGIQL EEAGEYRCEVFVTPYKAQGTVQLEVVASPAS |
| ΔB7-H6_S3#4 | DLKVEMMAGGTQITPLNDNVTIFCNIFYSQPLNITIMGITWFWKS LTFDKEVKVFEFWGDHQEAFRPGAIVSPWRLKSGDASLRLPGIQL EEAGEYRCEVVVTPLKAQGTVQLEVVASPAS |
| ΔB7-H6_S3#5 | DLKVEMMAGGTQITPLNDNVTIFCNIFYSQPLNITVMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#6 | DLKVEMMAGGTQITPLNDNVTIFCNIFYSQPLNITHMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#7 | DLKVEMMAGGTQITPLNDNVTIFCNIFYSQPLNITHMGITWFWKS LTFDKEVKVFEFYGDHQEAFRPGAIVSPWRLKSGDASLRLPGIQL EEAGEYRCEVVVYTPYKAQGTVQLEVVASPAS |
| ΔB7-H6_S3#8 | DLKVEMMAGGTQITPLNDNVTIFCNIFYSQPLNITHMGITWFWKS LTFDKEVKVFEFWGDHQEAFRPGAIVSPWRLKSGDASLRLPGIQL EEAGEYRCEVIVTPLKAQGTVQLEVVASPAS |
| ΔB7-H6_S3#9 | DLKVEMMAGGTQITPLNDNVTIFCNIFYSQPLNITHMGITWFWKS LTFDKEVKVFEFFGDHQEAFRPGAIVSPWRLKSGDASLRLPGIQL EEAGEYRCEVVVTPYKAQGTVQLEVVASPAS |
| ΔB7-H6_S3#10 | DLKVEMMAGGTQITPLNDNVTIFCNIFYSQPLNITSMGITWFWKS LTFDKEVKVFEFYGDHQEAFRPGAIVSPWRLKSGDASLRLPGIQL EEAGEYRCEVFVTPYKAQGTVQLEVVASPAS |
| ΔB7-H6_S3#11 | DLKVEMMAGGTQITPLNDNVTIFCNIFYSQPLNITLMGITWFWKS LTFDKEVKVFEFWGDHQEAFRPGAIVSPWRLKSGDASLRLPGIQL EEAGEYRCEVIVTPFKAQGTVQLEVVASPAS |
| ΔB7-H6_S3#12 | DLKVEMMAGGTQITPLNDNVTIFCNIFYSQPLNITEMGITWFWKS LTFDKEVKVFEFFGDHQEAFRPGAIVSPWRLKSGDASLRLPGIQL EEAGEYRCEVVVTPYKAQGTVQLEVVASPAS |
| ΔB7-H6_S3#13 | DLKVEMMAGGTQITPLNDNVTIFCNIFYSQPLNITSMIITWFWKS LTFDKEVKVFEFYGDHQEAFRPGAIVSPWRLKSGDASLRLPGIQL EEAGEYRCEVVVTPLKAQGTVQLEVVASPAS |
| ΔB7-H6_S3#14 | DLKVEMMAGGTQITPLNDNVTIFCNIFYSQPLNITTMGITWFWKS LTFDKEVKVFEFYGDHQEAFRPGAIVSPWRLKSGDASLRLPGIQL EEAGEYRCEVVVTPYKAQGTVQLEVVASPAS |
| ΔB7-H6_S3#15 | DLKVEMMAGGTQITPLNDNVTIFCNIFYSQPLNITIMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#16 | DLKVEMMAGGTQITPLNDNVTIFCNIFYSQPLNITLMGITWFWKS LTFDKEVKVFEFYGDHQEAFRPGAIVSPWRLKSGDASLRLPGIQL EEAGEYRCEVVVTPYKAQGTVQLEVVASPAS |
| ΔB7-H6_S3#17 | DLKVEMMAGGTQITPLNDNVTIFCNIFYSQPLNITSMGITWFWKS LTFDKEVKVFEFYGDHQEAFRPGAIVSPWRLKSGDASLRLPGIQL EEAGEYRCEVFVTPLKAQGTVQLEVVASPAS |
| ΔB7-H6_S3#18 | DLKVEMMAGGTQITPLNDNVTIFCNIFYSQPLNITYMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#19 | DLKVEMMAGGTQITPLNDNVTIFCNIFYSQPLNITQMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#20 | DLKVEMMAGGTQITPLNDNVTIFCNIFYSQPLNITHMGITWFWKS LTFDKEVKVFEFWGDHQEAFRPGAIVSPWRLKSGDASLRLPGIQL EEAGEYRCEVFVTPLKAQGTVQLEVVASPAS |
| ΔB7-H6_S3#21 | DLKVEMMAGGTQITPLNDNVTIFCNIFYSQPLNITLMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#22 | DLKVEMMAGGTQITPLNDNVTIFCNIFYSQPLNITEMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S3#23 | DLKVEMMAGGTQITPLNDNVTIFCNIFYSQPLNITEMGITWFWKS LTFDKEVKVFEFYGDHQEAFRPGAIVSPWRLKSGDASLRLPGIQL EEAGEYRCEVVVTPYKAQGTVQLEVVASPAS |
| ΔB7-H6_S3#24 | DLKVEMMAGGTQITPLNDNVTIFCNIFYSQPLNITIMGITWFWKS LTFDKEVKVFEFYGDHQEAFRPGAIVSPWRLKSGDASLRLPGIQL EEAGEYRCEVVVTPYKAQGTVQLEVVASPAS |
| ΔB7-H6_S3#25 | DLKVEMMAGGTQITPLNDNVTIFCNIFYSQPLNITWMGITWFWKS LTFDKEVKVFEFYGDHQEAFRPGAIVSPWRLKSGDASLRLPGIQL EEAGEYRCEVVVTPYKAQGTVQLEVVASPAS |
| ΔB7-H6_S3#26 | DLKVEMMAGGTQITPLNDNVTIFCNIFYSQPLNITTMGITWFWKS LTFDKEVKVFEFWDDHQEAFRPGAIVSPWRLKSGDASLRLPGIQL EEAGEYRCEVVVTPYKAQGTVQLEVVASPAS |
| ΔB7-H6_S3#27 | DLKVEMMAGGTQITPLNDNVTIFCNIFYSQPLNITTMGITWFWKS LTFDKEVKVFEFWGDHQEAFRPGAIVSPWRLKSGDASLRLPGIQL EEAGEYRCEVIVTPYKAQGTVQLEVVASPAS |
| ΔB7-H6_S3#28 | DLKVEMMAGGTQITPLNDNVTIFCNIFYSQPLNITHMGITWFWKS LTFDKEVKVFEFWGDHQEAFRPGAIVSPWRLKSGDASLRLPGIQL EEAGEYRCEVVVTPLKAQGTVQLEVVASPAS |
| ΔB7-H6_S3#29 | DLKVEMMAGGTQITPLNDNVTIFCNIFYSQPLNITVMGITWFWKS LTFDKEVKVFEFWGDHQEAFRPGAIVSPWRLKSGDASLRLPGIQL EEAGEYRCEVIVTPYKAQGTVQLEVVASPAS |
| ΔB7-H6_S3#30 | DLKVEMMAGGTQITPLNDNVTIFCNIFYSQPLNITEMGITWFWKS LTFDKEVKVFEFWGDHQEAFRPGAIVSPWRLKSGDASLRLPGIQL EEAGEYRCEVIVTPLKAQGTVQLEVVASPAS |
| ΔB7-H6_S3#31 | DLKVEMMAGGTQITPLNDNVTIFCNIFYSQPLNITIMGITWFWKS LTFDKEVKVFEFYGDHQEAFRPGAIVSPWRLKSGDASLRLPGIQL EEAGEYRCEVVVTPLKAQGTVQLEVVASPAS |
| ΔB7-H6_S3#32 | DLKVEMMAGGTQITPLNDNVTIFCNIFYSQPLNITEMGITWFWKS LTFDKEVKVFEFWGDHQEAFRPGAIVSPWRLKSGDASLRLPGIQL EEAGEYRCEVVVTPVKAQGTVQLEVVASPAS |
| ΔB7-H6_S2#1 | DLKVEMMAGGTQITPLNDNVTIFCNIFYSQPLNITSMGITWFWKS LTFDKEVKVFEFYGDHQEAFRPGAIVSPWRLKSGDASLRLPGIQL EEAGEYRCEVVVTPYKAQGTVQLEVVASPAS |
| ΔB7-H6_S2#2 | DLKVEMMAGGTQITPLNDNVTIFCNIFYSQPLNITSMGITWFWKS LTFDKEVKVFEFWDDHQEAFRPGAIVSPWRLKSGDASLRLPGIQL EEAGEYRCEVVVTPYKAQGTVQLEVVASPAS |
| ΔB7-H6_S2#3 | DLKVEMMAGGTQITPLNDNVTIFCNIFYSQPLNITTMGITWFWKS LTFDKEVKVFEFWGDHQEAFRPGAIVSPWRLKSGDASLRLPGIQL EEAGEYRCEVVVTPLKAQGTVQLEVVASPAS |
| ΔB7-H6_S2#4 | DLKVEMMAGGTQITPLNDNVTIFCNIFYSQPLNITHMGITWFWKS LTFDKEVKVFEFYADHQEAIRPGAIVSPWRLKSGDASLRLPGIQL EEAGEYRCEVVVTPYKAQGTVQLEVVASPAS |
| ΔB7-H6_S2#5 | DLKVEMMAGGTQITPLNDNVTIFCNIFYSQPLNITSMGITWLWKS LTFDKEVKVFEFWGDHQEAFRPGAIVSPWRLKSGDASLRLPGIQL EEAGEYRCEVFVTPYKAQGTVQLEVVASPAS |
| ΔB7-H6_S2#6 | DLKVEMMAGGTQITPLNDNVTIFCNIFYFQPLNITEMGITWFWKS LTFDKEVKVFEFFGDHQEAFRPGAIVSPWRLKSGDASLRLPGIQL EEAGEYRCEVIVTPLKAQGTVQLEVVASPAS |
| ΔB7-H6_S2#7 | DLKVEMMAGGTQITPLNDNVTIFCNIFYSQPLNITSMGITWFWKS LTFDKEVKVFEFYGDHQEAFRPGAIVSPWRLKSGDASLRLPGIQL EEAGEYRCEVIVTPYKAQGTVQLEVVTSPAS |
| ΔB7-H6_S2#8 | DLKVEMMAGGTQITPLNDNVTIFCNIFYSQPLNITTMGITWFWKS LTFDKEVKVFEFWGDHQEAFRPGAIVSPWRLKSGDASLRLPGIQL EEAGEYRCGVVVTPVKAQGTVQLEVVASPAS |
| ΔB7-H6_S2#9 | DLKVEMMAGGTQITPLNDNVTIFCNIFYSQPLNITSMGHITWFWKS LTFDKEVKVFEFYGDHQEAFRPGAIVSPWRLKSGDASLRLPGIQL EEAGEYRCEVVVTPLKAQGTAQLEVVASPAS |
| ΔB7-H6_S2#10 | DLKVEMMAGGTQITPLNDNVTIFCNIFYSQPLNITLMGITWFWKS LTFDKEVKVFEFWGDHQEAFRPGAIVSPWGLKSGDASLRLPGIQL EEAGEYRCEVVVTPLKAQGTVQLEVVASPAS |
| ΔB7-H6_S2#11 | DLKVEMMAGGTQITPLNDNVTIFCNIFYSQPLNITTMGITWFWKS LTFDKEVKVFEFWQDHQEAFRPGAIVSPWRLKSGDASLRLPGIQL EEAGEYRCEVVVTPLKAQGTVQLEVVASPAS |
| ΔB7-H6_S2#12 | DLKVEMMAGGTQITPLNDNVTIFCNIFYSQPLNITHMGITWLWKS LTFDKEVKVFEFYGDHQEAFRPGAIVSPWRLKSGDASLRLPGIQL EEAGEYRCEVVVTPLKAQGTVQLEVVASPAS |
| ΔB7-H6_S2#13 | DLKVEMMAGGTQITPLNDNVTIFCNIFYSQPLNITSMGITWFWKS LTFDKEVKVFEFWGDHQEAFRPGAIVSPWRLKSGDASLRLPGIQL EEAGEYRCEVVVTPYKAQGTVQLEVVASPAS |
| ΔB7-H6_S2#14 | DLKVEMMAGGTQITPLNDNVTIFCNIFYSQPLNITTMGITWFWKS LTFDKEVKVFEFFGDHQEAFRPGAIVSPWRLKSGDASLRLPGIQL EEAGEYRCEVVVTPYKAQGTVQLEVVASPAS |
| ΔB7-H6_S2#15 | DLKVEMMAGGTQITPLNDNVTIFCNIFYSQPLNITEMGITWFWKS LTFDKEVKVFEFYGDHQEAFXPGAIVSPWRLKSGDASLXLPGIQL EEAGEYRCEVVVTPLKAQGTVQLEVVASPAS |

TABLE 2

Mutations and maintained residues in the affinity-optimized ΔB7-H6 variants compared to wild-type B7-H6 (positions corresponding to Ser60, Gly62, Phe82, Gly83, Val125, Thr127, Leu129 and Lys130 of full-length wild-type B7-H6)

| | | | | | | | | |
|---|---|---|---|---|---|---|---|---|
| | Ser60 | Gly62 | Phe82 | Gly83 | Val125 | Thr127 | Leu129 | Lys130 |
| ΔB7-H6_wt | S | G | F | G | V | T | L | K |
| ΔB7-H6_S3#1 | T | G | W | G | V | T | Y | K |
| ΔB7-H6_S3#2 | W | G | W | G | V | T | Y | K |
| ΔB7-H6_S3#3 | H | G | F | G | F | T | Y | K |
| ΔB7-H6_S3#4 | I | G | W | G | V | T | L | K |
| ΔB7-H6_S3#5 | V | G | W | G | V | T | Y | K |
| ΔB7-H6_S3#6 | H | G | W | G | V | T | Y | K |
| ΔB7-H6_S3#7 | H | G | Y | G | V | T | Y | K |
| ΔB7-H6_S3#8 | H | G | W | G | I | T | L | K |
| ΔB7-H6_S3#9 | H | G | F | G | V | T | Y | K |
| ΔB7-H6_S3#10 | S | G | Y | G | F | T | Y | K |
| ΔB7-H6_S3#11 | L | G | W | G | I | T | F | K |
| ΔB7-H6_S3#12 | E | G | F | G | V | T | Y | K |
| ΔB7-H6_S3#13 | S | I | Y | G | V | T | L | K |
| ΔB7-H6_S3#14 | T | G | Y | G | V | T | Y | K |
| ΔB7-H6_S3#15 | I | G | W | G | V | T | Y | K |
| ΔB7-H6_S3#16 | L | G | Y | G | V | T | Y | K |
| ΔB7-H6_S3#17 | S | G | Y | G | F | T | L | K |
| ΔB7-H6_S3#18 | Y | G | W | G | V | T | Y | K |
| ΔB7-H6_S3#19 | Q | G | W | G | V | T | Y | K |
| ΔB7-H6_S3#20 | H | G | W | G | F | T | L | K |
| ΔB7-H6_S3#21 | L | G | W | G | V | T | Y | K |
| ΔB7-H6_S3#22 | E | G | W | G | V | T | Y | K |
| ΔB7-H6_S3#23 | E | G | Y | G | V | T | Y | K |
| ΔB7-H6_S3#24 | I | G | Y | G | V | T | Y | K |
| ΔB7-H6_S3#25 | W | G | Y | G | V | T | Y | K |
| ΔB7-H6_S3#26 | T | G | W | D | V | T | Y | K |
| ΔB7-H6_S3#27 | T | G | W | G | I | T | Y | K |
| ΔB7-H6_S3#28 | H | G | W | G | V | T | L | K |
| ΔB7-H6_S3#29 | V | G | W | G | I | T | Y | K |
| ΔB7-H6_S3#30 | E | G | W | G | I | T | L | K |
| ΔB7-H6_S3#31 | I | G | Y | G | V | T | L | K |
| ΔB7-H6_S3#32 | E | G | W | G | V | T | V | K |
| -ΔB7-H6_S2#1 | S | G | Y | G | V | T | Y | K |
| ΔB7-H6_S2#2 | S | G | W | D | V | T | Y | K |
| ΔB7-H6_S2#3 | T | G | W | G | V | T | L | K |
| ΔB7-H6_S2#4 | H | G | Y | A | V | T | Y | K |
| ΔB7-H6_S2#5 | S | G | W | G | F | T | Y | K |
| ΔB7-H6_S2#6 | E | G | F | G | I | T | L | K |
| ΔB7-H6_S2#7 | S | G | Y | G | I | T | Y | K |
| ΔB7-H6_S2#8 | T | G | W | G | V | T | V | K |
| ΔB7-H6_S2#9 | S | G | Y | G | V | T | L | K |
| ΔB7-H6_S2#10 | L | G | W | G | V | T | L | K |
| ΔB7-H6_S2#11 | T | G | W | Q | V | T | L | K |
| ΔB7-H6_S2#12 | H | G | Y | G | V | T | L | K |
| ΔB7-H6_S2#13 | S | G | W | G | V | T | Y | K |
| ΔB7-H6_S2#14 | T | G | F | G | V | T | Y | K |
| ΔB7-H6_S2#15 | E | G | Y | G | V | T | L | K |

Interestingly, none of those comprised mutations at positions Thr127 or Lys130. This may indicate that these two residues are essential for B7-H6 binding to NKp30 or for structural integrity of the molecule.

### Example 4

### Generation and Characterization of Affinity-Matured ΔB7-H6 NK Cell Engagers

47 unique affinity-matured ΔB7-H6 clones were reformatted as Fc-silenced SEEDbodies with monovalent EGFR binding by using the humanized Fab arm of Cetuximab (hu225) in the same way as described above for the wild-type ΔB7-H6 NK cell engager (FIG. 1A).

As summarized in Table 3, besides two variants that showed either very low expression (S2#8) or no productivity at all (S2#9), expression yields of all variants after protein A chromatography were in the double to triple digit milligram per liter scale. This is generally acceptable for transient expression of bifunctional antibody-like fusion proteins. Moreover, melting temperatures of all variants were quite similar to the denaturation point of the SEEDbody comprising ΔB7-H6 wild-type domain, giving clear evidence that no substantial loss in stability was observed by introducing mutations into ΔB7-H6.

TABLE 3

Biochemical and biophysical characterization of immunoligands based on affinity-optimized ΔB7-H6 variants

| Identifier | KD (M) | Kon (1/Ms) | Koff (1/s) | Tm (°C) | SEC (%) | Expression yield (mg/L) |
|---|---|---|---|---|---|---|
| ΔB7-H6_wt | 4.09E-07 | 4.12E+05 | 1.68E-01 | 64 | 92 | 136 |
| ΔB7-H6_S3#1 | 3.91E-08 | 7.54E+05 | 2.95E-02 | 63 | 95 | 111 |
| ΔB7-H6_S3#2* | 1.17E-08 | 1.04E+06 | 1.22E-02 | 63 | 75 | 57 |
| ΔB7-H6_S3#3 | 1.04E-07 | 1.05E+06 | 1.09E-01 | 63 | 93 | 82 |
| ΔB7-H6_S3#4* | 2.39E-08 | 6.21E+05 | 1.49E-02 | 63 | 81 | 23 |
| ΔB7-H6_S3#5 | 2.25E-08 | 7.99E+05 | 1.80E-02 | 63 | 89 | 27 |
| ΔB7-H6_S3#6 | 3.31E-08 | 7.37E+05 | 2.44E-02 | 63 | 96 | 39 |
| ΔB7-H6_S3#7 | 1.10E-07 | 4.78E+05 | 5.25E-02 | 63 | 96 | 43 |
| ΔB7-H6_S3#8 | 8.07E-08 | 5.64E+05 | 4.55E-02 | 63 | 86 | 27 |
| ΔB7-H6_S3#9 | 5.78E-08 | 9.70E+05 | 5.60E-02 | 63 | 96 | 41 |
| ΔB7-H6_S3#10 | 1.45E-07 | 6.89E+05 | 9.98E-02 | 63 | 91 | 34 |
| ΔB7-H6_S3#11 | 3.91E-08 | 1.11E+06 | 4.32E-02 | 63 | 89 | 19 |
| ΔB7-H6_S3#12 | 1.29E-07 | 6.71E+05 | 8.65E-02 | 63 | 95 | 51 |
| ΔB7-H6_S3#13* | Non binding | - | - | 64 | 94 | 23 |
| ΔB7-H6_S3#14 | 3.45E-08 | 9.33E+05 | 3.22E-02 | 64 | 96 | 42 |
| ΔB7-H6_S3#15 | 1.24E-08 | 8.17E+05 | 1.01E-02 | 64 | 90 | 23 |
| ΔB7-H6_S3#16 | 2.87E-08 | 1.01E+06 | 2.89E-02 | 63 | 93 | 32 |
| ΔB7-H6_S3#17 | 5.91E-08 | 9.69E+05 | 5.73E-02 | 63 | 88 | 23 |
| ΔB7-H6_S3#18 | 9.06E-09 | 1.50E+06 | 1.36E-02 | 63 | 88 | 20 |
| ΔB7-H6_S3#19 | 1.46E-07 | 6.73E+05 | 9.81E-02 | 63 | 96 | 40 |
| ΔB7-H6_S3#20* | 6.16E-08 | 1.04E+06 | 6.42E-02 | 63 | 81 | 25 |
| ΔB7-H6_S3#21 | 1.79E-08 | 1.18E+06 | 2.11E-02 | 63 | 91 | 33 |
| ΔB7-H6_S3#22 | 9.52E-08 | 4.63E+05 | 4.41E-02 | 63 | 96 | 43 |
| ΔB7-H6_S3#23 | 1.10E-07 | 4.79E+05 | 5.25E-02 | 63 | 95 | 40 |
| ΔB7-H6_S3#24 | 1.49E-08 | 7.02E+05 | 1.04E-02 | 64 | 92 | 26 |
| ΔB7-H6_S3#25 | 6.52E-08 | 7.45E+05 | 4.86E-02 | 63 | 98 | 16 |
| ΔB7-H6_S3#26 | 1.13E-07 | 4.09E+05 | 4.64E-02 | 64 | 93 | 24 |
| ΔB7-H6_S3#27 | 8.30E-08 | 6.69E+05 | 5.56E-02 | 64 | 97 | 31 |
| ΔB7-H6_S3#28 | 4.43E-07 | 3.06E+05 | 1.36E-01 | 63 | 89 | 42 |
| ΔB7-H6_S3#29 | 2.48E-08 | 7.05E+05 | 1.75E-02 | 63 | 88 | 25 |
| ΔB7-H6_S3#30 | 1.25E-07 | 6.22E+05 | 7.76E-02 | 63 | 90 | 47 |
| ΔB7-H6_S3#31 | 4.36E-08 | 6.16E+05 | 2.69E-02 | 64 | 86 | 28 |
| ΔB7-H6_S3#32* | 1.88E-07 | 4.09E+05 | 7.66E-02 | 63 | 83 | 32 |
| ΔB7-H6_S2#1 | 2.05E-07 | 3.80E+05 | 7.80E-02 | 63 | 95 | 54 |
| ΔB7-H6_S2#2 | 2.17E-07 | 3.40E+05 | 7.37E-02 | 64 | 93 | 47 |
| ΔB7-H6_S2#3 | 1.94E-07 | 5.46E+05 | 1.06E-01 | 63 | 91 | 45 |
| ΔB7-H6_S2#4* | 1.47E-07 | 8.30E+05 | 1.22E-01 | 64 | 69 | 22 |
| ΔB7-H6_S2#5* | 2.53E-07 | 5.85E+05 | 1.48E-01 | 63 | 76 | 28 |
| ΔB7-H6_S2#6* | 2.85E-07 | 1.32E+06 | 3.76E-01 | 63 | 57 | 29 |
| ΔB7-H6_S2#7 | 1.91E-07 | 5.04E+05 | 9.64E-02 | 64 | 95 | 49 |
| ΔB7-H6_S2#8* | Non binding | - | - | 64 | 17 | 9 |
| ΔB7-H6_S2#9* | - | - | - | - | - | No expression yield obtained |
| ΔB7-H6_S2#10 | 1.87E-07 | 5.97E+05 | 1.11E-01 | 63 | 90 | 40 |
| ΔB7-H6_S2#11 | 2.34E-07 | 4.91E+05 | 1.15E-01 | 63 | 88 | 41 |
| ΔB7-H6_S2#12* | 2.91E-07 | 5.56E+05 | 1.62E-01 | 63 | 73 | 27 |
| ΔB7-H6_S2#13 | 2.05E-07 | 4.10E+05 | 8.42E-02 | 63 | 96 | 49 |
| ΔB7-H6_S2#14 | 2.17E-07 | 5.70E+05 | 1.24E-01 | 63 | 95 | 44 |
| ΔB7-H6_S2#15 | 3.50E-07 | 4.01E+05 | 1.40E-01 | 63 | 94 | 52 |

Expression yields were determined post protein A purification. SEC indicates target monomer peaks as determined by analytical size exclusion chromatography. Variants marked with an asterisk (*) were not applied for cellular assays due to their biophysical properties.

Most importantly, in comparison with the wild-type ΔB7-H6 NK cell engager with the only exception of ΔB7-H6_S3#28 all of the affinity-matured variants showed significantly enhanced affinities for NKp30 of up to 45.1-fold (S3#18) (Table 4).

TABLE 4

Biochemical characterization of immunoligands based on affinity-optimized ΔB7-H6 variants

| Identifier | KD (M) | x-fold KD improvement* | Kon (1/Ms) | Koff (1/s) | x-fold Koff improvement^{∗} | Mutations |
|---|---|---|---|---|---|---|
| ΔB7-H6_wt | 4.09E-07 | - | 4.12E+05 | 1.68E-01 | - | - |
| ΔB7-H6_S3#1 | 3.91E-08 | 10.5 | 7.54E+05 | 2.95E-02 | 5.7 | S60T, F82W, L129Y |
| ΔB7-H6_S3#3 | 1.04E-07 | 3.9 | 1.05E+06 | 1.09E-01 | 1.5 | S60H, V125F, L129Y |
| ΔB7-H6_S3#5 | 2.25E-08 | 18.2 | 7.99E+05 | 1.80E-02 | 9.3 | S60V, F82W, L129Y |
| ΔB7-H6_S3#6 | 3.31E-08 | 12.4 | 7.37E+05 | 2.44E-02 | 6.9 | S60H, F82W, L129Y |
| ΔB7-H6_S3#7 | 1.10E-07 | 3.7 | 4.78E+05 | 5.25E-02 | 3.2 | S60H, F82Y, L129Y |
| ΔB7-H6_S3#8 | 8.07E-08 | 5.1 | 5.64E+05 | 4.55E-02 | 3.7 | S60H, F82W, V125I |
| ΔB7-H6_S3#9 | 5.78E-08 | 7.1 | 9.70E+05 | 5.60E-02 | 3.0 | S60H, L129Y |
| ΔB7-H6_S3#10 | 1.45E-07 | 2.8 | 6.89E+05 | 9.98E-02 | 1.7 | F82Y, V125F, L129Y |
| ΔB7-H6_S3#11 | 3.91E-08 | 10.5 | 1.11E+06 | 4.32E-02 | 3.9 | S60L, F82W, V1251, L129F |
| ΔB7-H6_S3#12 | 1.29E-07 | 3.2 | 6.71E+05 | 8.65E-02 | 1.9 | S60E, L129Y |
| ΔB7-H6_S3#14 | 3.45E-08 | 11.9 | 9.33E+05 | 3.22E-02 | 5.2 | S60T, F82Y, L129Y |
| ΔB7-H6_S3#15 | 1.24E-08 | 33.0 | 8.17E+05 | 1.01E-02 | 16.6 | S60I, F82W, L129Y |
| ΔB7-H6_S3#16 | 2.87E-08 | 14.3 | 1.01E+06 | 2.89E-02 | 5.8 | S60L, F82Y, L129Y |
| ΔB7-H6_S3#17 | 5.91E-08 | 6.9 | 9.69E+05 | 5.73E-02 | 2.9 | F82Y, V125F |
| ΔB7-H6_S3#18 | 9.06E-09 | 45.1 | 1.50E+06 | 1.36E-02 | 12.4 | S60Y, F82W, L129Y |
| ΔB7-H6_S3#19 | 1.46E-07 | 2.8 | 6.73E+05 | 9.81E-02 | 1.7 | S60Q, F82W, L129Y |
| ΔB7-H6_S3#21 | 1.79E-08 | 22.8 | 1.18E+06 | 2.11E-02 | 8.0 | S60L, F82W, L129Y |
| ΔB7-H6_S3#22 | 9.52E-08 | 4.3 | 4.63E+05 | 4.41E-02 | 3.8 | S60E, F82W, L129Y |
| ΔB7-H6_S3#23 | 1.10E-07 | 3.7 | 4.79E+05 | 5.25E-02 | 3.2 | S60E, F82Y, L129Y |
| ΔB7-H6_S3#24 | 1.49E-08 | 27.5 | 7.02E+05 | 1.04E-02 | 16.1 | S60I, F82Y, L129Y |
| ΔB7-H6_S3#25 | 6.52E-08 | 6.3 | 7.45E+05 | 4.86E-02 | 3.5 | S60W, F82Y, L129Y |
| ΔB7-H6_S3#26 | 1.13E-07 | 3.6 | 4.09E+05 | 4.64E-02 | 3.6 | S60T, F82W, G83D, L129Y |
| ΔB7-H6_S3#27 | 8.30E-08 | 4.9 | 6.69E+05 | 5.56E-02 | 3.0 | S60T, F82W, V1251, L129Y |
| ΔB7-H6_S3#28 | 4.43E-07 | 0.9 | 3.06E+05 | 1.36E-01 | 1.2 | S60H, F82W |
| ΔB7-H6_S3#29 | 2.48E-08 | 16.5 | 7.05E+05 | 1.75E-02 | 9.6 | S60V, F82W, V125I. L129Y |
| ΔB7-H6_S3#30 | 1.25E-07 | 3.3 | 6.22E+05 | 7.76E-02 | 2.2 | S60E, F82W, V125I |
| ΔB7-H6 _S3#31 | 4.36E-08 | 9.4 | 6.16E+05 | 2.69E-02 | 6.3 | S60I, F82Y |
| ΔB7-H6_S2#1 | 2.05E-07 | 2.0 | 3.80E+05 | 7.80E-02 | 2.2 | F82Y, L129Y |
| ΔB7-H6_S2#2 | 2.17E-07 | 1.9 | 3.40E+05 | 7.37E-02 | 2.3 | F82W, L129Y |
| ΔB7-H6_S2#3 | 1.94E-07 | 2.1 | 5.46E+05 | 1.06E-01 | 1.6 | S60T, F82W |
| ΔB7-H6_S2#7 | 1.91E-07 | 2.1 | 5.04E+05 | 9.64E-02 | 1.7 | F82Y, V1251, L129Y |
| ΔB7-H6_S2#10 | 1.87E-07 | 2.2 | 5.97E+05 | 1.11E-01 | 1.5 | S60L, F82W |
| ΔB7-H6_S2#11 | 2.34E-07 | 1.7 | 4.91E+05 | 1.15E-01 | 1.5 | S60T, F82W |
| ΔB7-H6_S2#13 | 2.05E-07 | 2.0 | 4.10E+05 | 8.42E-02 | 2.0 | F82W, L129Y |
| ΔB7-H6_S2#14 | 2.17E-07 | 1.9 | 5.70E+05 | 1.24E-01 | 1.4 | S60T, L129Y |
| ΔB7-H6_S2#15 | 3.50E-07 | 1.2 | 4.01E+05 | 1.40E-01 | 1.2 | S60E, F82Y |

X-fold improvement values represent the improvement factor of respective overall affinities and off-rates against NKp30 compared to the ΔB7-H6 wild-type NK cell engager. Expression yields were determined post protein A purification. SEC indicates target monomer peaks as determined by analytical size exclusion chromatography. Mutations describes the incorporated amino acid exchanges of the variants compared to the ΔB7-H6 wild-type NK cell engager. All molecules were expressed as Fc immune effector silenced variants harboring the LALA-PG amino acid exchanges.

In general, improved off-rates contributed substantially more to the overall affinity maturation than modulations of on-rates of the ΔB7-H6 SEED PGLALA immunoligands.

Interestingly, variant S3#13 that did not bind NKp30, was the only clone isolated that contained a mutation of Gly62 (Gly62Ile). This observation suggests that in addition to Thr127 or Lys130, Gly62 may also be indispensable for ΔB7-H6 binding to NKp30.

The different affinity-maturated ΔB7-H6 immunoligands were grouped based on the affinity of their respective ΔB7-H6 variant for NKp30. Frequencies of these mutations in the different groups are depicted graphically in FIG. 2.

Three crucial amino acid residues for the binding to NKp30 can be de deduced from the mutation frequency. Glycine at position 62, threonine at position 127 and lysine at position 130 are necessary to provide receptor binding of B7-H6 to NKp30. This conclusion is also supported by the fact that the replacement of glycine at position 62 by a isoleucine in variant ΔB7-H6_S3#13 led to a total loss of NKp30 binding.

For subsequent functional evaluation of the affinity-matured ΔB7-H6-derived NK cell engagers, immunoligands were selected that showed at least 85% monomers (i.e. a maximum of 15% aggregates) as determined by size exclusion chromatography. Accordingly, 36 affinity-matured ΔB7-H6 NK cell engagers were selected for functional experiments (Table 4).

### Example 5

### Affinity-Matured ΔB7-H6 Immunoligands Elicit Strongly Enhanced NK Cell-Mediated Cytotoxicity Against EGFR-Expressing Tumor Cells

As an initial screening, the 36 remaining ΔB7-H6, EGFR-targeting NK cell engagers were tested for their capacity to mediate tumor cell killing using PBMCs (containing 5-20% NK cells) from healthy donors as effector cells and the EGFR-positive tumor cell line A431 as target cells in chromium release assays. To allow comparison and ranking of the 36 molecules, data sets derived from different effector cell donors, who usually show varying donor-dependent maximal lytic activity, were normalized. An internal positive control was set to 100% and oa _hu225-SEED-PGLALA-mediated lysis to 0% since this molecule is functionally inactive and reflects the tumor cell lysis induced by the NK cells alone (FIG. 1D).

In these experiments, the Fc effector silenced wild-type ΔB7-H6 NK cell engager ΔB7H6_wt SEED-PGLALA triggered significant tumor cell lysis (EC₅₀ = 1.1 nM). Of note, all 36 ΔB7-H6 affinity-matured SEED-PGLALA molecules mediated higher maximum tumor cell lysis and achieved enhanced potency of up to 187-fold (S3#25) compared to the wild-type ΔB7-H6 immunoligand. Among them, 11 constructs bound to NKp30 with affinity improvements of more than 10-fold (KD < 40 nM) and five variants comprised enhanced affinities between 5-fold and 10-fold, whereas the majority of clones comprised optimized affinities of less than 5-fold (KD > 120 nM; Table 4). Six of the 11 NK cell engagers constructed with the high-affinity ΔB7-H6 clones (KD < 40 nM), i.e. S3#14, S3#15, S3#16, S3#18, S3#24 and S3#29, induced NK cell-mediated killing of tumor cells with EC₅₀ values ranging from 10.6-71.1 pM, representing a 16-fold to 106-fold increase compared to the wild-type ΔB7-H6 NK cell engager. Of the NK cell engagers using ΔB7-H6 clones with optimized affinities for NKp30 between 5-fold and 10-fold, S3#25 and S3#31 were the most potent, displaying NKp30 affinity enhancements of 6.3-fold and 9.4-fold, respectively. These molecules induced NK cell-mediated killing of tumor cells with half-maximal killing activity of 6 pM (S3#25) and 25.1 pM (S3#31). From the variants comprising optimized NKp30 affinities of less than 5-fold, clone S3#27 (KD = 83 nM) elicited significant tumor cell lysis (EC₅₀ = 27.6 pM) with 41-fold improved potency compared to wild-type ΔB7-H6 and was also selected for further characterization.

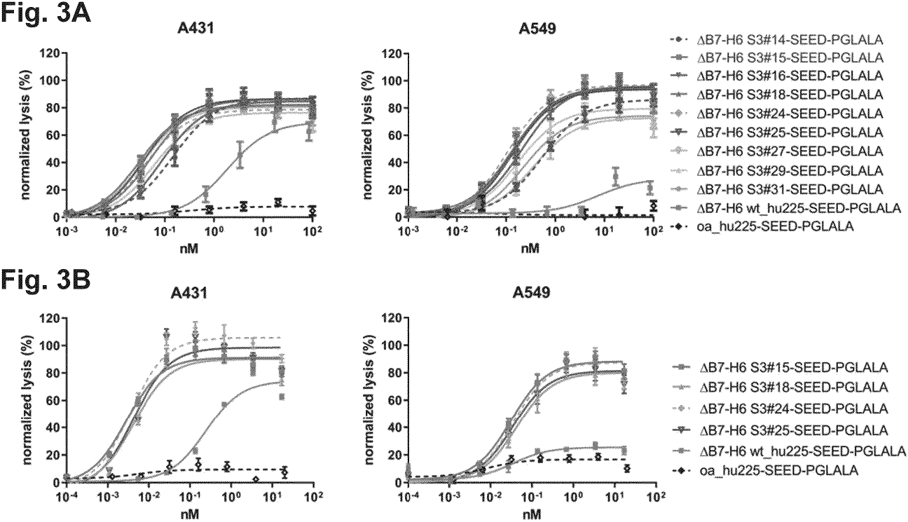

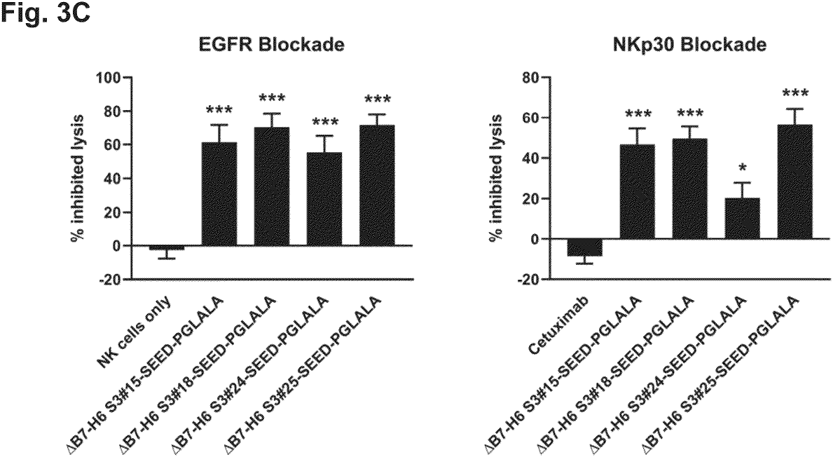

Next, the potency of the nine most active candidates from the initial screening (i.e. S3#14, S3#15, S3#16, S3#18, S3#24, S3#25, S3#27, S3#29, S3#31) was further compared via chromium release assays using A431 and A549 cells with PBMCs from 3 different donors (FIG. 3A). As observed for A431 cells, the nine candidates also mediated substantially improved maximum killing and significantly enhanced potency in tumor cell lysis compared to the NK cell engager harboring wild-type ΔB7-H6 against tumor cell line A549 expressing significantly lower EGFR levels compared to A431 cells (FIG. 3A). While wild-type ΔB7-H6 NK cell engager ΔB7H6_wt SEED-PGLALA showed significant, but limited activity (EC₅₀_{A431} = 1.8 nM), the most active affinity-optimized NK cell engager S3#15 demonstrated improved potency at low pM concentrations with PBMCs of 3 different donors (EC₅₀ = 29.9 pM). Extent of lysis of A549 cells achieved by the most effective affinity-matured ΔB7-H6 NK cell engager S3#24 (EC₅₀ = 108.3 pM), is about 3.6-times higher compared to A431 cells (Table 5). This might most likely reflect the impact of the lower EGFR expression level on A549 target cells, a well-known parameter affecting effector cell killing.

TABLE 5

Potencies of selected EGFR-targeting, affinity-matured ΔB7-H6 immunoligands.

| Molecule | A431 EC₅₀ with PBMC (pM) | A549 EC₅₀ with PBMC (pM) | A431 EC₅₀ with NK cells (pM) | A549 EC₅₀ with NK cells (pM) |
|---|---|---|---|---|
| oa_hu225 | no lysis | no lysis | no lysis | no lysis |
| ΔB7-H6_wt | 1839 | n.a. | 244.8 | n.a. |
| ΔB7-H6_S3#14 | 150.1 | 556.5 | n.d. | n.d. |
| ΔB7-H6_S3#15 | 29.9 | 133.2 | 2.80 | 31.31 |
| ΔB7-H6_S3#16 | 87.3 | 205.8 | n.d. | n.d. |
| ΔB7-H6_S3#18 | 51.1 | 132.0 | 4.31 | 42.47 |
| ΔB7-H6_S3#24 | 39.7 | 108.3 | 3.55 | 35.19 |
| ΔB7-H6_S3#25 | 37.8 | 134.9 | 4.25 | 34.62 |
| ΔB7-H6_S3#27 | 60.2 | 355.6 | n.d. | n.d. |
| ΔB7-H6_S3#29 | 33.6 | 172.3 | n.d. | n.d. |
| ΔB7-H6_S3#31 | 34.8 | 223.8 | n.d. | n.d. |

n.d. = not determined; n.a. = not applicable due to insignificant killing. All molecules were produced as Fc immune effector silenced variants harboring the LALA-PG amino acid exchanges.

To further characterize the potent NK cell engagers S3#15, S3#18, S3#24 and S3#25, killings assays with purified, non-stimulated NK cells were performed (FIG. 3B). Using A431 cells, the four ΔB7-H6 affinity-maturated NK cell engagers mediated tumor cell killing in the single digit picomolar range, representing an increase in potency of up to 87-fold (S3#15) compared to the NK cell engager harboring the wild-type ΔB7-H6 domain (Table 5). Even for A549 target cells, potencies of all four affinity-maturated NK cell engagers were still in the picomolar range. Notably, efficacy of wild-type ΔB7-H6 NK cell engager was not substantially different from the negative control in PBMC and NK cell ADCCs with A549 cells (FIGS. 3A, B).

Finally, EGFR-specific and NKp30-mediated killing by the four tested, affinity-maturated NK cell engagers was further verified by either blocking EGFR on cell surface of A431 cells or by pre-incubation of NK cells with an excess of an anti-human NKp30 antibody. As shown in FIG. 3C, lysis of A431 tumor cells was significantly inhibited for all four NK cell engagers under both conditions demonstrating the specificity and prerequisite of bispecific engagement of these novel B7-H6-based affinity-matured immunoligands (FIG. 3C).

Data obtained in the above-described chromium release experiment with A431 cells and with PBMCs from healthy donors is summarized in Table 6 below for a large number of different immunoligand constructs.

TABLE 6

Induced tumor cell death due to natural killer cell activation

| Molecule | EC50 (pM) | EC50 improvement compared to ΔB7-H6 wt | Max. killing (% lysis normalized to Cetuximab) | Max. killing improvement compared to ΔB7-H6_wt | KD (M) | KD improvement compared to ΔB7-H6_wt |
|---|---|---|---|---|---|---|
| ΔB7-H6_wt | 868 | 1 | 63 | 1.00 | 4.09E-07 | 1 |
| ΔB7-H6_S3#1 | 61.1 | 14 | 99 | 1.57 | 3.91E-08 | 10.5 |
| ΔB7-H6_S3#3 | 110 | 8 | 102 | 1.62 | 1.04E-07 | 3.9 |
| ΔB7-H6_S3#5 | 54.7 | 16 | 86 | 1.37 | 2.25E-08 | 18.2 |
| ΔB7-H6_S3#6 | 40.8 | 21 | 105 | 1.67 | 3.31E-08 | 12.4 |
| ΔB7-H6_S3#7 | 45.4 | 19 | 101 | 1.60 | 1.10E-07 | 3.7 |
| ΔB7-H6_S3#8 | 43.7 | 20 | 97 | 1.54 | 8.07E-08 | 5.1 |
| ΔB7-H6_S3#9 | 64.0 | 14 | 98 | 1.56 | 5.78E-08 | 7.1 |
| ΔB7-H6_S3#10 | 85.0 | 10 | 110 | 1.75 | 1.45E-07 | 2.8 |
| ΔB7-H6_S3#11 | 65.8 | 13 | 91 | 1.44 | 3.91E-08 | 10.5 |
| ΔB7-H6_S3#12 | 165 | 5 | 103 | 1.63 | 1.29E-07 | 3.2 |
| ΔB7-H6_S3#13 | No lysis | - | 8 | 0.13 | Non binding | - |
| ΔB7-H6_S3#14 | 21.0 | 41 | 89 | 1.41 | 3.45E-08 | 11.9 |
| ΔB7-H6_S3#15 | 8.7 | 100 | 89 | 1.41 | 1.24E-08 | 33.0 |
| ΔB7-H6_S3#16 | 29.1 | 30 | 114 | 1.81 | 2.87E-08 | 14.3 |
| ΔB7-H6_S3#17 | 427 | 2 | 75 | 1.19 | 5.91E-08 | 6.9 |
| ΔB7-H6_S3#18 | 19.8 | 44 | 100 | 1.59 | 9.06E-09 | 45.1 |
| ΔB7-H6_S3#19 | 240 | 4 | 82 | 1.30 | 1.46E-07 | 2.8 |
| ΔB7-H6_S3#21 | 36.1 | 24 | 86 | 1.37 | 1.79E-08 | 22.8 |
| ΔB7-H6_S3#22 | 106 | 8 | 88 | 1.40 | 9.52E-08 | 4.3 |
| ΔB7-H6_S3#23 | 78.0 | 11 | 94 | 1.49 | 1.10E-07 | 3.7 |
| ΔB7-H6_S3#24 | 9.3 | 93 | 85 | 1.35 | 1.49E-08 | 27.4 |
| ΔB7-H6_S3#25 | 9.9 | 88 | 94 | 1.49 | 6.52E-08 | 6.3 |
| ΔB7-H6_S3#26 | 22.7 | 38 | 88 | 1.40 | 1.13E-07 | 3.6 |
| ΔB7-H6_S3#27 | 19.2 | 45 | 113 | 1.79 | 8.30E-08 | 4.9 |
| ΔB7-H6_S3#28 | 114 | 8 | 100 | 1.59 | 4.43E-07 | 0.9 |
| ΔB7-H6_S3#29 | 7.6 | 114 | 80 | 1.27 | 2.48E-08 | 16.5 |
| ΔB7-H6_S3#30 | 176 | 5 | 88 | 1.40 | 1.25E-07 | 3.3 |
| ΔB7-H6_S3#31 | 17.5 | 50 | 94 | 1.49 | 4.36E-08 | 9.4 |
| ΔB7-H6_S2#1 | 140 | 6 | 104 | 1.65 | 2.05E-07 | 2.0 |
| ΔB7-H6_S2#2 | 169 | 5 | 76 | 1.21 | 2.17E-07 | 1.9 |
| ΔB7-H6_S2#3 | 175 | 5 | 86 | 1.37 | 1.94E-07 | 2.1 |
| ΔB7-H6_S2#7 | 170 | 5 | 96 | 1.52 | 1.91E-07 | 2.1 |
| ΔB7-H6_S2#10 | 122 | 7 | 99 | 1.57 | 1.87E-07 | 2.2 |
| ΔB7-H6_S2#11 | 172 | 5 | 93 | 1.48 | 2.34E-07 | 1.7 |
| ΔB7-H6_S2#13 | 171 | 5 | 107 | 1.70 | 2.05E-07 | 2.0 |
| ΔB7-H6_S2#14 | 158 | 5 | 99 | 1.57 | 2.17E-07 | 1.9 |
| ΔB7-H6_S2#15 | 544 | 2 | 90 | 1.43 | 3.50E-07 | 1.2 |

It could be observed that all tested ΔB7-H6 variants, except the non-binding ΔB7-H6_S3#13 molecule, activate NK cells in a specific manner (no lysis of EGFR-negative CHO cells).

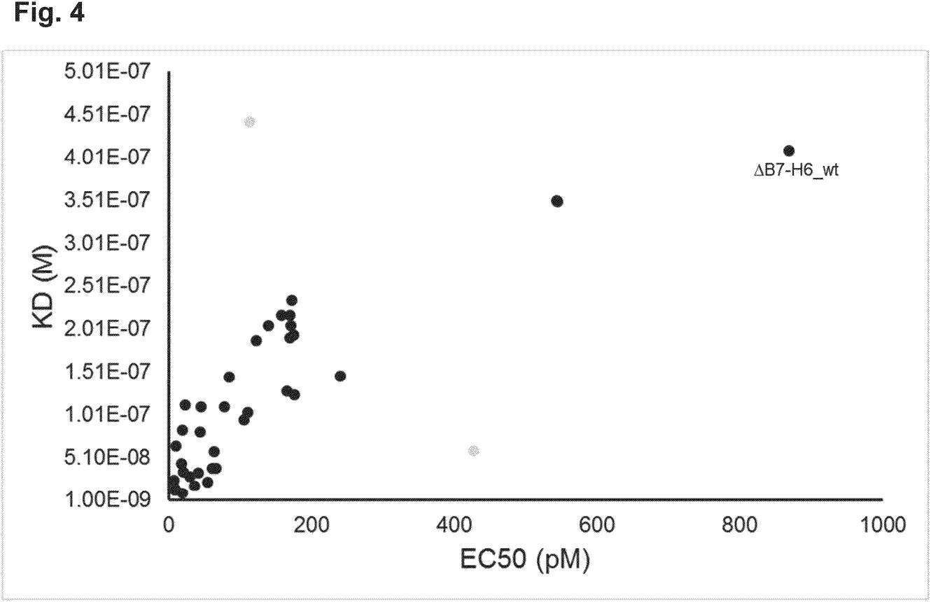

In a further analysis, the binding affinity for NKp30 of the different constructs was plotted against their EC50 in this killing assay (FIG. 4). This plot shows that the affinity of the ΔB7-H6 variants against NKp30 correlates with killing (EC50), clearly indicating that enhanced affinities translate into enhanced killing. Every data point depicts a single ΔB7-H6 variant. Outliers marked in light grey.

### Example 6

### ΔB7-H6 Affinity-Maturated NK Cell Engagers Exhibit a Distinct NK Cell Activation Profile

In this experiment, activation of NK cells in the presence of tumor cells overexpressing EGFR (A431) and four selected immunoligands was examined by analyzing CD69 upregulation as early NK cell activation marker.

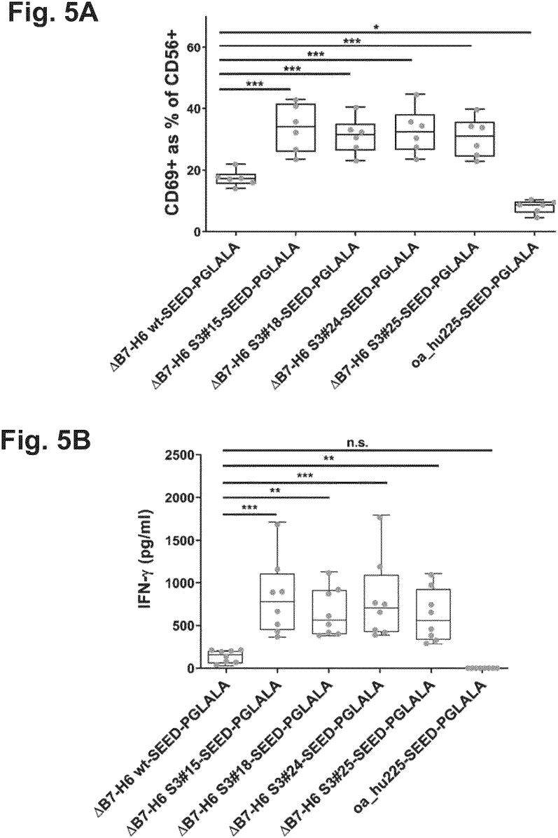

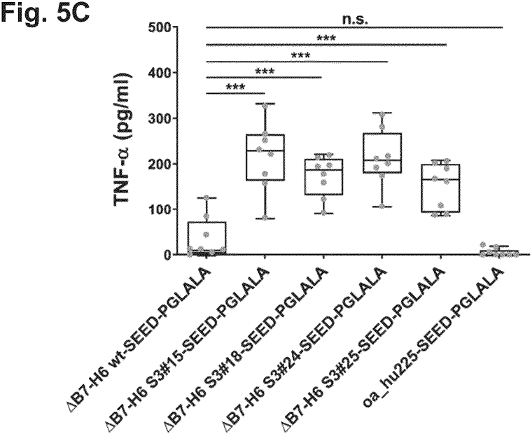

As expected, negative control (one-armed oa_hu225-SEED-PGLALA that lacks a ΔB7-H6 domain mediated neglectable NK cell activation, whereas the NK cell engager harboring wild-type ΔB7-H6 elicited CD69 expression on approximately 17% of NK cells on average (FIG. 5A). Compared to the wild-type ΔB7-H6 immunoligand, the tested affinity-optimized ΔB7-H6 immunoligands activated nearly twice as many NK cells ranging from 30.7% to 33.8% CD69-positive NK cells (FIG. 5A). In addition, all four tested NK cell engagers promoted significantly increased NK cell production of proinflammatory cytokines IFN-γ and TNF-α in a target-dependent manner (FIGS. 5B, C). In comparison to the wild-type ΔB7-H6 molecule, which induced release of 141 pg/ml IFN-γ and 37 pg/ml TNF-α, S3#15, S3#18, S3#24 and S3#25 induced release of 615-824 pg/ml IFN-γ and 152-214 pg/ml TNF-α on average (FIGS. 5B, C). Thus, affinity-engineered NK cell engagers induced up to 5.8-fold increased levels of IFN-γ and TNF-α compared to the wild-type ΔB7-H6 immunoligand. These findings are in line with the vastly enhanced killing potencies and efficacies of the affinity-engineered ΔB7-H6 NK cell engagers observed before.

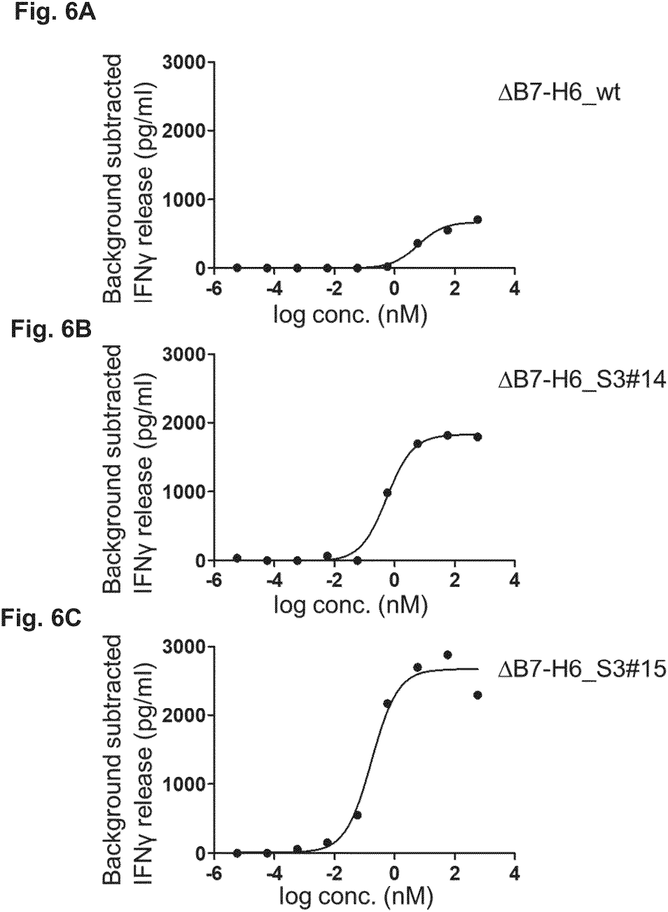

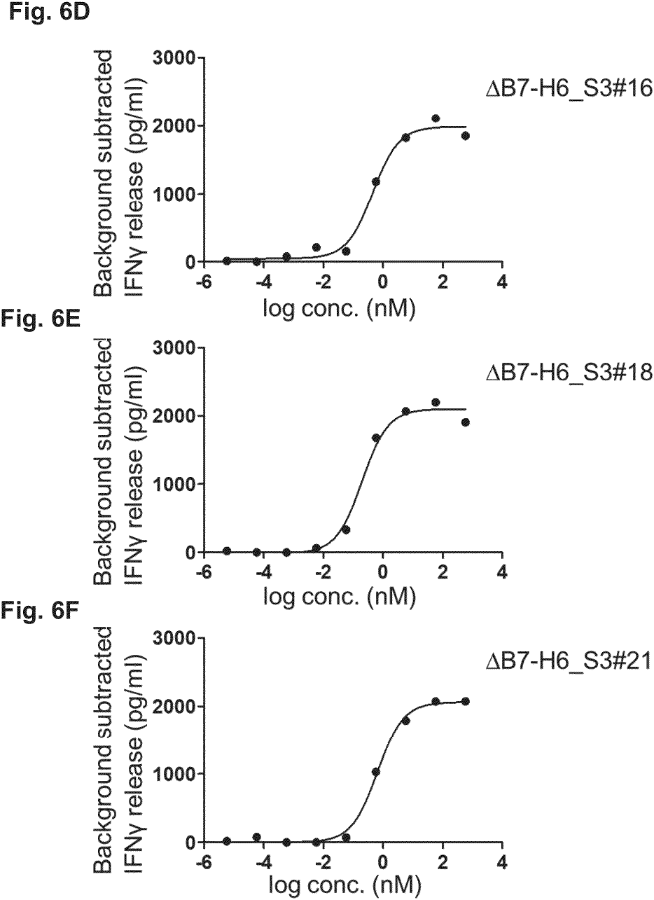

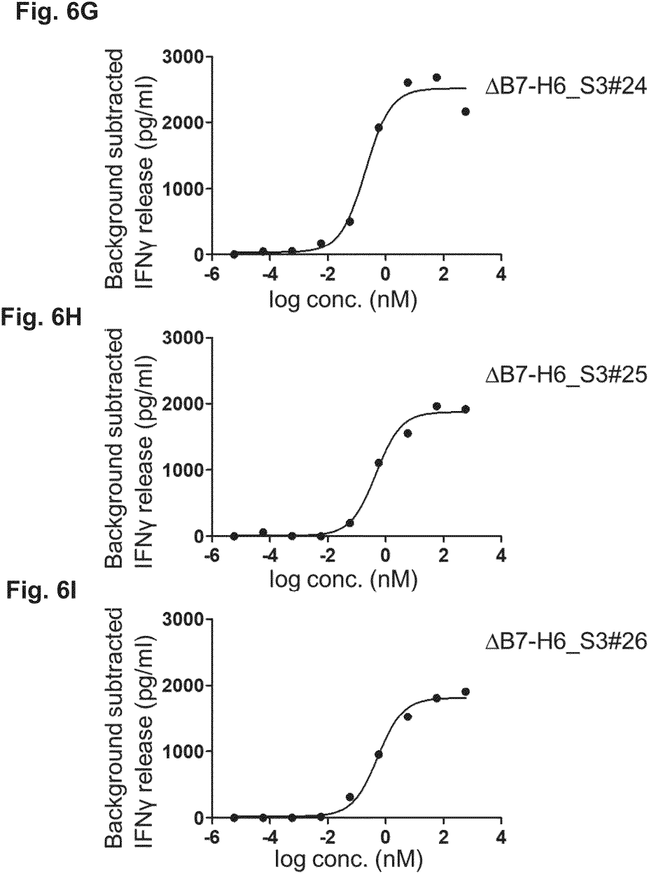

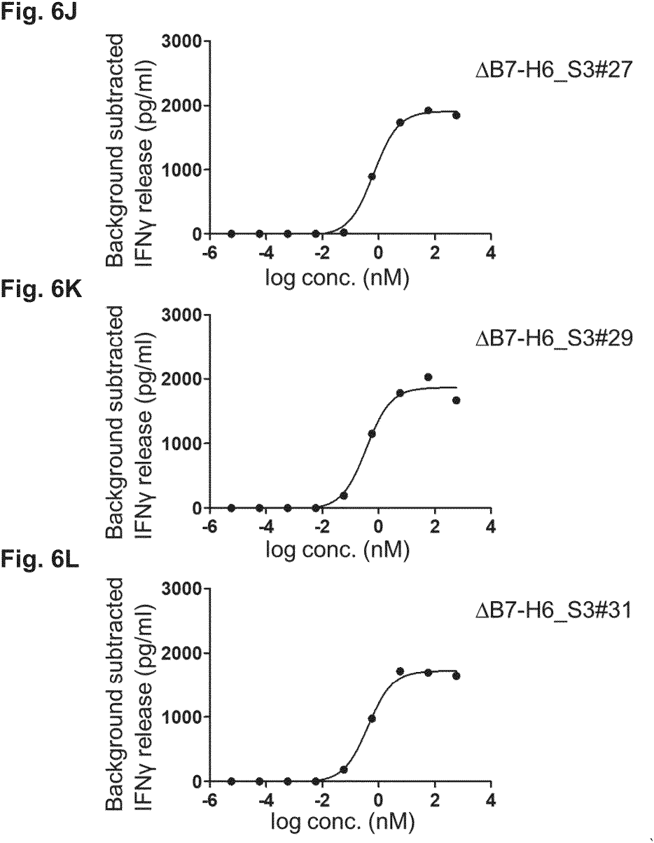

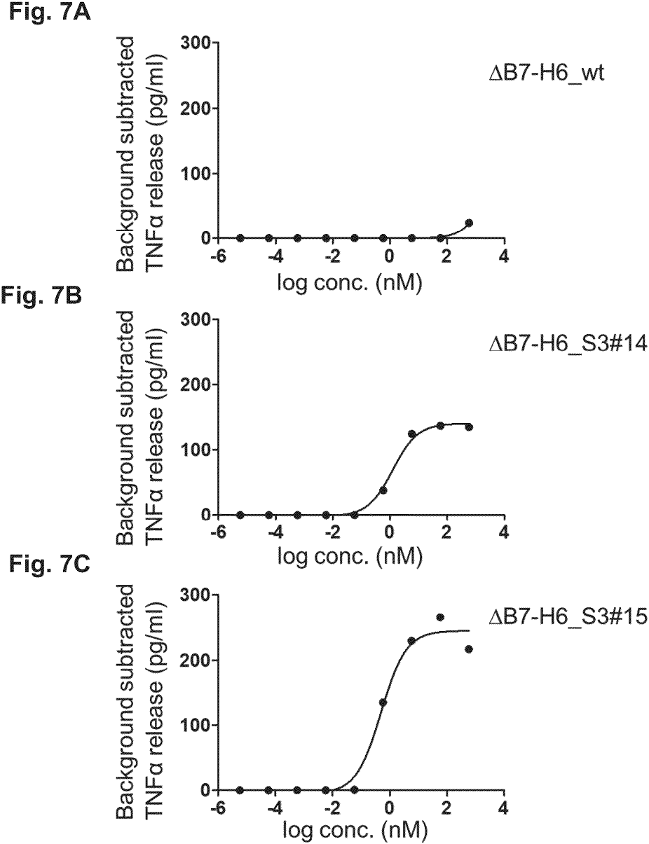

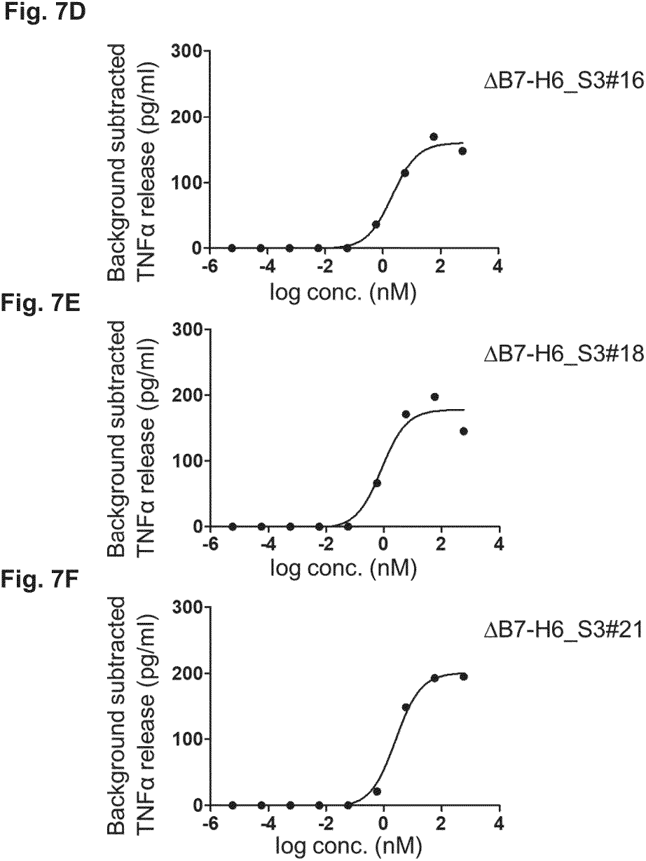

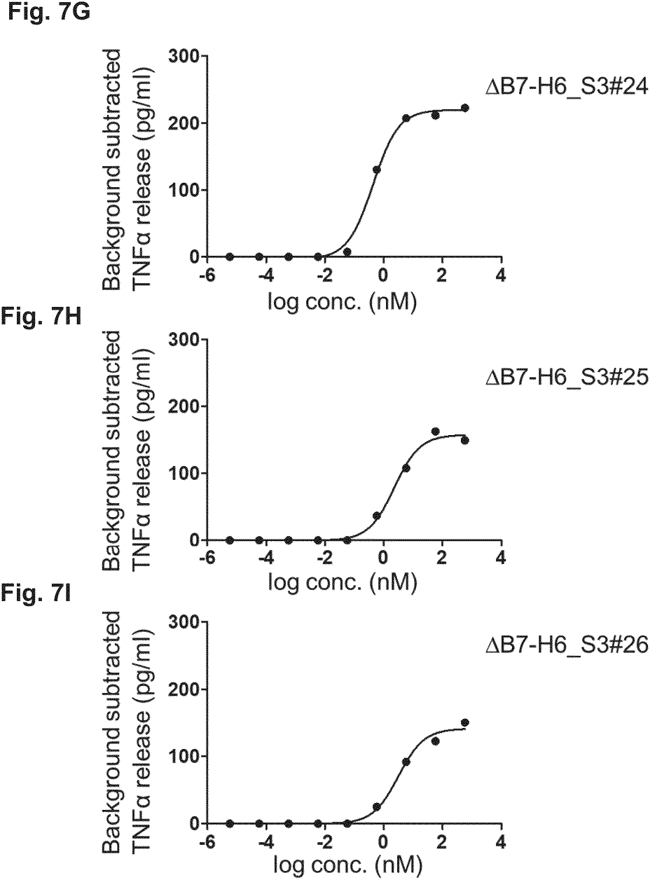

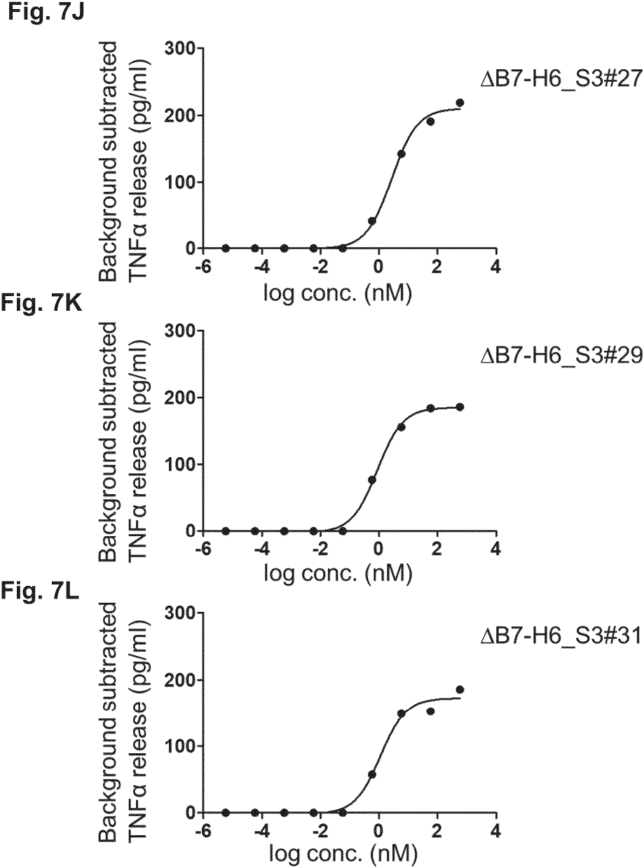

Representative dose response curves for NK cell mediated IFN-γ and TNF-α release obtained from one healthy donor with various affinity-maturated ΔB7-H6 immunoligands are shown in FIG. 6 and FIG. 7, respectively.

Table 7 below shows TNFα and IFNγ secretion data based on the preceding 24 h incubation and therefore activation of NK cells due to the tested bispecific antibody molecules. Assay supernatant was tested on TNFα und IFNγ secretion. It can be seen, that both cytokines are secreted by NK cells. Control values as “basal secretion” (co-cultivation of A431 and NK cells in absence of antibodies) of the cytokines were subtracted from the data, resulting in baseline corrected values. Moreover, neither TNFα nor IFNγ secretion was detected in the supernatant if NK cells were cultivated with EGFR-negative CHO cells, indicating that the increased cytokine release for A431 originates from a specific NK cell activation.

TABLE 7

Tumor necrosis factor alpha and interferon gamma secretion of activated natural killer cells

| Identifier | Max. TNFα release (pg/ml)^{∗∗∗} | TNFα release improvement compared to ΔB7-H6 wt | Max. IFNγ release (pg/ml)^{∗∗∗} | IFNγ release improvement compared to ΔB7-H6 wt |
|---|---|---|---|---|
| ΔB7-H6_wt | 12 | 1 | 510 | 1 |
| ΔB7-H6_S3#14 | 87 | 7.3 | 1284 | 2.5 |
| ΔB7-H6_S3#15 | 158 | 13.2 | 1926 | 3.8 |
| ΔB7-H6_S3#16 | 97 | 8.1 | 1507 | 3.0 |
| ΔB7-H6_S3#18 | 122 | 10.2 | 1607 | 3.2 |
| ΔB7-H6_S3#21 | 107 | 8.9 | 1472 | 2.9 |
| ΔB7-H6_S3#24 | 132 | 11.0 | 1763 | 3.5 |
| ΔB7-H6_S3#25 | 98 | 8.2 | 1427 | 2.8 |
| ΔB7-H6_S3#26 | 91 | 7.6 | 1341 | 2.6 |
| ΔB7-H6_S3#27 | 145 | 12.1 | 1448 | 2.8 |
| ΔB7-H6_S3#29 | 113 | 9.4 | 1337 | 2.6 |
| ΔB7-H6_S3#31 | 121 | 10.1 | 1339 | 2.6 |

^{∗∗∗}Values are calculated averages of two donors (n=2).

### Example 7

### ΔB7-H6 Affinity-Maturated NK Cell Engagers Elucidate Improved Killing by Concomitant Engagement of FcΔRIIIα

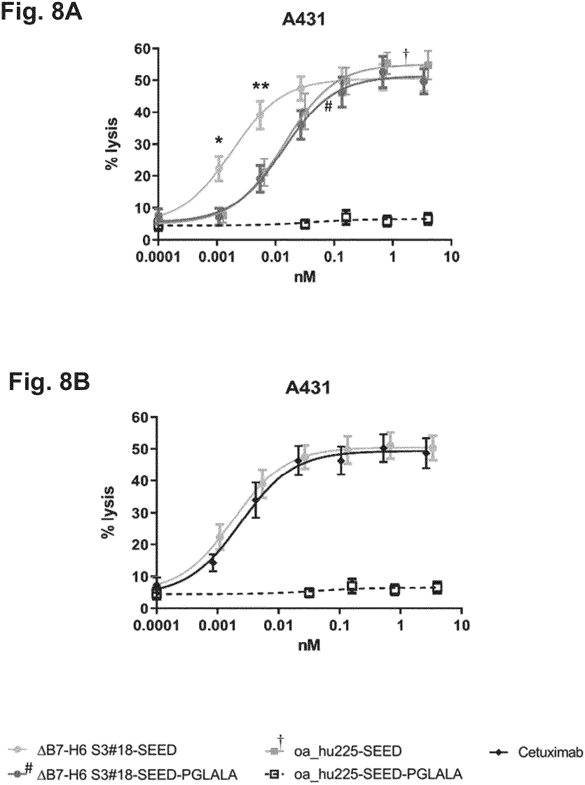

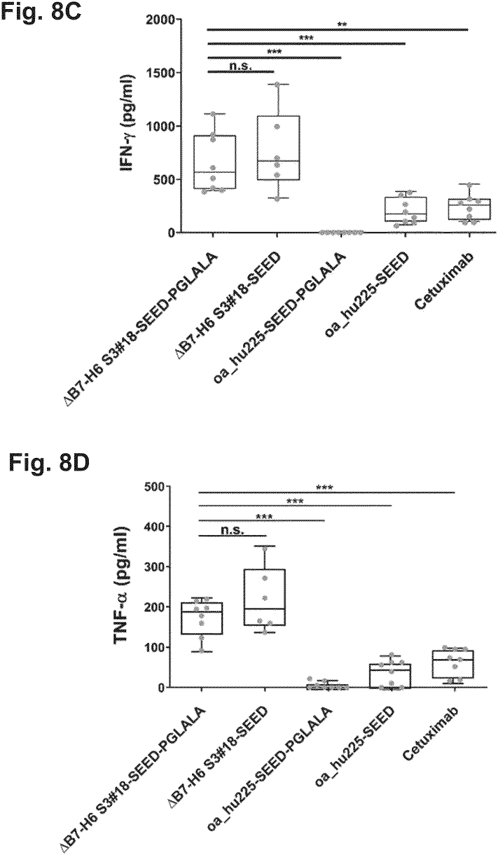

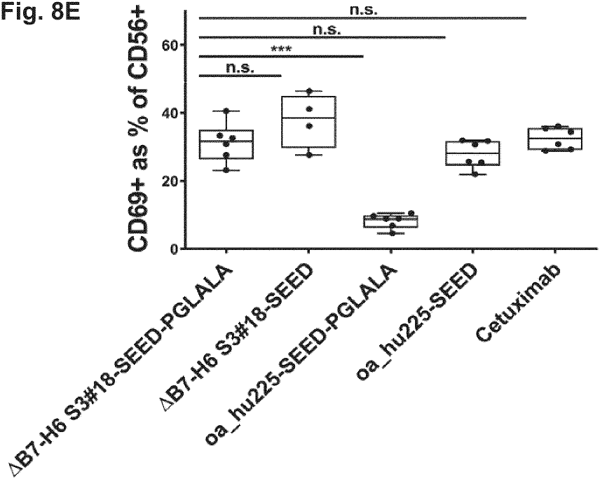

Finally, we tested the NK cell engager ΔB7H6 S#18, which has the highest binding affinity for NKp30 (KD = 9 nM), in the background of an Fc region capable of FcγRIIIa binding (SEEDbody lacking the PGLALA exchanges) to analyze whether tumor cell lysis can be further enhanced by concomitant NKp30 and FcγRIIIa activation of NK cells. The oa_hu225-SEED molecule harboring an Fc domain capable of FcR binding, but lacking a ΔB7-H6 domain, was as potent as the affinity-matured Fc-silenced ΔB7-H6 S3#18 SEED-PGLALA molecule, which solely activates NKp30 (FIG. 8A). Intriguingly, incorporation of a functional Fc in the NK cell engager S3#18 (i.e. ΔB7-H6 S3#18-SEED) significantly improved half-maximal killing by 9.2-fold (FIG. 8A). Furthermore, as shown in FIG. 8B, half-maximal killing achieved with ΔB7-H6 S3#18-SEED was in the range or even slightly exceeded the cytotoxic activity of the clinically applied antibody Cetuximab. These data give clear evidence that activating NK cells via NKp30 with an high affinity-engineered B7-H6-derived NK cell engager is as effective as activating NK cells via FcγRIIIa, and that the combination of NKp30 and FcγRIIIa activation of NK cells in one molecule can further potentiate the lysis of EGFR-expressing tumor cells.

Finally, we compared the capacity to trigger IFN-γ and TNF-α by the molecule engaging both NKp30 and FcγRIIIa with constructs engaging either NKp30 or FcyRIIIa and with the clinically approved antibody Cetuximab (FIGS. 8C, D). Interestingly, the capacity to trigger cytokine release was maintained in the molecule engaging both activating receptors compared to the molecule solely engaging NKp30, with a trend towards higher TNF-α release induced by the molecule with an effector competent IgG1 backbone. Of note, compared to the therapeutic antibody Cetuximab the release of IFN-γ and TNF-α was substantially elevated, irrespective of the utilized Fc. Moreover, activation of NK cells was comparable to Cetuximab (FIG. 8E) Ultimately, our data demonstrate that the herein engineered NK cell engagers are capable in triggering NK cell-mediated lysis similarly effective as the clinically approved antibody Cetuximab, but in addition triggers significant IFN-γ and TNF-α release that might be exploited to modulate anti-tumor immune responses.

### REFERENCES

P. André et al., “Anti-NKG2A mAb Is a Checkpoint Inhibitor that Promotes Anti-tumor Immunity by Unleashing Both T and NK Cells,” Cell (2018), vol. 175, no. 7, pp. 1731-1743.e13
D. M. Benson et al., “A Phase I Trial of the Anti-KIR Antibody IPH2101 and Lenalidomide in Patients with Relapsed/Refractory Multiple Myeloma,” Clinical Cancer Research (2015), vol. 21, no. 18, pp. 4055-4061
Y. T. Bryceson, M. E. March, D. F. Barber, H.-G. Ljunggren, and E. O. Long, “Cytolytic granule polarization and degranulation controlled by different receptors in resting NK cells,” The Journal of Experimental Medicine (2005), vol. 202, no. 7, pp. 1001-1012
M. C. Burger et al., “CAR-Engineered NK Cells for the Treatment of Glioblastoma: Turning Innate Effectors Into Precision Tools for Cancer Immunotherapy,” Frontiers in Immunology (2019), vol. 10, p. 2683
M. Carlsten and M. Järås, “Natural Killer Cells in Myeloid Malignancies: Immune Surveillance, NK Cell Dysfunction, and Pharmacological Opportunities to Bolster the Endogenous NK Cells,” Frontiers in Immunology (2019), vol. 10, p. 2357
L. Chiossone, P.-Y. Dumas, M. Vienne, and E. Vivier, “Natural killer cells and other innate lymphoid cells in cancer,” Nature Reviews Immunology (2018), vol. 18, no. 11, pp. 671-688
E. Coligan et al., “Current Protocols in Protein Science”, vol. 2 (1997), John Wiley & Sons, Inc.
K. Ellwanger et al., “Redirected optimized cell killing (ROCK®): A highly versatile multispecific fit-for-purpose antibody platform for engaging innate immunity,” mAbs (2019), vol. 11, no. 5, pp. 899-918
L. Gauthier et al., “Multifunctional Natural Killer Cell Engagers Targeting NKp46 Trigger Protective Tumor Immunity,” Cell (2019), vol. 177, no. 7, pp. 1701-1713.e16
Gonzalez-Rodriguez, Villa-Álvarez, Sordo-Bahamonde, Lorenzo-Herrero, and Gonzalez, “NK Cells in the Treatment of Hematological Malignancies,” Journal of Clinical Medicine (2019), vol. 8, no. 10, p. 1557
Green and Sambrook, “Molecular Cloning: A Laboratory Manual”, 4th ed. (2014), Cold Spring Harbor Laboratory Press (U.S.A.)
M.G. Joyce et al., “Crystal structure of human natural cytotoxicity receptor NKp30 and identification of its ligand binding site,” Proceedings of the National Academy of Sciences (2011), vol. 108, p. 6223-6228
C. Kellner et al., “Enhancing natural killer cell-mediated lysis of lymphoma cells by combining therapeutic antibodies with CD20-specific immunoligands engaging NKG2D or NKp30,” OncoImmunology (2016), vol. 5, no. 1, p. e1058459
J. Koch and M. Tesar, “Recombinant Antibodies to Arm Cytotoxic Lymphocytes in Cancer Immunotherapy,” Transfusion Medicine and Hemotherapy (2017), vol. 44, no. 5, pp. 337-350
H. E. Kohrt et al., “Anti-KIR antibody enhancement of anti-lymphoma activity of natural killer cells as monotherapy and in combination with anti-CD20 antibodies,” Blood (2014), vol. 123, no. 5, pp. 678-686
Y. Li et al., “Structure of the human activating natural cytotoxicity receptor NKp30 bound to its tumor cell ligand B7-H6,” The Journal of Experimental Medicine (2011), vol. 208 p. 703-714
S. Morgado et al., “NK Cell Recognition and Killing of Melanoma Cells Is Controlled by Multiple Activating Receptor-Ligand Interactions,” Journal of Innate Immunity (2011), vol. 3, no. 4, pp. 365-373
M. Peipp et al., “HER2-specific immunoligands engaging NKp30 or NKp80 trigger NK-cell-mediated lysis of tumor cells and enhance antibody-dependent cell-mediated cytotoxicity,” Oncotarget (2015), vol. 6, no. 31
R. Repp et al., “Combined Fc-protein- and Fc-glyco-engineering of scFv-Fc fusion proteins synergistically enhances CD16a binding but does not further enhance NK-cell mediated ADCC,” Journal of Immunological Methods (2011), vol. 373, p. 67-78
K. Rezvani, “Adoptive cell therapy using engineered natural killer cells,” Bone Marrow Transplantation (2019), vol. 54, no. S2, pp. 785-788
L. Roth et al., “Isolation of Antigen-Specific VHH Single-Domain Antibodies by Combining Animal Immunization with Yeast Surface Display,” Methods Mol. Biol. (2020), vol. 2070, p. 173-189
A. Rothe et al., “A phase 1 study of the bispecific anti-CD30/CD16A antibody construct AFM13 in patients with relapsed or refractory Hodgkin lymphoma,” Blood (2015), vol. 125, no. 26, pp. 4024-4031
T. Schlothauer et al., “Novel human IgG1 and IgG4 Fc-engineered antibodies with completely abolished immune effector functions,” Protein Engineering Design and Selection (2016), vol. 29, p. 457-466
U. J. E. Seidel, P. Schlegel, and P. Lang, “Natural Killer Cell Mediated Antibody-Dependent Cellular Cytotoxicity in Tumor Immunotherapy with Therapeutic Antibodies,” Frontiers in Immunology (2013), vol. 4
E. P. von Strandmann et al., “A novel bispecific protein (ULBP2-BB4) targeting the NKG2D receptor on natural killer (NK) cells and CD138 activates NK cells and has potent antitumor activity against human multiple myeloma in vitro and in vivo,” Blood (2006), vol. 107, no. 5, pp. 1955-1962
E. Vivier, E. Tomasello, M. Baratin, T. Walzer, and S. Ugolini, “Functions of natural killer cells,” Nat. Immunol. (2008), vol. 9, no. 5, pp. 503-510

1. A compound, comprising:
(a) a protein domain consisting of the amino acid sequence of SEQ ID NO: 3:
DLKVEMM4AGGQITPLNDMITIFCNTFYSQPLNITX₁MX₂WFA7KSLTFD
KEVKVFEFX₃X₄DHQEAFRPGAIVSPWRLKSGDASLRLPGIQLEEAGEYR
CEVX₅VX₆PX₇X₈AQGTVQLEVVASPAS (SEQ ID NO: 3),
wherein
X₁ is I, L, T, V, H, W, or Y;
X₂ is G;
X₃ is W or Y;
X₄ is G;
X₅ is V or I;
X₆ is T;
X₇ is Y, F, or L;
X₈ is K;
(b) a protein domain consisting of an amino acid sequence that is at least 75% identical to the amino acid sequence of the protein domain of (a); or
(c) a protein domain comprising a fragment of the protein domain of (a) or (b).

2. A compound, comprising;
(a) a protein domain consisting of the amino acid sequence of SEQ ID NO: 3:
DLKVEMMAGGTQITPLNDVNTIFCNIFYSQPLNITX₁ MX₂ ITWFWKSLTF
DKEVKVFEFX₃X₄ DHQEAFRPGAIVSPWRLKSGDASLRLPGIQLEEAGEY
RCEVX₅ VX₆ PX₇X₈ AQGTVQLEVVASPAS (SEQ ID NO: 3),
wherein
X₁ is H, E, S, T, I, or W;
X₂ is G;
X₃ is Y, W, or F;
X₄ is G or D;
X₅ is V, F, or I;
X₆ is T;
X₇ is Y or L;
X₈ is K;
(b) a protein domain consisting of an amino acid sequence that is at least 75% identical to the amino acid sequence of the protein domain of (a); or
(c) a protein domain comprising a fragment of the protein domain of (a) or (b).

3. A compound, comprising;
(a) a protein domain consisting of the amino acid sequence of SEQ ID NO: 3:
DTKIEIAMAGTQITPLNDITVTTFCNIFYSQPLNITX₁ MX₂ ITWFWKSLT
FDKEVKVFEFX₃X₄ DHQEAFRPGAIVSHRLKSGDASLRLPGIQIEEAGEY
RCEVX₅ VX₆ PX₇X₈ AQGTVQLEVVASPAS (SEQ ID NO: 3),
wherein
X₁ is E, S, H, T, L, or Q;
X₂ is G;
X₃ is W, Y, or F;
X₄ is G, A, D, or Q;
X₅ is V, I, or F;
X₆ is T;
X₇ is Y, L_{,} or V;
X₈ is K:
(b) a protein domain consisting of an amino acid sequence that is at least 75% identical to the amino acid sequence of the protein domain of (a); or
(c) a protein domain comprising a fragment of the protein domain of (a) or (b).

4. A compound, comprising;
(a) a protein domain consisting of the amino acid sequence of any one of the following sequences:

| SEQ ID NO | Amino acid sequence |
|---|---|
| SEQ ID NO: 4 | DLKVEMMAGGTQITPLNDNTIFCNIFYSQPLNITTMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 5 | DLKVEMMAGGTQITPLNDNVTIFCNIFYSQPLNITWMGITW FWKSLTFDKEVKVFEFWGDHQEAFRPGAIVSPWRLKSGDAS LRLPGIQLEEAGEYRCEVVVTPYKAQGTVQLEVVASPAS |
|---|---|
| SEQ ID NO: 6 | DLKVEMMAGGTQITPLNDNVTIFCNIFYSQPLNITHMGITW FWKSLTFDKEVKVEEFFGDHQEAFRPGAIVSPWRLESGDAS LRLPGIQLEEAGEYRCEVFVTPYKAQGTVQLEVVASPAS |
| SEQ ID NO: 7 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPLKAQGTVQLEVVASPAS |
| SEQ ID NO: 8 | DLKVEMMAGGTQITPLNDNVTIFCNIFYSQPLNITVMGITW FWKSLTPFDKEVKVFEFWGDHQEAFRPTIAIVSPWRUKSGDAS LRLPGIQLEEAGEYRCEVVVTPYKAQGTVQLEVVASPAS |
| SEQ ID NO: 9 | DLKVEMMAGGTQITPLNDNVTIFCNIFYSQPLNITHMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 10 | DLKVEMMAGGTQITPLNDNVTIFCNIFYSQPLNITHMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 11 | DLKVEMMAGGTQITPLNDNVTIFCNIFYSQPLNITHMGITW FWKSLTFDKEVKVEEEWGDHQEAFRPGAIVSPWRLESGDAS LRLPGIQLEEAGEYRCEVIVTPLKAQGTVQLEVVASPAS |
| SEQ ID NO: 12 | DLKVEMMAGGTQITPLNDNVTIFCNIFYSQPLNITHMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 13 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVFVTPYKAQGTVQLEVVASPAS |
| SEQ ID NO: 14 | DLKVEMMAGGTQITPLNDNVTIFCNIFYSQPLNITLMGITW FWKSLTFDKEVKVEEEWGDHQEAFRPGAIVSPWRLESGDAS LRLPGIQLEEAGEYRCEVIVTPFKAQGTVQLEVVASPAS |
| SEQ ID NO: 15 | DLKVEMMAGGTQITPLNDNVTIFCNIFYSQPLNITEMGITW FWKSLTFDKEVKVFEFFGDHQEAFRPGAIVSPWRLKSGDAS LRLPGIQLEEAGEYRCEVVVTPYKAQGTVQLEVVASPAS |
| SEQ ID NO: 17 | DLKVEMMAGGTQITPLNDNVTIFCNIFYSQPLNITTMGITW FWKSLTFOKEVKVFEFYGDHQEAFRPGAIVSPWRLKSGDAS LRLPGIQLEEAGEYRCEVVVTPYKAQGTVQLEVVASPAS |
| SEQ ID NO: 18 | DLKVEMMAGGTQITPLNDNVTIFCNIFYSQPLNITIMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 19 | DLKVEMMAGGTQITPLNDNVTIFCNIFYSQPLNITLMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 20 | DLKVEMMAGGTQITPLNDNVTIFCNIFYSQPLNITSMGITW FWKSLTFDKEVIVFEFYGDHQEAFRPGAIVSPWRLKSGDAS LRLPGIQLEEAGEYRCEVFVTPLKAQGTVQLEVVASPAS |
| SEQ ID NO: 21 | DLKVEMMAGGTQITPLNDNVTIFCNIFYSQPLNITYMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 22 | DLKVEMMAGGTQITPLNDNVTIFCNIFYSQPLNITQMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 23 | DLKVEMMAGGTQITPLNDNVTIFCNIFYSQPLNITHMGITW FWKSLTFDKEVKVEEEWGDHQEAFRPGAIVSPWRLESGDAS LRLPGIQLEEAGEYRCEVFVTPLKAQGTVQLEVVASPAS |
| SEQ ID NO: 24 | DLKVEMMAGGTQITPLNDNVTIFCNIFYSQPLNITLMGITW FWKSLTFDKEVKVFEFWGDHQEAFRPGAIVSPWRLKSGDAS LRLPGIQLEEAGEYRCEVVVTPYKAQGTVQLEVVASPAS |
| SEQ ID NO: 25 | DLKVEMMAGGTQITPLNDNVTIFCNIFYSQPLNITEMGITW FWKSLTFOKEVKVFEFWGDHOEAFRPGAIVSPWRLKSGDAS LRLPGIQLEEAGEYRCEVVVTPYKAQGTVQLEVVASPAS |
| SEQ ID NO: 26 | DLKVEMMAGGTQITPLNDNVTIFCNIFYSQPLNITEMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 27 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPYKAQGTVQLEVVASPAS |
| SEQ ID NO: 28 | DLKVEMMAGGTQITPLNDNVTIFCNIFYSQPLNITWMGITW FWKSLTFDKEVIVFEFYGDHQEAFRPGAIVSPWRLKSGDAS LRLPGIQLEEAGEYRCEVVVTPYKAQGTVQLEVVASPAS |
| SEQ ID NO: 29 | DLKVEMMAGGTQITPLNDNVTIFCNIFYSQPLNITTMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 30 | DLKVEMMAGGTQITPLNDNVTIFCNIFYSQPLNITTMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 31 | DLKVEMMAGGTQITPLNDNVTIFCNIFYSQPLNITHMGITW FWKSLTFDKEVKVEEEWGDHQEAFRPGAIVSPWRLESGDAS LRLPGIQLEEAGEYRCEVVVTPLKAQGTVQLEVVASPAS |
| SEQ ID NO: 32 | DLKVEMMAGGTQITPLNDNVTIFCNIFYSQPLNITVMGITW FWKSLTFDKEVKVFEFWGDHQEAFRPGAIVSPWRLKSGDAS LRLPGIQLEEAGEYRCEVIVTPYKAQGTVQLEVVASPAS |
| SEQ ID NO: 33 | DLKVEMMAGGTQITPLNDNVTIFCNIFYSQPLNITEMGITW FWKSLTFOKEVKVFEFWGDHOEAFRPGAIVSPWRLKSGDAS LRLPGIQLEEAGEYRCEVIVTPLKAQGTVQLEVVASPAS |
| SEQ ID NO: 34 | DLKVEMMAGGTQITPLNDNVTIFCNIFYSQPLNITIMGITW FWKSLTFDKEVKVFEFYGDHQEAFRPGAIVSPWRLKSGDAS LRLPGIQLEEAGEYRCEVVVTPLKAQGTVQLEVVASPAS |
| SEQ ID NO: 35 | DLKVEMMAGGTQITPLNDNVTIFCNIFYSQPLNITEMGITW FWKSLTFDKEVKVEFEFWGDHQEAFRPGATVSPWRLKSGDAS LRLPGIQLEEAGEYRCEVVVTPVKAQGTVQLEVVASPAS |
| SEQ ID NO: 36 | DLKVEMMAGGTQITPLNDNVTIFCNIFYSQPLNITSMGITW FWKSLTFDKEVIVFEFYGDHQEAFRPGAIVSPWRLKSGDAS LRLPGIQLEEAGEYRCEVVVTPYKAQGTVQLEVVASPAS |
| SEQ ID NO: 37 | DLKVEMMAGGTQITPLNDNVTIFCNIFYSQPLNITSMGITW FWKSLTFDKEVKVFEFWDDHQEAFRPGAIVSPWRLKSGDAS LRLPGIQLEEAGEYRCEVVVTPYKAQGTVQLEVVASPAS |
| SEQ ID NO: 38 | DLKVEMMAGGTQITPLNDNVTIFCNIFYSQPLNITTMGITW FWKSLTFDREVKVFEFWGDHQEAFRPGAIVSPWRLKSGDAS LRLPGIQLEEAGEYRCEVVVTPLKAQGTVQLEVVASPAS |
| SEQ ID NO: 39 | DLKVEMMAGGTQITPLNDNVTIFCNIFYSQPLNITHMGITW FWKSLTEFDKEVKVFEFYADHQEAIRPGATVSPWRLKSEGDAS LRLPGIQLEEAGEYRCEVVVTPYKAQGTVQLEVVASPAS |
| SEQ ID NO: 40 | DLKVEMMAGGTQITPLNDNVTIFCNIFYSQPLNITSMGITW LWKSLTFDKEVKVEFEFWGDHQEAFRPGAIVSPWRLKSGDAS LRLPGIQLEEAGEYRCEVFVTPYKAQGTVQLEVVASPAS |
| SEQ ID NO: 41 | DLKVEMMMAGGTQITPLNDNVTIFCNIFYFQPLNITEMGITW FWKSLTFOKEVKVFEFFGDHQEAFRPGAIVSPWRLKSGDAS LRLPGIQLEEAGEYRCEVIVTPLKAQGTVQLEVVASPAS |
| SEQ ID NO: 42 | DLKVEMMAGGTQITPLNDNVTIFCNIFYSQPLNITSMGITW FWKSLTFDKEVKVFEFYGDHQEAFRPGAIVSPWRLKSGDAS LRLPGIQLEEAGEYRCEVIVTPYKAQGTVQLEVVTSPAS |
| SEQ ID NO: 43 | DLKVEMMAGGTQITPLNDNVTIFCNIFYSQPLNITTMGITW FWKSLTFDKEVKVEFEFWGDHQEAFRPGATVSPWRLKSGDAS LRLPGIQLEEAGEYRCGVVVTPYKAQGTVQLEVVASPAS |
| SEQ ID NO: 44 | DLKVEMMAGGTQITPLNDNVTIFCNIFYSQPLNITSMGITW FWKSLTFDKEVIVFEFYGDHQEAFRPGAIVSPWRLKSGDAS LRLPGIQLEEAGEYRCEVVVTPLKAQGTAQLEVVASPAS |
| SEQ ID NO: 45 | DLKVEMMAGGTQITPLNDNVTIFCNIFYSQPLNITLMGITW FWKSLTFDKEVKVFEFWGDHQEAFRPGAIVSPWGLKSGDAS LRLPGIQLEEAGEYRCEVVVTPLKAQGTVQLEVVASPAS |
| | |
|---|---|
| SEQ ID NO: 46 | DLKVEMMAGGTQITPLNDNVTIFCNIFYSQPLNITTMGITW FWKSLTFDREVKVFEFWQDHQEAFRPGAIVSPWRLKSGDAS LRLPGIQLEEAGEYRCEVVVTPLKAQGTVQLEVVASPAS |
| SEQ ID NO: 47 | DLKVEMMAGGTQITPLNDNVTIFCNIFYSQPLNITHMGITW LWKSLTFDKEVIVFEFYGDHQEAFRPGAIVSPWRLKSGDAS LRLPGIQLEEAGEYRCEVVVTPLKAQGTVQLEVVASPAS |
| SEQ ID NO: 48 | DLKVEMMAGGTQITPLNDNVTIFCNIFYSQPLNITSMGITW FWKSLTFDKEVKVFEFWGDHQEARPGAIVSPWRLKSGDAS LRLPGIQLEEAGEYRCEVVVTPYKAQGTVQLEVVASPAS |
| SEQ ID NO: 49 | DLKVEMMAGGTQITPLNDNVTIFCNIFYSQPLNITTMGITW FWKSLTFOKEVKVFEFFGDHQEAFRPGAIVSPWRLKSGDAS LRLPGIQLEEAGEYRCEVVVTPYKAQGTVQLEVVASPAS |
| SEQ ID NO: 50 | DLKVEMMAGGTQITPLNDNVTIFCNIFYSQPLNITEMGITW FWKSLTFDKEVKVFEFYGDHQEAFXPGAIVSPWRLKSGDAS LXLPGIQLEEAGEYRCEVVVTPLKAQGTVQLEVVASPAS |

, or
(b) a protein domain consisting of an amino acid sequence that is identical to at least one sequence listed under (a) with the only difference that in up to 30 occasions an amino acid is individually added, replaced by another amino acid, or deleted in the amino acid sequence of the protein domain of (b) compared to said at least one sequence listed under (a), or
(c) a protein domain consisting of an amino acid sequence that is a fragment of the amino acid sequence of any one of the sequences listed in (a), or an amino acid sequence that is a fragment of an amino acid sequence that is identical to at least one sequence listed under (a) with the only difference that in up to 30 occasions an amino acid is individually added, replaced by another amino acid, or deleted in the amino acid sequence of the protein domain of (c) compared to said at least one sequence listed under (a).

5. The compound according to claim 1, wherein said compound further comprises a targeting moiety.

6. The compound according to claim 5, wherein said targeting moiety is a protein, a peptide, a peptide mimetic, a nucleic acid, an oligonucleotide, or a small molecule.

7. The compound according to claim 5, wherein said targeting moiety is an antibody or antigen-binding fragment of an antibody.

8. The compound according to claim 7, wherein said antibody is selected from the group consisting of an IgG antibody, an IgA antibody, an IgM antibody, and a hybrid thereof, and said antigen-binding fragment is selected from the group consisting of an IgG antibody, an IgA antibody, an IgM antibody, and a hybrid thereof.

9. The compound according to claim 5, wherein said targeting moiety is capable of specifically binding to a tumor-associated antigen.

10. The compound according to claim 9, wherein said tumor-associated antigen is selected from the group consisting of (epidermal growth factor receptor (EGFR), Human Epidermal Growth Factor Receptor 2 (HER2), (Programmed cell death 1 ligand 1(PD-L1), and CD20.

11. The compound according to claim 1, wherein said compound comprises an antibody Fc region competent in Fc receptor binding.

12. A pharmaceutical composition, comprising;
the compound according to claim 1, and
a pharmaceutically acceptable carrier, diluent, and/or excipient.

13. (canceled)

14. A method for preparing a compound with an increased affinity for NKp30 compared to a compound comprising a protein domain with the amino acid sequence of SEQ ID NO: 2:
DLKVEMMAGGTQITPLNDNVTIFCNIFYSQPLNITSMGITWEFWKSLTFD
KEVKVFEFFGDHQEAFRPGAIVSPWRLKSGDASLRLPGIQLEEAGEYRCE
VVVTPLKAQGTVQLEVVASPAS(SEQ ID NO: 2),
said method comprises:
preparing a compound comprising a variant of the protein domain with the amino acid sequence of SEQ ID NO: 2 in which at least one of the following amino acid replacements has been carried out compared to the sequence of SEQ ID NO: 2:
replacement of X₁ by I, L, T, V, H, W, Y, E, or Q;
replacement of X₃ by W or Y;
replacement of X₄ by D, A, or Q;
replacement of X₅ by I or F;
replacement of X₇ by Y, F, or V.
wherein X₁, X₃, X₄, X₅, and X₇ refer to the following positions within the sequence of SEQ ID NO: 2:
DLKVEMMAGGTQITPLNDIPITIFCNIFYSQPLNITX₁ MGITWFWKSTTF
DKEVKVFEFX₃X₄ DHQEAFRPGAIVSPWRLKSGDASLRTPGTQLEEAGEY
RCEVX₅ VTPX₇ KAQGTVQLEVVASPAS.

15. The method according to claim 14, wherein the method comprises preparing the compound comprising the variant of the protein domain with the amino acid sequence of SEQ ID NO: 2 in which the amino acids at the positions X₁ to X₈ of SEQ ID NO: 2 have been replaced compared to the sequence of SEQ ID NO: 2 by the same combination of amino acids as in one of SEQ ID NO: 4 to 15 or 17 to 50:

| | | | | | | | | |
|---|---|---|---|---|---|---|---|---|
| | X₁ | X₂ | X₃ | X₄ | X₅ | X₆ | X₇ | X₈ |
| SEQ ID NO: 4 | T | G | W | G | V | T | Y | K |
| SEQ ID NO: 5 | W | G | W | G | V | T | Y | K |
| SEQ ID NO: 6 | H | G | F | G | F | T | Y | K |
| SEQ ID NO: 7 | I | G | W | G | V | T | L | K |
| SEQ ID NO: 8 | V | G | W | G | V | T | Y | K |
| SEQ ID NO: 9 | H | G | W | G | V | T | Y | K |
| SEQ ID NO: 10 | H | G | Y | G | V | T | Y | K |
| SEQ ID NO: 11 | H | G | W | G | I | T | L | K |
| SEQ ID NO: 12 | H | G | F | G | V | T | Y | K |
| SEQ ID NO: 13 | S | G | Y | G | F | T | Y | K |
| SEQ ID NO: 14 | L | G | W | G | I | T | F | K |
| SEQ ID NO: 15 | E | G | F | G | V | T | Y | K |
| SEQ ID NO: 17 | T | G | Y | G | V | T | Y | K |
| SEQ ID NO: 18 | I | G | W | G | V | T | Y | K |
| SEQ ID NO: 19 | L | G | Y | G | V | T | Y | K |
| SEQ ID NO: 20 | S | G | Y | G | F | T | L | K |
| SEQ ID NO: 21 | Y | G | W | G | V | T | Y | K |
| SEQ ID NO: 22 | Q | G | W | G | V | T | L | K |
| SEQ ID NO: 23 | H | G | W | G | F | T | Y | K |
| SEQ ID NO: 24 | L | G | W | G | V | T | Y | K |
| SEQ ID NO: 25 | E | G | W | G | V | T | Y | K |
| SEQ ID NO: 26 | E | G | Y | G | V | T | Y | K |
| SEQ ID NO: 27 | I | G | Y | G | V | T | Y | K |
| SEQ ID NO: 28 | W | G | Y | G | V | T | Y | K |
| SEQ ID NO: 29 | T | G | W | D | V | T | Y | K |
| SEQ ID NO: 30 | T | G | W | G | I | T | Y | K |
| SEQ ID NO: 31 | H | G | W | G | T | T | L | K |
| SEQ ID NO: 32 | V | G | W | G | T | T | Y | K |
| SEQ ID NO: 33 | E | G | W | G | T | T | L | K |
| SEQ ID NO: 34 | I | G | Y | G | V | T | L | K |
| SEQ ID NO: 35 | E | G | W | G | V | T | V | K |
| SEQ ID NO: 36 | S | G | Y | G | V | T | Y | K |
| SEQ ID NO: 37 | S | G | W | D | V | T | Y | K |
| SEQ ID NO: 38 | T | G | W | G | V | T | L | K |
| SEQ ID NO: 39 | H | G | Y | A | V | T | Y | K |
| SEQ ID NO: 40 | S | G | W | G | F | T | Y | K |
| SEQ ID NO: 41 | E | G | F | G | I | T | L | K |
| SEQ ID NO: 42 | S | G | Y | G | I | T | Y | K |
| SEQ ID NO: 43 | T | G | W | G | V | T | V | K |
| SEQ ID NO: 44 | S | G | Y | G | V | T | L | K |
| SEQ ID NO: 45 | L | G | W | G | V | T | L | K |
| SEQ ID NO: 46 | T | G | W | Q | V | T | L | K |
| SEQ ID NO: 47 | H | G | Y | G | V | T | L | K |
| SEQ ID NO: 48 | S | G | W | G | V | T | Y | K |
| SEQ ID NO: 49 | T | G | F | G | V | T | Y | K |
| SEQ ID NO: 50 | E | G | Y | G | V | T | L | K |

, wherein Xi to X
₈ refers to the following positions within the sequence of SEQ ID NO: 2:
DLKVEMMAGGTQITPLNDNVTI FCNIFYSQPLNITX₁ MX₂ ITWFWKSLT
FDKEVKVFEFX₃X₄ DHQEAFRPGAIVSPWRLKSGDASLRLPGIQLEEAGE
YRCEVX₅ VX₆ PX₇X₈ ACGTVQLEVVASPAS.
